(19) United States
(12) Patent Application Publication
(10) Pub. No.: US 2022/0323012 A1
(43) Pub. Date: Oct. 13, 2022

(54) SENSOR INTEGRATION IN CARDIAC IMPLANT DEVICES

(71) Applicant: Edwards Lifesciences Corporation, Irvine, CA (US)

(72) Inventors: Scott Louis Pool, Laguna Hills, CA (US); Steven McConnell, Anaheim, CA (US); Behnood Miri, Irvine, CA (US); Daniel James Murray, Orange, CA (US); Jose Islas, Corona, CA (US); Michael G Valdez, Riverside, CA (US)

(21) Appl. No.: 17/728,892

(22) Filed: Apr. 25, 2022

Related U.S. Application Data
(63) Continuation of application No. PCT/US2020/056746, filed on Oct. 22, 2020.
(60) Provisional application No. 62/926,829, filed on Oct. 28, 2019.

(51) Int. Cl. A61B 5/00; A61B 5/0215

(52) U.S. Cl. CPC A61B 5/6869; A61B 5/02158; A61B 5/6884

(57) ABSTRACT

A sensor-retention structure includes a sensor-support strut and a means for securing a sensor device to the sensor-support strut. A sensor-support arm can be configured to have disposed thereon an at least partially cylindrical sensor device and be associated with one or more sensor-retention fingers projecting from the sensor-support arm and/or a cage structure configured to be secured to the sensor device.

### RELATED APPLICATIONS

This application is a continuation application of PCT International Patent Application Serial No. PCT/US2020/056746, filed Oct. 22, 2020 and entitled SENSOR INTEGRATION IN CARDIAC IMPLANT DEVICES, which claims priority based on U.S. Provisional Patent Application No. 62/926,829, filed Oct. 28, 2019 and entitled SENSOR INTEGRATION IN CARDIAC IMPLANT DEVICES, the complete disclosures of both of which are hereby incorporated by reference in their entireties.

### BACKGROUND

### Field

The present disclosure generally relates to the field of a medical implant devices.

### Description of Related Art

Various medical procedures involve the implantation of a medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with the left atrium using one or more sensor implant devices implanted in or to one or more pulmonary veins and/or associated anatomy/tissue.

In some implementations, the present disclosure relates to a sensor-retention structure comprising a sensor-support arm configured to have disposed thereon an at least partially cylindrical sensor device and one or more sensor-retention fingers projecting from the sensor-support arm and configured to be secured to the sensor device.

The one or more sensor-retention fingers can be configured to be wrapped at least partially around a sensor device disposed on the sensor-support arm. In some embodiments, at least one of the one or more sensor-retention fingers comprises a strap form and at least one of the one or more sensor-retention fingers comprises a buckle form that extends from an opposing side of the sensor-support arm such that the strap form can be inserted through a portion of the buckle form. In some embodiments, at least one of the one or more sensor-retention fingers has an aperture therein dimensioned to allow for disposition of adhesive therein to secure the at least one of the one or more sensor-retention fingers to the sensor device. In some embodiments, the one or more sensor-retention fingers are positioned in one or sets of aligned opposing fingers.

The one or more sensor-retention fingers project distally from the sensor-support arm in some embodiments. For example, the one or more sensor-retention fingers include respective distal cross pieces. In some embodiments, at least two of the one or more sensor-retention fingers are configured to be locked together at distal ends thereof.

The sensor-retention structure can further comprise a distal stopper associated with a distal end portion of the sensor-support arm. In some embodiments, the sensor-retention structure further comprises an encasement form configured to be disposed at least partially over the sensor device when the sensor device is disposed on the sensor-support arm. For example, the encasement form can include one or more cutouts configured to fit at least one of the one or more sensor-retention fingers. In some embodiments, the one or more sensor-retention fingers are part of a removable partial ring form. In some embodiments, the one or more sensor-retention fingers have associated therewith respective tabs that are configured to be projected radially inward.

In some implementations, the present disclosure relates to a sensor-retention structure comprising a sensor-support arm configured to have disposed thereon an at least partially cylindrical sensor device and a cage structure associated with the sensor-support arm. The cage structure is configured to wrap at least partially around a circumferential surface of the sensor device.

In some embodiments, the sensor-support arm is attached to a proximal portion of a shunt arm structure and the sensor-support arm is configured to be bent away from the shunt arm structure to thereby project at least partially radially away from a longitudinal axis of the shunt arm structure. The cage structure can include one or more distal stopper tabs. In some embodiments, the cage structure comprises a plurality of longitudinal struts. For example, the cage structure can comprise a plurality of lateral struts connected between two or more of the plurality of longitudinal struts. The sensor-retention structure can further comprise a sleeve disposed around at least part of the cage structure, wherein the cage structure is in an at least partially wrapped sensor-retention configuration. In some embodiments, the sensor-retention structure further comprises a plurality of suture-attachment tabs associated with a distal end of the cage structure.

In some implementations, the present disclosure relates to a sensor-retention structure comprising a sensor-support strut and a means for securing a sensor device to the sensor-support strut. The means for securing the sensor device to the support strut can have any form, shape, composition, and/or configuration of or relating to any aspect of any of the embodiments illustrated and/or disclosed herein.

The means for securing the sensor device to the sensor support strut can comprise one or more strap features associated with the sensor-support strut. In some embodiments, the means for securing the sensor device to the sensor support strut comprises a cloth wrapped around at least a portion of the sensor device and the sensor-support strut. In some embodiments, the means for securing the sensor device to the sensor support strut comprises a polymer film disposed around at least a portion of the sensor device and the sensor-support strut. In some embodiments, the means for securing the sensor device to the sensor support strut comprises a sensor mold associated with the sensor-support strut, wherein the sensor mold is configured to have the sensor device inserted therein.

The means for securing a sensor device to the sensor-support strut can include a proximal sensor-attachment structure that projects at least partially orthogonally from the sensor-support strut. For example, the proximal sensor-attachment structure can include an aperture therein dimensioned to allow for disposition of adhesive therein to secure the proximal sensor-attachment structure to the sensor device. In some embodiments, the sensor-attachment structure comprises a suction cup associated with a distal side thereof. In some embodiments, the proximal sensor-attachment structure comprises a distally-angled arm having a hook feature associated with a distal end thereof.

In some embodiments, the means for securing the sensor device to the sensor support strut comprises a housing-attachment flange and a sensor housing configured to be attached at a proximal end thereof to the housing-attachment flange and to house the sensor device. The means for securing the sensor device to the sensor support strut can comprise one or more trapdoor flaps. In some embodiments, the means for securing the sensor device to the sensor support strut comprises a sheet configured to be wrapped around at least a portion of the sensor device.

In some embodiments, the means for securing the sensor device to the sensor support strut comprises one or more hoop forms configured to be bent away from the sensor-support strut and have sensor device disposed at least partially through openings of the one or more hoop forms. In some embodiments, the means for securing the sensor device to the sensor support strut comprises a locking retention arm including a plurality of prongs and a sensor housing including a plurality of channels associated with a proximal end thereof and configured to receive one or more of the plurality of prongs and a distal end portion of the sensor-support strut.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example representation of a human heart in accordance with one or more embodiments.

FIG. 2 illustrates example pressure waveforms associated with various chambers and vessels of the heart according to one or more embodiments.

FIG. 3 is a block diagram representing an implant device in accordance with one or more embodiments.

FIG. 4 is a block diagram representing a system for monitoring one or more physiological parameters associated with a patient according to one or more embodiments.

FIG. 5 illustrates an example shunt structure in accordance with one or more embodiments.

FIG. 6 illustrates a sensor implant device incorporated with and/or attached/secured to a medical device structure in accordance with one or more embodiments.

FIG. 7 shows a sensor implant device implanted in an atrial septum in accordance with one or more embodiments.

FIG. 8 shows a sensor implant device implanted in a tissue wall between the coronary sinus and the left atrium in accordance with one or more embodiments.

FIG. 9 shows a perspective view of a shunt device in a catheter-delivery (e.g., at least partially collapsed) configuration in accordance with one or more embodiments.

FIG. 10 illustrates a sensor implant device having sensor-support strut or arm in accordance with one or more embodiments.

FIGS. 11A and 11B show perspective views of a medical implant device including a sensor-support strut/arm and a fabric and/or polymer cloth wrap configured to at least partially secure a sensor device to the sensor-support strut/arm in accordance with one or more embodiments.

FIGS. 12A and 12B show perspective views of a medical implant device including a sensor-support strut/arm and a membrane wrap configured to at least partially secure a sensor device to the sensor-support strut/arm in accordance with one or more embodiments

FIGS. 13A and 13B illustrate perspective views of a sensor implant device having an integrated sensor that is secured to a sock feature in accordance with one or more embodiments.

FIGS. 14A and 14B show perspective views of a medical implant device including a sensor-support strut/arm and a membrane wrap configured to at least partially secure a sensor device to the sensor-support strut/arm in accordance with one or more embodiments.

FIGS. 15A and 15B show perspective views of a medical implant devices including respective sensor-support strut/arms and buckle and/or strap member(s) associated therewith in accordance with one or more embodiments.

FIGS. 16A and 16B show perspective views of a medical implant device including a sensor-support strut/arm and one or more buckle and/or strap members associated therewith and/or one or more axial retention features in accordance with one or more embodiments.

FIG. 16C shows a side view of the medical implant device of FIGS. 16A and 16B in accordance with one or more embodiments.

FIGS. 17A-17C show perspective and end views, respectively, of at least a portion of a medical implant device including a sensor-support strut/arm and one or more sensor-retention fingers in accordance with one or more embodiments.

FIGS. 18A-18C show perspective views of a sensor-support strut/arm and a sensor-retention over-mold support form in accordance with one or more embodiments.

FIGS. 19A-19E show perspective, side, and end views, respectively, of a sensor-support strut/arm associated with a plurality of locking sensor retention fingers in accordance with one or more embodiments.

FIGS. 20A-20E show exploded, side, cross-sectional, and end views, respectively, of an arm structure associated with a housing-attachment flange in accordance with one or more embodiments.

FIGS. 21A and 21B show exploded and perspective views, respectively, an arm structure associated with one or more sets of sensor-retention and/or housing-connection fingers in accordance with one or more embodiments.

FIGS. 22A and 22B show perspective views of a medical implant device including a sensor-support strut/arm associated with a plurality of mechanically-locking sensor retention fingers in accordance with one or more embodiments.

FIGS. 23A and 23B show perspective views of a sensor-support strut/arm associated with a plurality of sets of sensor retention fingers in accordance with one or more embodiments.

FIGS. 24A and 24B show perspective views of a sensor-support structure including a trapdoor sensor-retention feature in accordance with one or more embodiments.

FIGS. 25A-25C illustrate perspective views of certain portions of a sensor-support arm associated with one or more tension-fit rings in accordance with one or more embodiments.

FIGS. 26A-26E illustrate perspective views of a sensor-support arm including one or more clamps features in accordance with one or more embodiments.

FIGS. 27A and 27B illustrate perspective views of a sensor-support arm structure including opposing circumferentially-wrapping fingers in accordance with one or more embodiments.

FIGS. 28A and 28B illustrate perspective views of a sensor-support structure including one or more prongs in accordance with one or more embodiments.

FIGS. 29A-29D illustrate perspective views of a sensor-support structure/arm including a proximal stopper feature in accordance with one or more embodiments.

FIGS. 30A-30D illustrate perspective and side views, respectively, of a sensor-retention structure including a sensor-support structure/arm and a suction-enabled proximal stopper feature in accordance with one or more embodiments.

FIGS. 31A-31D illustrate perspective and side views, respectively, of a sensor-retention structure that includes a proximally-positioned and distally-angled stopper and/or retention arm/structure in accordance with one or more embodiments.

FIGS. 32A and 32B illustrate perspective views of a sensor-retention structure including a sheet configured to be wrapped at least partially around a sensor device in accordance with one or more embodiments.

FIGS. 33A and 33B illustrate perspective views of a sensor-retention structure including a sensor-support arm structure and one or more sensor-retention rings in accordance with one or more embodiments.

FIGS. 34A-34D illustrate top, bottom, and side views, respectively, of a sensor-retention structure in accordance with one or more embodiments.

FIGS. 35A and 35B illustrate perspective views of a sensor-retention structure in accordance with one or more embodiments.

FIGS. 36A and 36B illustrate side and axial views, respectively, of a medical implant device comprising certain sensor-retention features in accordance with one or more embodiments.

FIGS. 37A and 37B illustrate perspective views of a sensor-retention structure including a sensor-retention cage structure in accordance with one or more embodiments.

FIGS. 38A and 38B illustrate perspective views of a sensor-retention structure configured to be bent away from an arm of a medical implant device in accordance with one or more embodiments.

FIG. 38C shows the sensor-retention structure of FIGS. 38A and 38B in a flat configuration in accordance with one or more embodiments.

FIG. 39 illustrates a side view of a medical implant device including an axial sensor-support arm configured to support a sensor device in accordance with one or more embodiments.

FIG. 40 shows a side view of an embodiment of a shunt-type medical implant device having a sensor device secured at least in part thereto in accordance with one or more embodiments.

FIGS. 41A-41C illustrate an embodiment of a shunt-type medical implant device having a sensor device secured at least in part thereto in accordance with one or more embodiments.

FIGS. 42A and 42B illustrate side and axial views, respectively, of an embodiment of a shunt-type medical implant device including certain sensor retention features in accordance with one or more embodiments.

FIG. 43 illustrates a perspective view of a sensor-retention structure including a sensor-retention trough in accordance with one or more embodiments.

FIG. 44 illustrates a perspective view of a sensor-retention structure including netting or the like configured to secure a sensor device to a sensor-support structure in accordance with one or more embodiments.

FIG. 45 illustrates a perspective view of a magnetic sensor-retention structure in accordance with one or more embodiments.

FIGS. 46A and 46B illustrate side perspective views of a sensor-retention structure in relatively shortened and lengthened configurations, respectively, in accordance with one or more embodiments.

FIGS. 47-49 illustrate separate embodiments of sensor structures including respective sensor-retention features in accordance with one or more aspects of the present disclosure.

FIGS. 50A-50E illustrate perspective and end views, respectively, of a sensor-retention structure including one or more sensor-retention fingers in accordance with one or more embodiments.

FIGS. 51A-51E illustrate a sensor-retention structure including one or more runner-type sensor-support struts in accordance with one or more embodiments.

FIGS. 52A-52C illustrate a sensor-retention structure including one or more runner-type sensor-support struts in accordance with one or more embodiments.

FIGS. 53A-53E illustrate perspective and side views, respectively, of embodiments of sensor-retention structures in accordance with one or more embodiments.

FIGS. 54A-54C illustrate perspective views of a sensor-retention structure including sensor-retention arms with end crosspieces in accordance with one or more embodiments.

FIG. 55 illustrates a perspective view of the sensor-retention structure of FIGS. 54A-54C with a sleeve disposed thereon in accordance with one or more embodiments.

FIGS. 56A-56D illustrate perspective views of a sensor-retention structure including axially-extending arms with end crosspieces in accordance with one or more embodiments.

FIG. 57 illustrates a perspective view of the sensor-retention structure of FIGS. 56A-56D with a sleeve disposed thereon in accordance with one or more embodiments.

FIGS. 58A-58D illustrate perspective views of a sensor-retention structure including a cage configured to hold a sensor device in accordance with one or more embodiments.

FIG. 59 illustrates a perspective view of the sensor-retention structure of FIGS. 58A-58D with a sleeve disposed thereon in accordance with one or more embodiments.

FIGS. 60A-60D illustrate perspective views of a sensor-retention structure including a cage structure in accordance with one or more embodiments.

FIG. 61 illustrates a perspective view of the sensor-retention structure 600 of FIGS. 60A-60D with a sleeve disposed thereon in accordance with one or more embodiments.

FIGS. 62A-62D illustrate perspective views of a sensor-retention structure including a cage structure in accordance with one or more embodiments.

FIG. 63 illustrates a perspective view of the sensor-retention structure 620 of FIGS. 62A-62D with a sleeve disposed thereon in accordance with one or more embodiments.

FIGS. 64A-64D illustrate perspective views of a sensor-retention structure including a cage structure in accordance with one or more embodiments.

FIG. 65 illustrates a perspective view of the sensor-retention structure of FIGS. 64A-64D with a sleeve disposed thereon in accordance with one or more embodiments.

FIGS. 66A and 66B illustrate perspective views of a sensor-retention structure including two support struts an arm of a medical implant device in accordance with one or more embodiments.

FIG. 67A-67C illustrate perspective views of the sensor-retention structure of FIGS. 66A-66B with a sleeve disposed thereon in accordance with one or more embodiments.

FIGS. 68A-68C illustrate perspective views of a sensor-retention structure including a cage structure in accordance with one or more embodiments.

FIGS. 69A-69D illustrate perspective views of a sensor-retention structure including a cage structure configured to hold a sensor device in accordance with one or more embodiments.

FIGS. 70A-70D illustrate perspective views of a sensor-retention structure including a cage structure and a distal stopper ring in accordance with one or more embodiments.

FIGS. 71A and 71B illustrate perspective views of a sensor-retention structure including a cage structure configured to hold a sensor device and one or more distal and/or proximal stopper features in accordance with one or more embodiments.

FIG. 72 illustrates a perspective view of a sensor-support strut and associated sensor covering in accordance with one or more embodiments.

FIGS. 73-75 illustrate bottom and perspective views, respectively, of embodiments of sensor-retention structures including a sensor-support strut and an encasement in accordance with one or more embodiments.

FIGS. 76A-76D illustrate exploded and perspective views, respectively, of a sensor-retention structure including a sensor-support arm and an encasement configured to fit or engage with the sensor-support arm in accordance with one or more embodiments.

FIG. 77 illustrates a perspective view of the sensor-retention structure of FIGS. 76A-76D with a sleeve disposed thereon in accordance with one or more embodiments.

FIGS. 78A-78D illustrate exploded and perspective views, respectively, of a sensor-retention structure including a sensor-support arm, an encasement configured to fit or engage with the sensor-support arm, and one or more sets of opposing fingers and/or one or more axially-offset retention fingers in accordance with one or more embodiments.

FIG. 79 illustrates a perspective view of the sensor-retention structure of FIGS. 78A-78D with a sleeve disposed thereon in accordance with one or more embodiments.

FIGS. 80A-80D illustrate exploded and perspective views, respectively, of a sensor-retention structure including a sensor-support arm, an encasement configured to fit or engage with the sensor-support arm, and one or more sets of opposing fingers with end crosspieces and/or one or more axially-offset retention fingers in accordance with one or more embodiments.

FIG. 81 illustrates a perspective view of the sensor-retention structure of FIGS. 80A-80D with a sleeve disposed thereon in accordance with one or more embodiments.

FIG. 82 illustrates a perspective view of a locking retention arm in accordance with one or more embodiments.

FIGS. 83A-83C illustrate views of a sensor-retention structure that is configured to be secured in some manner to a retention arm structure in accordance with one or more embodiments.

FIGS. 84A-84C show perspective views of at least a portion of the locking retention arm of FIG. 82 in accordance with one or more embodiments.

FIGS. 85A-85C represent stages of a process for inserting the locking retention arm of FIG. 82 into a proximal end portion of the retention structure of FIGS. 83A-83C in accordance with one or more embodiments.

FIGS. 86A-86D illustrate perspective views of a sensor-retention structure configured to hold a sensor device in accordance with one or more embodiments.

FIG. 87 is an end view of a sensor-retention structure including a distal stopper bar feature in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for telemetric monitoring of one or more physiological parameters of a patient (e.g., blood pressure) in connection with cardiac shunts and/or other medical implant devices and/or procedures. Such pressure monitoring may be performed using cardiac implant devices having integrated pressure sensors and/or associated components. For example, in some implementations, the present disclosure relates to cardiac shunts and/or other cardiac implant devices that incorporate or are associated with pressure sensors or other sensor devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain embodiments are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The contraction of the various heart muscles may be prompted by signals generated by the electrical system of the heart, which is discussed in detail below. Certain embodiments disclosed herein relate to conditions of the heart, such as atrial fibrillation and/or complications or solutions associated therewith. However, embodiments of the present disclosure relate more generally to any health complications relating to fluid overload in a patient, such as may result post-operatively after any surgery involving fluid supplementation. That is, detection of atrial stretching as described herein may be implemented to detect/determine a fluid-overload condition, which may direct treatment or compensatory action relating to atrial fibrillation and/or any other condition caused at least in part by fluid overloading.

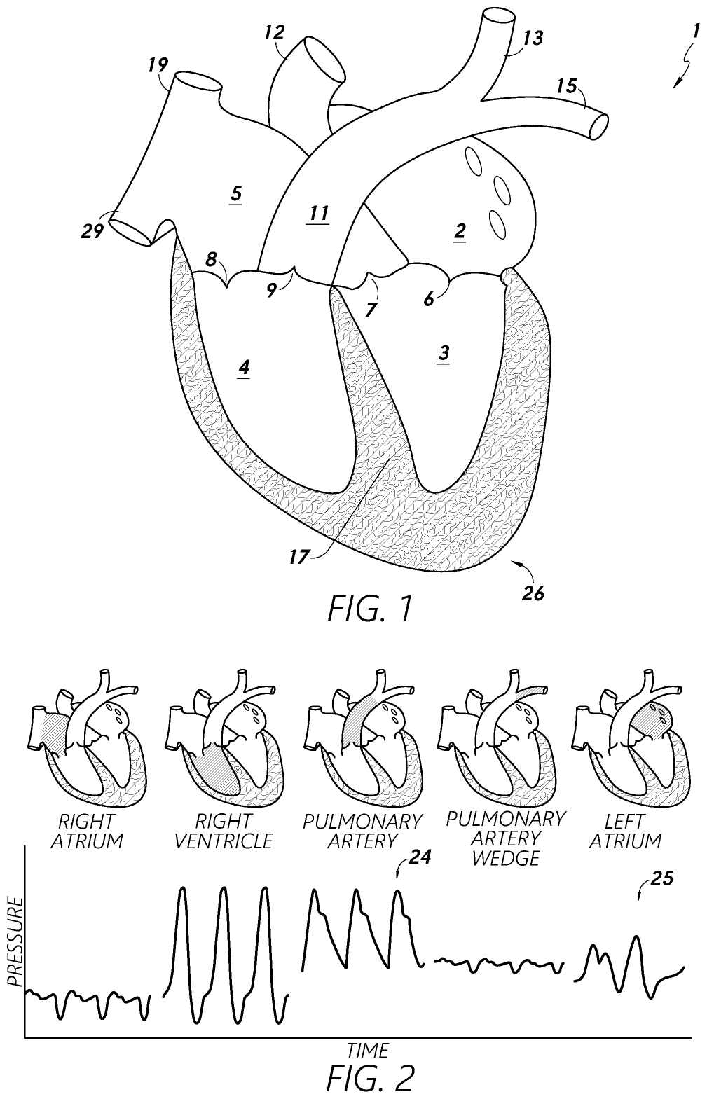

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can advantageously involve the monitoring of pressure in one or more chambers or regions of the heart or other anatomy. As described above, pressure buildup in one or more chambers or areas of the heart can be associated with congestive heart failure. Without direct or indirect monitoring of cardiac pressure, it can be difficult to infer, determine, or predict the presence or occurrence of congestive heart failure. For example, treatments or approaches not involving direct or indirect pressure monitoring may involve measuring or observing other present physiological conditions of the patient, such as measuring body weight, thoracic impedance, right heart catheterization, or the like. In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, F/A ratio determination generally does not provide absolute pressure measurement values.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. In addition, for patients that have been discharged, such treatments may necessitate remote telemedicine systems.

The present disclosure provides systems, devices, and methods for guiding the administration of medication relating to the treatment of congestive heart failure at least in part by directly monitoring pressure in the left atrium, or other chamber or vessel for which pressure measurements are indicative of left atrial pressure, in congestive heart failure patients in order to reduce hospital readmissions, morbidity, and/or otherwise improve the health prospects of the patient.

### Cardiac Pressure Monitoring

Cardiac pressure monitoring in accordance with embodiments of the present disclosure may provide a proactive intervention mechanism for preventing or treating congestive heart failure. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with embodiments of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to embodiments of the present disclosure, normal ventricular filling pressures may advantageously be maintained, thereby preventing or reducing effects of heart failure, such as dyspnea.

As referenced above, with respect to cardiac pressures, pressure elevation in the left atrium may be particularly correlated with heart failure. FIG. 2 illustrates example pressure waveforms associated with various chambers and vessels of the heart according to one or more embodiments. The various waveforms illustrated in FIG. 2 may represent waveforms obtained using right heart catheterization to advance one or more pressure sensors to the respective illustrated and labeled chambers or vessels of the heart. As illustrated in FIG. 2, the waveform 25, which represents left atrial pressure, may be considered to provide the best feedback for early detection of congestive heart failure. Furthermore, there may generally be a relatively strong correlation between increases and left atrial pressure and pulmonary congestion.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 24, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

Left atrial pressure readings may be relatively less likely to be distorted or affected by other conditions, such as respiratory conditions or the like, compared to the other pressure waveforms shown in FIG. 2. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with embodiments of the present disclosure may be used to predict heart failure up two weeks or more before the manifestation of symptoms or markers of heart failure (e.g., dyspnea). When heart failure predictors are recognized using cardiac pressure sensor embodiments in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

Implant Devices with Integrated Sensors

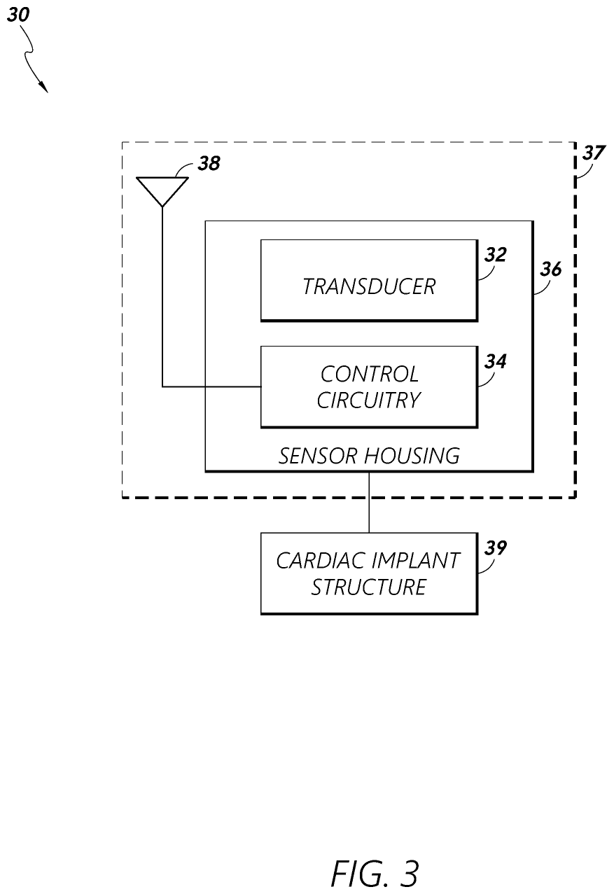

In some implementations, the present disclosure relates to sensors associated or integrated with cardiac shunts or other implant devices. Such integrated devices may be used to provide controlled and/or more effective therapies for treating and preventing heart failure and/or other health complications related to cardiac function. FIG. 3 is a block diagram illustrating an implant device 30 comprising a shunt (or other type of implant) structure 39. In some embodiments, the shunt structure 39 is physically integrated with and/or connected to a sensor device 37. The sensor device 37 may be, for example, a pressure sensor, or other type of sensor. In some embodiments, the sensor 37 comprises a transducer 32, such as a pressure transducer, as well as certain control circuitry 34, which may be embodied in, for example, an application-specific integrated circuit (ASIC).

The control circuitry 34 may be configured to process signals received from the transducer 32 and/or communicate signals associated therewith wirelessly through biological tissue using the antenna 38. The antenna 38 may comprise one or more coils or loops of conductive material, such as copper wire or the like. In some embodiments, at least a portion of the transducer 32, control circuitry 34, and/or the antenna 38 are at least partially disposed or contained within a sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 37 to allow for transportation thereof through a catheter or other introducing means.

The transducer 32 may comprise any type of sensor means or mechanism. For example, the transducer 32 may be a force-collector-type pressure sensor. In some embodiments, the transducer 32 comprises a diaphragm, piston, bourdon tube, bellows, or other strain- or deflection-measuring component(s) to measure strain or deflection applied over an area/surface thereof. The transducer 32 may be associated with the housing 36, such that at least a portion thereof is contained within or attached to the housing 36. The term “associated with” is used herein according to its broad and ordinary meaning. With respect to sensor devices/components being “associated with” a stent or other implant structure, such terminology may refer to a sensor device or component being physically coupled, attached, or connected to, or integrated with, the implant structure.

In some embodiments, the transducer 32 comprises or is a component of a piezoresistive strain gauge, which may be configured to use a bonded or formed strain gauge to detect strain due to applied pressure, wherein resistance increases as pressure deforms the component/material. The transducer 32 may incorporate any type of material, including but not limited to silicon (e.g., monocrystalline), polysilicon thin film, bonded metal foil, thick film, silicon-on-sapphire, sputtered thin film, and/or the like.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon, and the like. In some embodiments, the transducer 32 comprises or is a component of an electromagnetic pressure sensor, which may be configured to measure the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer 32 comprises or is a component of a strain gauge. For example, a strain gauge embodiment may comprise a pressure sensitive element on or associated with an exposed surface of the transducer 32. In some embodiments, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

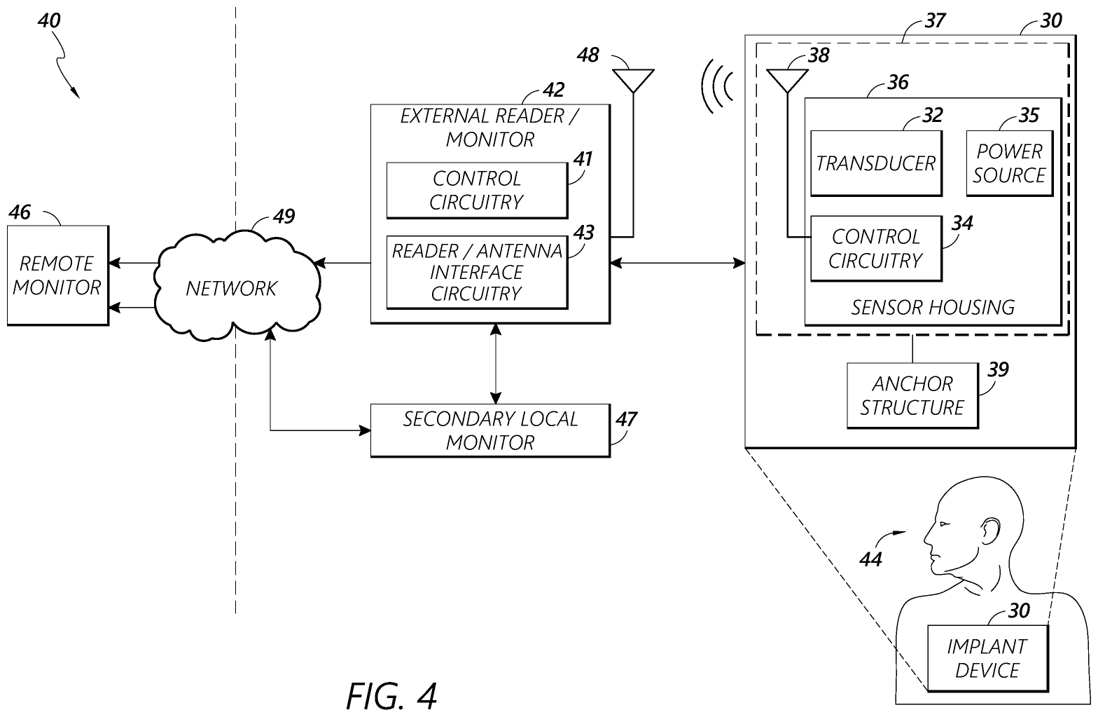

FIG. 4 shows a system 40 for monitoring one or more physiological parameters (e.g., left atrial pressure and/or volume) in a patient 44 according to one or more embodiments. The patient 44 can have a medical implant device 30 implanted in, for example, the heart (not shown), or associated physiology, of the patient 44. For example, the implant device 30 can be implanted at least partially within the left atrium of the patient's heart. The implant device 30 can include one or more sensor transducers 32, such as one or more microelectromechanical system (MEMS) devices, such as MEMS pressure sensors, or other type of sensor transducer.

In certain embodiments, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antennae coil). The monitoring system 40 can further include an external (e.g., non-implantable) subsystem that includes an external reader 42 (e.g., coil), which may include a wireless transceiver that is electrically and/or communicatively coupled to certain control circuitry. In certain embodiments, both the internal and external subsystems include a corresponding coil antenna for wireless communication and/or power delivery through patient tissue disposed therebetween. The sensor implant device 30 can be any type of implant device. For example, in some embodiments, the implant device 30 comprises a pressure sensor integrated with another functional implant structure, such as a prosthetic shunt or stent device/structure.

Certain details of the implant device 30 are illustrated in the enlarged block 30 shown. The implant device 30 can comprise a cardiac implant structure 39 as described herein. For example, the cardiac implant structure 39 can include a percutaneously-deliverable shunt device configured to be secured to and/or in a tissue wall to provide a flow path between two chambers and/or vessels of the heart, as described in greater detail throughout the present disclosure. Although certain components are illustrated in FIG. 4 as part of the implant device 30, it should be understood that the sensor implant device 30 may only comprise a subset of the illustrated components/modules and can comprise additional components/modules not illustrated. The implant device may represent an embodiment of the implant device shown in FIG. 3, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The sensor transducer(s) 32 can comprise one or more MEMS sensors, optical sensors, piezoelectric sensors, electromagnetic sensors, strain sensors/gauges, accelerometers, gyroscopes, diaphragm-based sensors, and/or other types of sensors, which can be positioned in the patient 44 to sense one or more parameters relevant to the health of the patient. The transducer 32 may be a force-collector-type pressure sensor. In some embodiments, the transducer 32 comprises a diaphragm, piston, bourdon tube, bellows, or other strain- or deflection-measuring component(s) to measure strain or deflection applied over an area/surface thereof. The transducer 32 may be associated with the sensor housing 36, such that at least a portion thereof is contained within, or attached to, the housing 36. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. With respect to sensor devices/components being “associated with” an implant structure, such terminology may refer to a sensor device or component being physically coupled, attached, or connected to, or integrated with, the implant structure.

In some embodiments, the transducer 32 comprises or is a component of a piezoresistive strain gauge, which may be configured to use a bonded or formed strain gauge to detect strain due to applied pressure, wherein resistance increases as pressure deforms the component/material. The transducer 32 may incorporate any type of material, including but not limited to silicone, polymer, silicon (e.g., monocrystalline), polysilicon thin film, bonded metal foil, thick film, silicon-on-sapphire, sputtered thin film, and/or the like. In some embodiments, the transducer 32 comprises or is a component of a strain gauge. In some embodiments, a metal strain gauge is adhered to the sensor surface, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicone, silicon or other semiconductor, and the like. In some embodiments, the transducer 32 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain embodiments, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and an antenna 38. The antenna 38 can comprise an internal antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some embodiments.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions and/or provide wireless power using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain embodiments, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain embodiments, the local monitor 42 comprises a smartphone, laptop computer, or other mobile computing device, or any other type of computing device.

In certain embodiments, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or another external subsystem. In certain embodiments, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain embodiments, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. In certain embodiments, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or another period of time).

In some embodiments, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain embodiments, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an embodiment, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain embodiments disclosed herein describe communication with the remote monitor subsystem 46 from the implant device indirectly through the local monitor device 42, in certain embodiments, the implant device 30 can comprise a transmitter capable of communicating over the network 49 with the remote monitor subsystem 46 without the necessity of relaying information through the local monitor device 42.

In certain embodiments, the antenna 48 of the external monitor system 42 comprises an external coil antenna that is matched and/or tuned to be inductively paired with the antenna 38 of the internal implant 30. In some embodiments, the implant device 30 is configured to receive wireless ultrasound power charging and/or data communication between from the external monitor system 42. As referenced above, the local external monitor 42 can comprise a wand or other hand-held reader.

In some embodiments, at least a portion of the transducer 32, control circuitry 34, power source 35 and/or the antenna 38 are at least partially disposed or contained within the sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 30 to allow for transportation thereof through a catheter or other percutaneous introducing means.

### Cardiac Shunt Implants

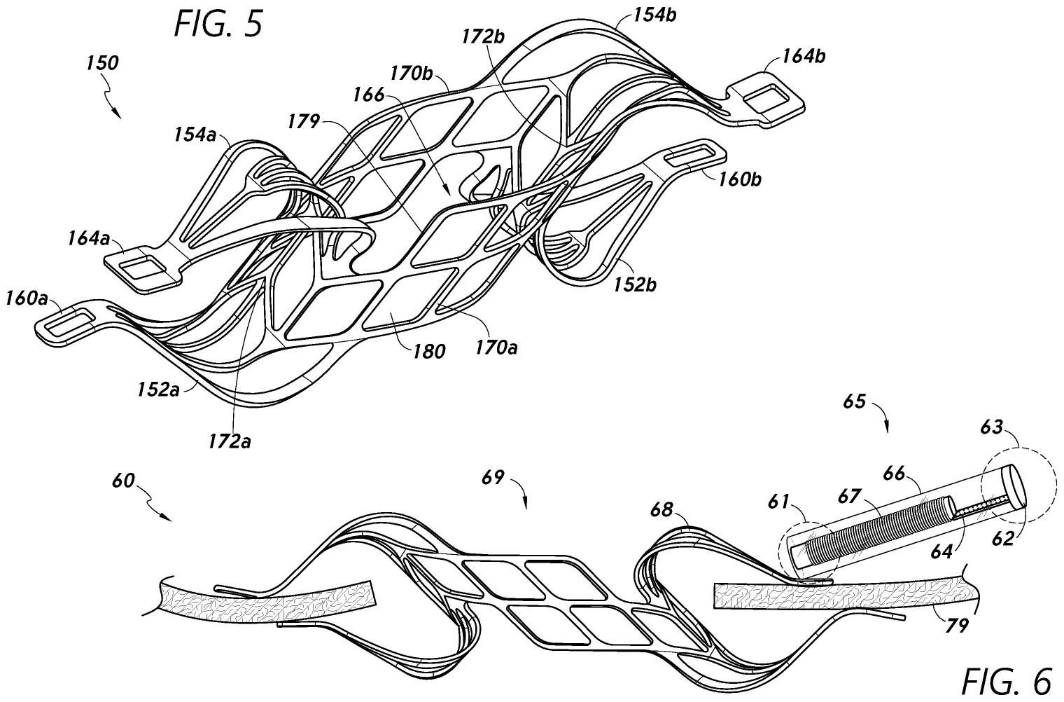

FIG. 5 illustrates an example shunt structure 150 in accordance with one or more embodiments. The shunt structure 150 may represent an embodiment of a cardiac implant device that may be integrated with pressure sensor functionality in accordance with certain embodiments disclosed herein. The shunt structure 150 may be an expandable shunt. When expanded, a central flow channel 166 of the shunt 150 may define a generally circular or oval opening. The channel 166 may be configured to hold the sides of a puncture opening in a tissue wall to form a blood flow path between chamber(s) or vessel(s) of the heart that are separated by the tissue wall. For example, the shunt 150 may be configured to be implanted in the wall separating the coronary sinus and the left atrium. The central flow channel 166 may be partly formed by a pair of side walls 170a, 170b defined by a generally parallelogram arrangement of thin struts 179 that forms an array of parallelogram-shaped cells or openings 180. In some embodiments, substantially the entire shunt 150 is formed by super-elastic struts that are configured to be compressed and fit into a catheter (not shown) and subsequently expanded back to the relaxed shape as shown in FIG. 5.

Formation of the shunt 150 using a plurality of interconnected struts forming cells therebetween may serve to at least partially increase the flexibility of the shunt, thereby enabling compression thereof and expansion at the implant site. The interconnected struts around the central flow channel 166 advantageously provide a cage having sufficient rigidity and structure to hold the tissue at the puncture in an open position. End walls 172a, 172b of the central flow channel 166 can serve to connect the side walls 170a, 170b and extend between distal and proximal flanges, or arms, 152, 154 on each side. The side walls 170a, 170b and end walls 172a, 172b together may define a tubular lattice, as shown. The end walls 172a, 172b can comprise thin struts 179 extending at a slight angle from a central flow axis of the shunt 150.

Although the illustrated shunt 150 comprises struts that define a tubular or circular lattice of open cells forming the central flow channel 166, in some embodiments, the structure that makes up the channel forms a substantially contiguous wall surface through at least a portion of the channel 166. In the illustrated embodiment, the tilt of the shunt structure 150 may facilitate collapse of the shunt into a delivery catheter (not shown), as well as the expansion of the flanges/arm 152, 154 on both sides of a target tissue wall. The central flow channel 166 may remain essentially unchanged between the collapsed and expanded states of the shunt 150, whereas the flanges/arms 152, 154 may transition in and out of alignment with the angled flow channel.

Although certain embodiments of shunts disclosed herein comprise flow channels having substantially circular cross-sections, in some embodiments, shunt structures in accordance with the present disclosure have oval-shaped, rectangular, diamond-shaped, or elliptical flow channel configuration. For example, relatively elongated side walls compared to the illustrated configuration of FIG. 5 may produce a rectangular or oval-shaped flow channel. Such shapes of shunt flow channels may be desirable for larger punctures, while still being configured to collapse down to a relatively small delivery profile.

In some embodiments, each of the distal and proximal flanges/arms 152, 154 is configured to curl outward from the end walls 172a, 172b and be set to point approximately radially away from the central flow channel 166 in the expanded configuration. The expanded flanges/arms may serve to secure the shunt 150 to a target tissue wall. Additional aspects and features of shunt structures that may be integrated with sensor devices/functionality in accordance with embodiments of the present disclosure are disclosed in U.S. Pat. No. 9,789,294, entitled “Expandable Cardiac Shunt,” issued on Oct. 17, 2017, the disclosure of which is hereby expressly incorporated by reference in its entirety. Although certain embodiments are disclosed herein in the context of shunt structures similar to that shown in FIG. 5 and described above, it should be understood that shunt structures or other implant devices integrated with pressure sensor functionality in accordance with embodiments of the present disclosure may have any type, form, structure, configuration, and/or may be used or configured to be used for any purpose, whether for shunting or other purpose or functionality.

Sensor-Retention Structures Integrated with Shunts and Other Implant Devices

Sensor devices in accordance with embodiments of the present disclosure may be integrated with cardiac shunt structures/devices or other implant devices using any suitable or desirable attachment or integration mechanism or configuration. FIG. 6 illustrates a sensor implant device 60 comprising a shunt structure 69 and an integrated sensor 65 in accordance with one or more embodiments. In some embodiments, the sensor 65 may be built or manufactured into the shunt structure 69 to form a unitary structure. In some embodiments, the sensor 65 may be attached to or integrated with an arm member 68 of the shunt structure 69.

The sensor 65 includes a sensor element 62, such as a pressure sensor transducer. Relative to the arm member 68 of the shunt structure 69, the transducer element 62 (e.g., pressure transducer) may be oriented/positioned at a distal 63 or proximal 61 end or area of the sensor 65. For example, the illustrated embodiment of FIG. 5 includes the transducer 62 disposed at the distal end 63 of the sensor 65. In some embodiments, readings acquired by the sensor may be used to guide titration of medication for treatment of a patient in whom the implant device 69 is implanted.

As described herein, the sensor 65 may be configured to implement wireless data and/or power transmission. The sensor 65 may include an antenna component 67 and control circuitry 64 configured to facilitate wireless data and/or power communication functionality. In some embodiments, the antenna 67 comprises one or more conductive coils, which may facilitate inductive powering and/or data transmission.

The sensor 65 may advantageously be biocompatible. For example, the sensor 65 may comprise a biocompatible housing 66, such as a cylindrical or other-shaped housing comprising glass or other biocompatible material. The circuitry 64, sensor element 62, and/or antenna 67 may be at least partially contained within the housing 66, wherein the housing 66 is sealed to prevent exposure to such components to the external environment. However, at least a portion of the sensor element 62, such as a diaphragm or other component, may be exposed to the external environment in some embodiments in order to allow for pressure readings, or other parameter sensing, to be implemented. The housing 66 may comprise an at least partially rigid cylindrical or tube-like form, such as a glass cylinder form, wherein the sensing probe 62 is disposed at one or both ends 61, 63 of the sensor assembly 65. In some embodiments, the sensor assembly is approximately 3 mm or less in diameter and/or approximately 20 mm or less in length. The sensor element 62 may comprise a pressure transducer, as described herein.

The sensor assembly 65 may be configured to communicate with an external system when implanted in a heart or other area of a patient's body. For example, the sensor 65 may receive power wirelessly from the external system and/or communicate sensed data or waveforms to and/or from the external system. The sensor assembly 65 may be attached to, or integrated with, the shunt structure 69 in any suitable or desirable way. For example, in some implementations, the sensor 65 may be attached or integrated with the shunt structure 69 using mechanical attachment means. In some embodiments, as described in detail below, the sensor assembly 65 may be contained in a pouch or other receptacle that is attached to the shunt structure 69.

The sensor element 62 may comprise a pressure transducer. For example, the pressure transducer may be a microelectromechanical system (MEMS) transducer comprising a semiconductor diaphragm component. In some embodiments, the transducer may include an at least partially flexible or compressible diaphragm component, which may be made from silicone or other flexible material. The diaphragm component may be configured to be flexed or compressed in response to changes in environmental pressure. The control circuitry 64 may be configured to process signals generated in response to said flexing/compression to provide pressure readings. In some embodiments, the diaphragm component is associated with a biocompatible layer on the outside surface thereof, such as silicon nitride (e.g., doped silicon nitride) or the like. The diaphragm component and/or other components of the pressure transducer 62 may advantageously be fused or otherwise sealed to/with the housing 66 in order to provide hermetic sealing of at least some of the sensor assembly components.

The control circuitry 64 may comprise one or more electronic application-specific integrated circuit (ASIC) chips or die, which may be programmed and/or customized or configured to perform monitoring functionality as described herein and/or facilitate transmission of sensor signals wirelessly. The antenna 67 may comprise a ferrite core wrapped with conductive material in the form of a plurality of coils (e.g., wire coil). In some embodiments, the coils comprise copper or other metal. The antenna 67 may advantageously be configured with coil geometry that does not result in substantial displacement or heating in the presence of magnetic resonance imaging. In some implementations, the sensor implant device 60 may be delivered to a target implant site using a delivery catheter (not shown), wherein the delivery catheter includes a cavity or channel configured to accommodate the advancement of the sensor assembly 65 therethrough.

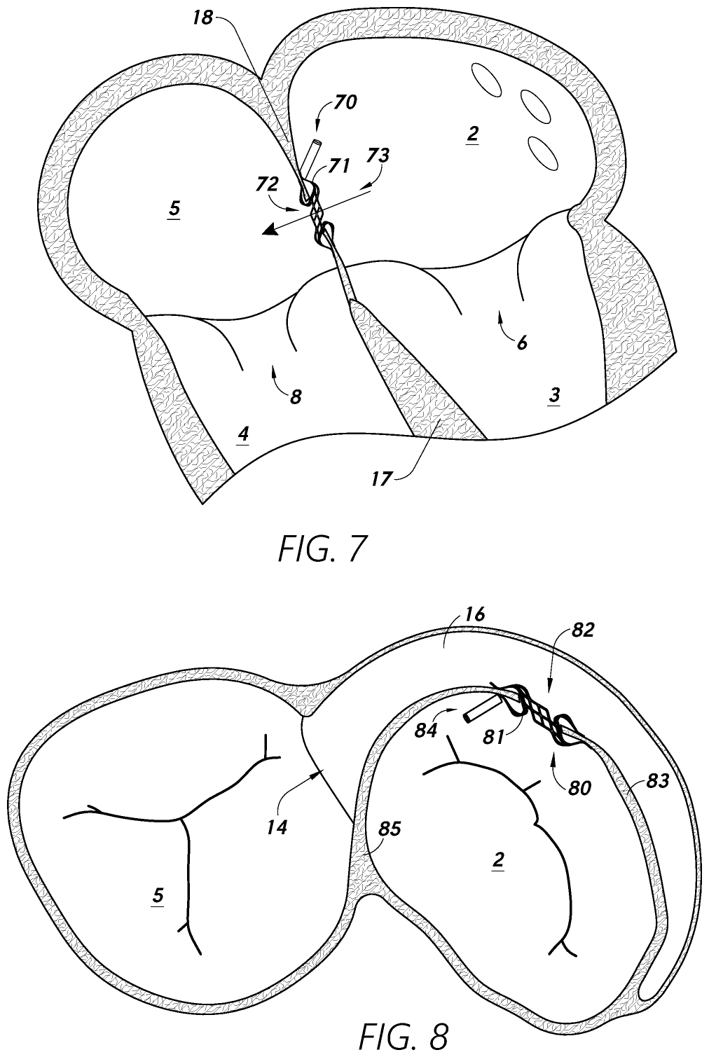

FIG. 7 shows a sensor implant device 73 implanted in an atrial septum 18 in accordance with one or more embodiments. The particular position in the interatrial septum wall may be selected or determined in order to provide a relatively secure anchor location for the shunt structure 72, as well as to provide a relatively low risk of thrombus. Furthermore, the sensor implant device 73 may be implanted at a position that is desirable in consideration of future re-crossing of the septal wall for future interventions. Implantation of the sensor implant device 73 in the interatrial septum wall may advantageously allow for communication between the left 2 and right 5 atria. With the device 73 in the atrial septum 18, the sensor 70 of the sensor implant device 73 may advantageously be configured to measure pressure in the right atrium 5, the left atrium 2, or both atria. For example, in some embodiments, the device 73 comprises a plurality of sensors, wherein one sensor is disposed in each of the right atrium 5 and the left atrium 2. With pressure sensor functionality for measuring pressure in both atria, the sensor implant device 73 may advantageously be configured to provide sensor signals that may be used to determine differential pressure between the atria. Differential pressure determination may be useful for monitoring fluid build-up in the lungs, which may be associated with congestive heart failure.

Interatrial shunting using the sensor implant device 73, which may integrate pressure-monitoring functionality in some embodiments, may advantageously be well-suited for patients that are relatively highly sensitive to atrial pressure increases. For example, as pressure increases in the ventricles and/or atria and is applied against the myocardial cells, the muscles of the heart may generally be prone to contract relatively harder according to process the excess blood. Therefore, as the ventricle dilates or stretches, for patients with compromised contractility of the ventricle, such patients may become more sensitive to higher pressures in the ventricle and/or atria because the heart may be unable to adequately respond or react thereto. Furthermore, increases in left atrial pressure can results in dyspnea, and therefore reduction in left atrial pressure to reduce dyspnea and/or reduce incidences of hospital readmission may be desirable through interatrial shunting. For example, when the ventricle experiences dysfunction such that is unable to accommodate build-up in fluid pressure, such fluid may backup into the atria, thereby increasing atrial pressure. With respect to heart failure, minimization of left ventricular end-diastolic pressure may be paramount. Because left ventricular end-diastolic pressure can be related to left atrial pressure, backup of fluid in the atrium can cause backup of fluid in the lungs, thereby causing undesirable and/or dangerous fluid buildup in the lungs. Interatrial shunting, such as using shunt devices in accordance with embodiments of the present disclosure, can divert extra fluid in the left atrium to the right atrium, which may be able to accommodate the additional fluid due to the relatively high compliance in the right atrium.

In some situations, interatrial shunting may not be sufficiently effective due to the patient being subject to a drug regimen designed to control the patient's fluid output and/or pressure. For example, diuretic medications may be used to cause the patient to expel excess fluid. Therefore, use of pressure-sensor-integrated implants in accordance with embodiments of the present disclosure may provide a mechanism to inform technicians or doctors/surgeons with respect to how to titrate such medications to adjust/modify fluid status. Therefore, embodiments of the present disclosure may advantageously serve to direct medication intervention to reduce or prevent the undesirable increase in left atrial pressure.

In some implementations, sensor-integrated shunt implant devices in accordance with embodiments of the present disclosure may be implanted in the wall separating the coronary sinus from the left atrium. For example, interatrial shunting may be achieved through the coronary sinus. FIG. 8 shows a sensor implant device 80 implanted in a tissue wall 83 between the coronary sinus 16 and the left atrium 2. FIG. 8, as well as a number of the following figures, shows a section of the heart from a top-down perspective with the posterior aspect oriented at the top of the page.

Interatrial shunting through implantation of the shunt device 80 in the wall 83 between the left atrium 2 and the coronary sinus 16 can be preferable to shunting through the interatrial septum 85 in some situations. For example, shunting through the coronary sinus 16 can provide reduced risk of thrombus and embolism. The coronary sinus is less likely to have thrombus/emboli present for several reasons. First, the blood draining from the coronary vasculature into the right atrium has just passed through capillaries, so it is essentially filtered blood. Second, the ostium of the coronary sinus in the right atrium is often partially covered by a pseudo-valve called the Thebesian Valve. The Thebesian Valve is not always present, but some studies show it is present in most hearts and can block thrombus or other emboli from entering in the event of a spike in right atrium pressure. Third, the pressure gradient between the coronary sinus and the right atrium into which it drains is generally relatively low, such that thrombus or other emboli in the right atrium is likely to remain there. Fourth, in the event that thrombus/emboli do enter the coronary sinus, there will be a much greater gradient between the right atrium and the coronary vasculature than between the right atrium and the left atrium. Most likely, thrombus/emboli would travel further down the coronary vasculature until right atrium pressure returned to normal and then the emboli would return directly to the right atrium.

Some additional advantages to locating the shunt structure 82 between the left atrium and the coronary sinus is that this anatomy is generally more stable than the interatrial septal tissue. By diverting left atrial blood into the coronary sinus, sinus pressures may increase by a small amount. This would cause blood in the coronary vasculature to travel more slowly through the heart, increasing perfusion and oxygen transfer, which would be more efficient and also could help a dying heart muscle to recover.

In addition to the above-mentioned benefits, by implanting the shunt device 80 in the wall of the coronary sinus 83, damage to the interatrial septum 85 may be prevented. Therefore, the interatrial septum may be preserved for later transseptal access for alternate therapies. The preservation of transseptal access may be advantageous for various reasons. For example, heart failure patients often have a number of other comorbidities, such as atrial fibrillation and/or mitral regurgitation; certain therapies for treating these conditions require a transseptal access.

It should be noted, that in addition to the various benefits of placing the implant 80 between the coronary sinus 16 and the left atrium 2, certain drawbacks may be considered. For example, by shunting blood from the left atrium 2 to the coronary sinus 16, oxygenated blood from the left atrium 2 may be passed to the right atrium 5 and/or non-oxygenated blood from the right atrium 5 may be passed to the left atrium 2, both of which may be undesirable with respect to proper functioning of the heart.

Access to the target wall 83 and left atrium 2 via the coronary sinus 16 may be achieved using any suitable or desirable procedure. For example, various access pathways may be utilized in maneuvering guidewires and catheters in and around the heart to deploy an expandable shunt integrated or associated with a pressure sensor in accordance with embodiments of the present disclosure. In some embodiments, access may be achieved through the subclavian or jugular veins into the superior vena cava (not shown), right atrium 5 and from there into the coronary sinus 16. Alternatively, the access path may start in the femoral vein and through the inferior vena cava (not shown) into the heart. Other access routes may also be used, each of which may typically utilize a percutaneous incision through which the guidewire and catheter are inserted into the vasculature, normally through a sealed introducer, and from there the system may be designed or configured to allow the physician to control the distal ends of the devices from outside the body.

In some embodiments of procedures for advancing implant devices in accordance with aspects of the present disclosure, a guidewire is introduced through the subclavian or jugular vein, through the superior vena cava and into the coronary sinus. Once the guidewire provides a path, an introducer sheath may be routed along the guidewire and into the patient's vasculature, typically with the use of a dilator. The delivery catheter may be advanced through the superior vena cava to the coronary sinus of the heart, wherein the introducer sheath may provide a hemostatic valve to prevent blood loss. In some embodiments, a deployment catheter may function to form and prepare an opening in the wall of the left atrium, and a separate placement or delivery catheter will be used for delivery of an expandable shunt. In other embodiments, the deployment catheter may be used as the both the puncture preparation and implant delivery catheter with full functionality. In the present application, the terms “deployment catheter” or “delivery catheter” are used to represent a catheter or introducer with one or both of these functions.

As illustrated in FIG. 8, the coronary sinus is generally contiguous around the left atrium 2, and therefore there are a variety of possible acceptable placements for the implant device 80 and/or shunt structure 82. The target site selected for placement of the shunt structure 82, may be made in an area where the tissue of the particular patient is less thick or less dense, as determined beforehand by non-invasive diagnostic means, such as a CT scan or radiographic technique, such as fluoroscopy or intravascular coronary echo (IVUS).

Additional aspects and features of processes for delivering shunt structures that may be integrated with sensor devices/functionality in accordance with embodiments of the present disclosure for implantation in the wall between the coronary sinus and the left atrium are disclosed in U.S. Pat. No. 9,789,294, entitled “Expandable Cardiac Shunt,” issued on Oct. 17, 2017, the disclosure of which is hereby expressly incorporated by reference in its entirety. Although the implant device 80 is shown in the left atrium/coronary sinus wall, the implant device 80 may be positioned between other cardiac chambers, such as between the pulmonary artery and right atrium.

### Sensor-Support/Retention Struts/Structures

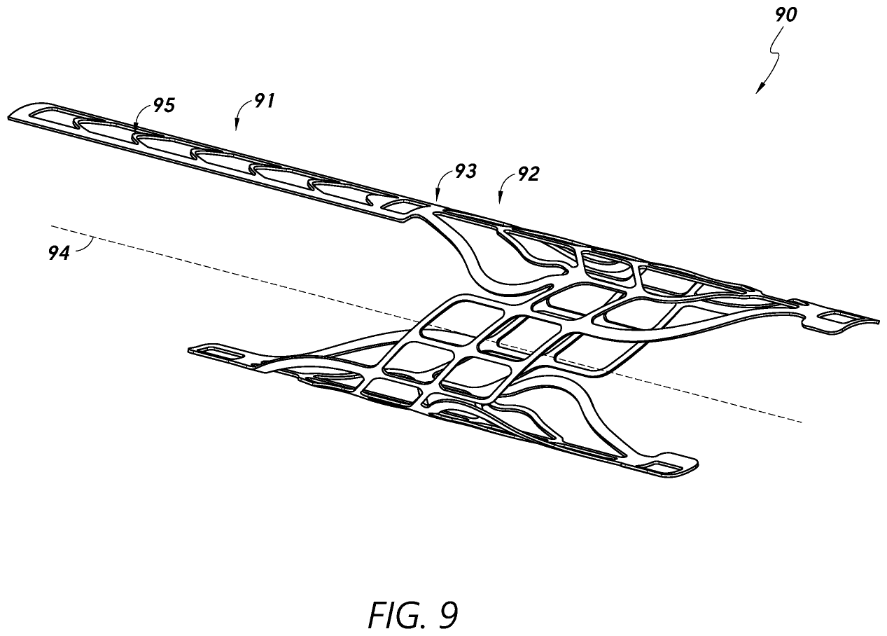

FIG. 9 shows a perspective view of a shunt device 90 in an at least partially collapsed configuration for delivery through a delivery sheath or catheter (not shown). The shunt device 90 includes a sensor-support structure/arm 91 attached or associated with an arm 92 of the shunt device.

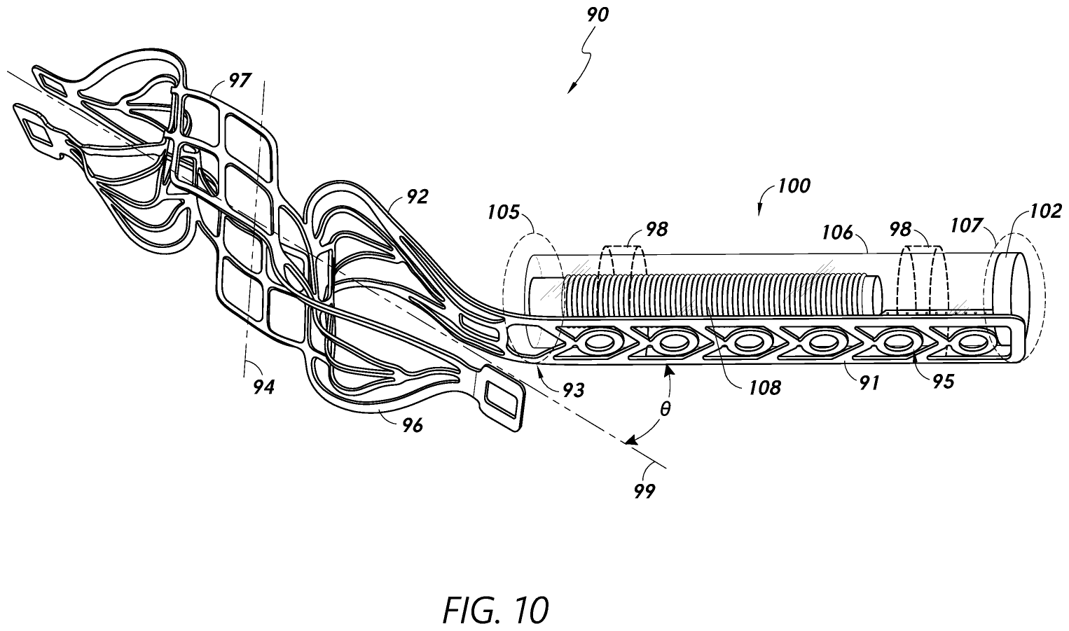

FIG. 10 illustrates a sensor implant device 90 having an integrated sensor 100 that is mechanically attached or fastened to a portion of a shunt structure 97. The shunt structure 97 comprises a sensor-support structure/arm 91, which may be a unitary form with the shunt structure 97. In some embodiments, the support 91 is an extension of, or otherwise associated with, an arm member 92 of the shunt structure 97. The sensor 100 may be attached to the support structure/arm 91 by any suitable or desirable attachment means, including adhesive attachment, or mechanical engagement. For example, the sensor support 91 may comprise or be associated with one or more retention features 98, which may comprise one or more clamps, straps, ties, sutures, collars, clips, tabs, or the like. Such retention features 98 may circumferentially encase or retain the sensor 100, or a portion thereof. In some embodiments, the sensor 100 may be attached to the sensor support 91 through the application of mechanical force, either through sliding the sensor 100 through the retention features 98 or through clipping, locking, or otherwise engaging the sensor 100 with the sensor support 91 by pressing or applying other mechanical force thereto. In some embodiments, the retention features 98 comprise one or more tabs that may be configured to pop-up or extend on one or more sides of the sensor support 91 for mechanical fastening. Such tabs may comprise memory metal (e.g., Nitinol) or other at least partially rigid material. In some embodiments, the sensor support 91 is at least partially non-rigid. For example, the sensor support 91 may comprise a non-rigid tether configured to float the sensor 100. Such configurations may advantageously allow for the sensor 100 to move with the blood flow.

In some embodiments, the sensor 100 is pre-attached to the sensor support 91 and/or integrated therewith prior to implantation. For example, in some embodiments, the sensor support 91 forms at least a portion of the housing of the sensor 100, such that the sensor support 91 and at least a portion of the housing of the sensor 100 are a unitary form.

In some embodiments, the angle or position of the sensor support 91 and/or sensor 100 relative to a longitudinal axis 99 of the shunt structure 97 is such that the sensor projects away from the longitudinal axis 99. For example, where the shunt structure 97 is engaged with biological tissue along the dimension of the longitudinal axis 99, the sensor 100 may advantageously project at least partially away from the biological tissue, such as into a chamber cavity (e.g., atrium of a heart). In some embodiments, the sensor support 91 is configured, or can be configured, substantially at a right angle or 90° orientation with respect to the axis 99, such that the sensor is substantially orthogonal to the longitudinal axis of the shunt. Such configurations may advantageously allow for the sensor element to be positioned a desirable distance away from the shunted flow flowing through the flow path axis 94.

The sensor element 102 of the sensor 100 may be disposed or positioned at any location of the sensor 100. For example, the sensor element 102 may advantageously be disposed at or near a distal portion 107 of the sensor 100. Alternatively or additionally, a sensor element may be disposed or positioned at or near a proximal portion 105 of the sensor 100.

The sensor device 100 can include electrical coupling component(s) 108, which may comprise, for example, one or more conductive (e.g., metal) coils. Such coils can be configured to inductively couple wirelessly to an external transmitter/receiver. The electrical coupling component(s) may have a magnetic core (e.g., iron; ferrite) to provide desirable magnetic permeability and/or electrical conductivity characteristics. The various embodiments disclosed herein provide sensor-retention structures configured to hold and/or retain certain sensor devices, which may be similar in one or more respects to the sensor device 100. Where such sensor-retention structures include conductive support arms (e.g., memory metal or other metal), such conductive features can cause interference with signals transmitted to/from the electrical coupling component(s) 108 when the conductive features of the sensor-retention structure axially overlap the electrical coupling component(s) 108. Therefore, it should be understood that the various sensor-retention features disclosed in connection with any of the embodiments of the present disclosure may be designed to reduce the degree to which conductive features thereof axially overlap the electrical/wireless coupling component(s) of the relevant sensor device. Furthermore, sensor devices used in connection with the various embodiments of sensor-retention structures disclosed herein may be configured such that the electrical coupling components thereof have a minimal amount of axial overlap with conductive features of the respective sensor-retention structures.

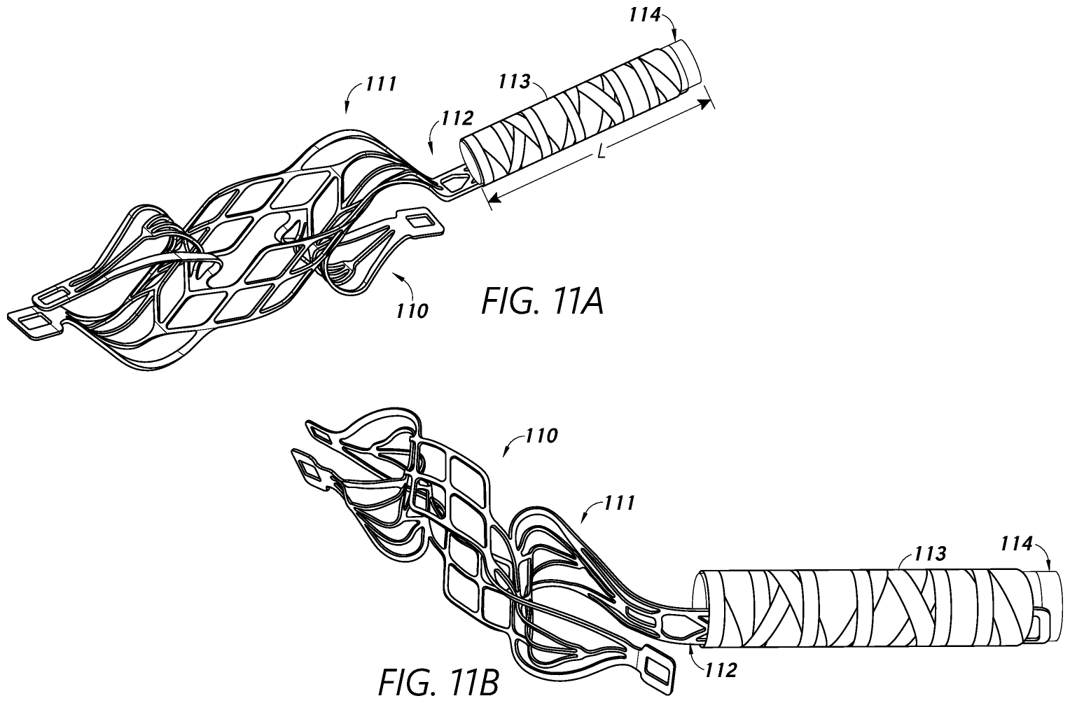

FIGS. 11A and 11B show perspective views of a medical implant device including a sensor-support strut/arm and a fabric and/or polymer cloth wrap configured to at least partially secure a sensor device to the sensor-support strut/arm. The polymer cloth wrap can include 0.003″ ePTFE biaxial membrane and/or certain stitching above and/or below the membrane (e.g., PET stitching). In some embodiments, the wrap 113 is wrapped in strips circumferentially and/or axially on the sensor cylinder and around the sensor-support strut 112.

The sock or wrap 113 can comprise polymer and/or fabric material, which may take the form of one or more strips of material that are wrapped around the cylinder/sensor 114 in a circumferential fashion traversing at least a portion of the length L of the cylinder 114. In some embodiments, the sock/wrap 113 has a sock-like form that is pulled or applied over the cylinder and the sensor-support strut/structure 112. For example, sutures or other type of line or stitching may be wrapped around the sock to secure the sock to the sensor 114 and the strut 112. Such suture/line may comprise ePTFE, PET, or the like. With respect to embodiments incorporating suture/line reinforcement (e.g., stitching), it may be desirable to protect such features from tissue in-growth. In some embodiments, suture/line reinforcement is omitted to protect against undesirable tissue in-growth.

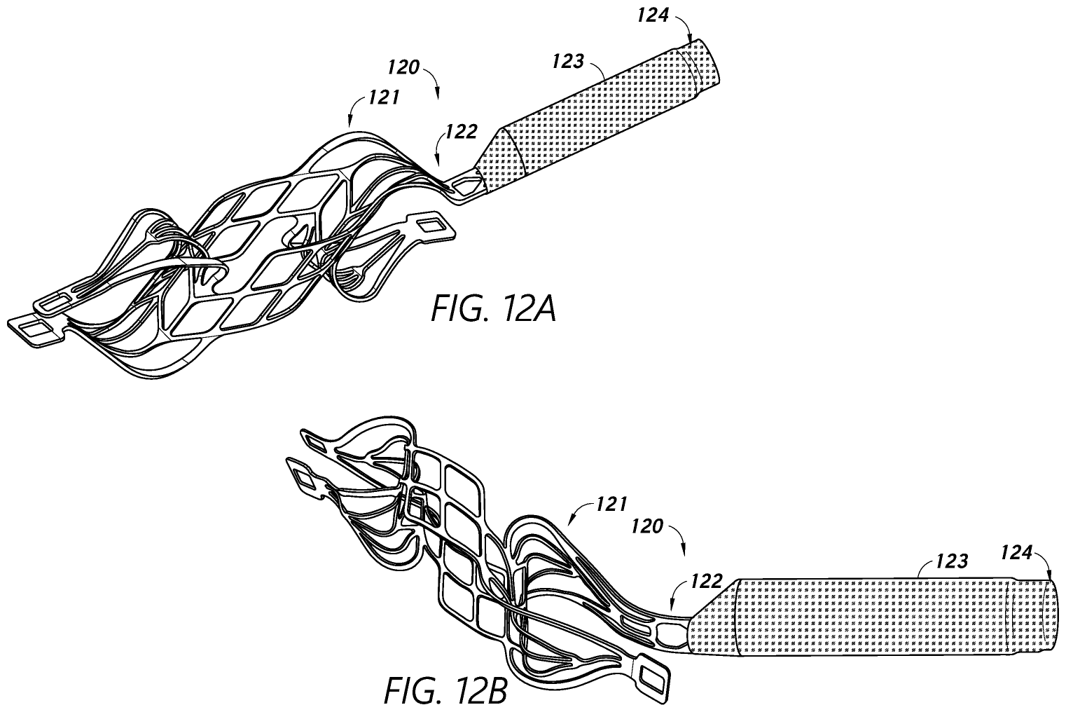

FIGS. 12A and 12B show perspective views of a medical implant device including a sensor-support structure or assembly 120 including a sensor-support strut/arm and a membrane sock- or wrap-type retention means or feature 123 configured to at least partially secure a sensor device to the sensor-support strut/arm. The membrane wrap can comprise polytetrafluoroethylene (PTFE) and/or polyurethane (PU) (e.g., electrospun or rotary-jet-spun) membrane. In some embodiments, the membrane 123 is wrapped in strips circumferentially and/or axially on the sensor cylinder and around the sensor-support strut 122 and a sensor device 124 that is supported/held by the sensor support structure 120.

In some embodiments, the membrane has certain heat and voltage characteristics with respect to application process(es) thereof that are such as to not result in undesirable effects/damage with respect to the sensor 124. Wraps, socks, sleeves, membranes, coatings, or similar types of features described herein in connection with the various disclosed embodiments may be applied to sensor-retention structures and/or sensors in any suitable or desirable manner. For example, such materials may be applied using electrospinning process(es) in some implementations. Certain methods, devices, and systems relating to electrospinning concepts that may be applicable to embodiments of the present disclosure are disclosed in U.S. Publication No. 2017/0325976, the disclosure of which is hereby incorporated by reference in its entirety. Electrospinning PTFE is described in U.S. Patent Publication No. 2010/0193999, which is incorporated herein by reference. Other processes that may be implemented to apply wrap, sock, sleeve, membrane, or similar features can include rotary jet spinning. Certain methods, devices, and systems relating to rotary jet spinning concepts that may be applicable to embodiments of the present disclosure are disclosed in U.S. Pat. No. 9,410,267, the disclosure of which is hereby incorporated by reference herein in its entirety.

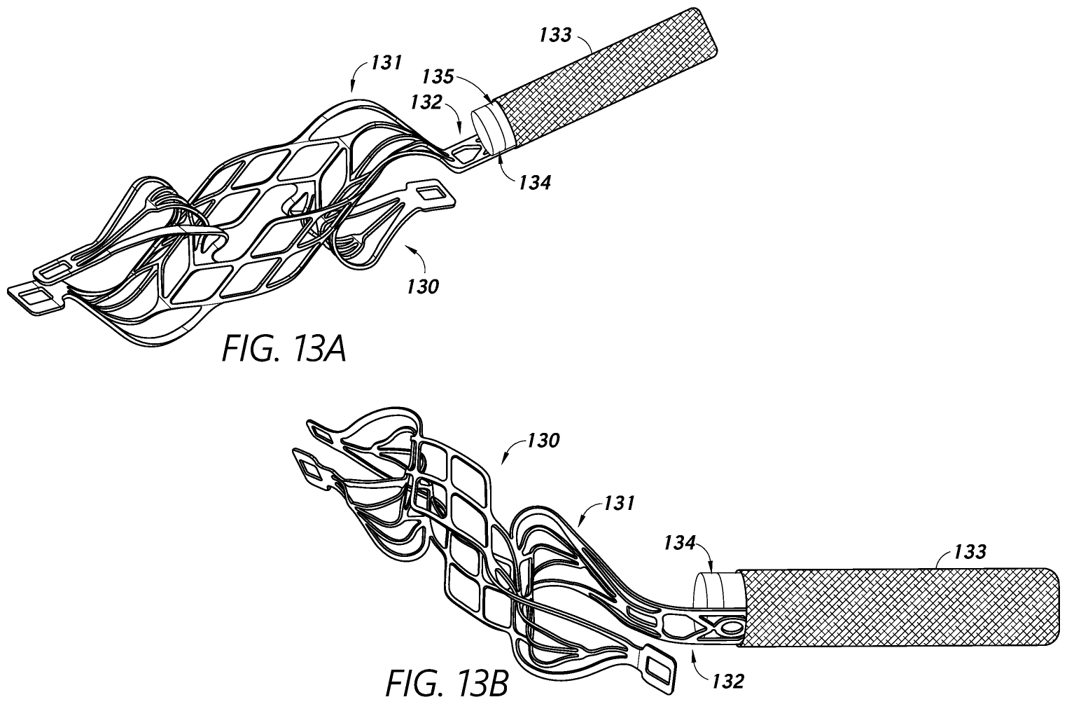

FIGS. 13A and 13B illustrate perspective views of a sensor implant device 130 having an integrated sensor 134 that is attached to a portion of a shunt structure 139 in accordance with one or more embodiments. The sensor 134 is attached to, or held within, a pouch or sock 133 that is attached to or otherwise associated with an arm/strut member 132 or another portion of the shunt structure 139 (e.g., anchor arm 131). For example, the pouch 133 may be a suture-based or cloth-based (e.g., fibrous and/or polymer cloth) pouch, wrapping, or other retention material and/or form.

The pouch 133 can comprise any suitable or desirable material, including polytetrafluoroethylene (PTFE), expanded polytetrafluoroethylene (ePTFE), polyurethane (PU), or the like and/or combinations of similar materials. Such material may be electrospun onto the sensor 135 in some implementations, or may be applied using rotary jet spinning.

In some embodiments, the sensor 134 is configured to be slidingly disposed within the pouch 133, wherein tension and/or compression of the pouch 133 serves to retain the sensor 134 in a fixed position within the pouch 133. Although a pouch/wrap is illustrated in FIGS. 13A and 13B that envelops at least a portion of the sensor 134 in a sock-/tube-like manner, in some embodiments, the pouch 133 comprises a band or other non-enveloping retention means. In some embodiments, the sensor 134 may be sutured or otherwise attached or fixed to the pouch 133. Furthermore, the pouch 133 may be sutured or otherwise fixed or attached to the arm member 132 of the shunt structure 139. The pouch 133 may advantageously be open on one or both of a distal and proximal end thereof to allow for fluid contact with a sensor element/transducer associated with the sensor 134. That is, the sensor 134 may be exposed through an open portion on a distal or proximal end of the strut 132 and/or pouch 133.

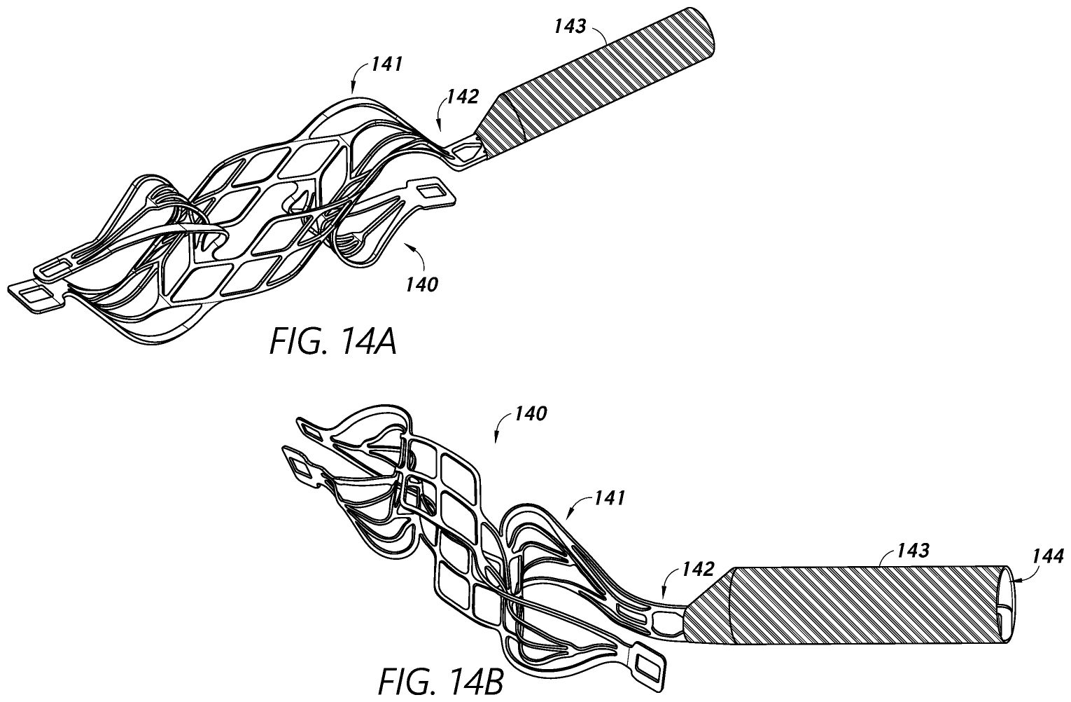

FIGS. 14A and 14B show perspective views of a medical implant device including a sensor-support strut/arm and a membrane wrap configured to at least partially secure a sensor device to the sensor-support strut/arm. The membrane wrap can comprise polytetrafluoroethylene (PTFE) and/or polyurethane (PU) (e.g., electrospun or rotary-jet-spun) membrane. In some embodiments, the membrane wrap 143 includes carbothane balloon and/or laser bonding. Use of a carbothane balloon, or similar, can provide desirable manufacturing and biocompatibility benefits in some cases.

Sensor-Retention Structures with Sensor-Retention Fingers and Other Features

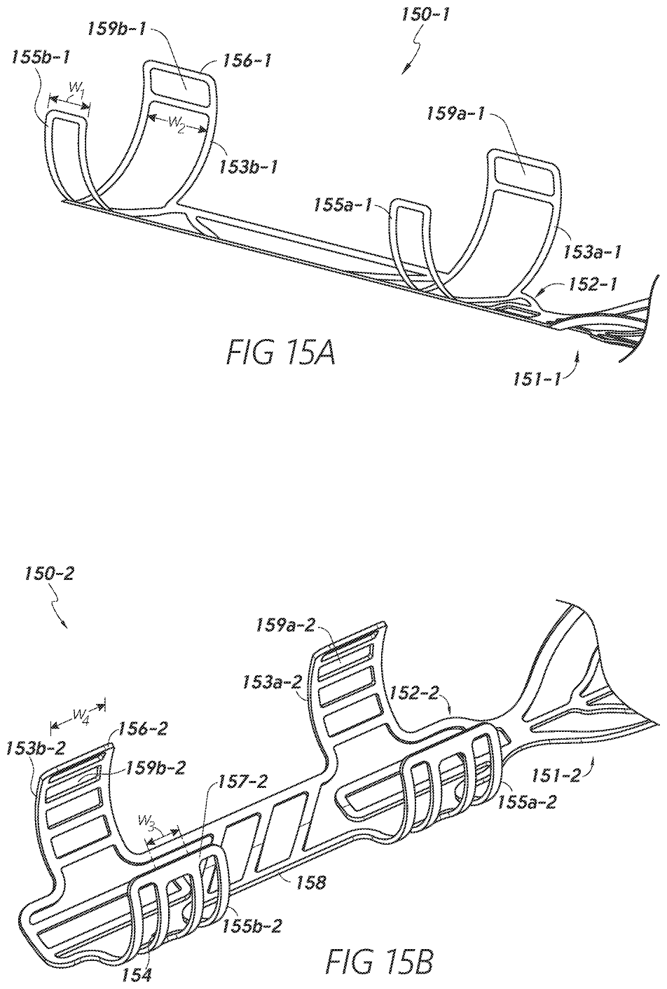

FIGS. 15A and 15B show perspective views of medical implant devices 150-1, 150-2 including respective sensor-support struts/arms 152-1, 152-2 and buckle and/or strap members 153-1, 153-2 and 155-1, 155-2 associated therewith. The buckle and/or strap members 153, 155 can be configured to be engaged with one another to secure a sensor device (not shown) to the sensor-support strut/arm 152.

With respect to FIG. 15A, the strap member 155b-1 (and/or 155a-1) may be shaped in an elongate, at least partially rectangular form having a width w₁ dimensioned to fit within an internal width w₂ of the coupling portion/aperture 159b-1 (and/or 159a-1) of the buckle member 153b-1 (and/or 153a-1), such that the coupling portion/aperture 159-1 can slide over a length of the strap member 155-1 to provide engagement therewith. For example, the strap 155-1 can be secured in a locked position within the coupling portion/aperture 159-1 by a friction and/or shape memory fit. Furthermore, the distal crossbar 156-1 can hold the strap 155-1 down and/or in place when the strap 155-1 is secured in the aperture 159-1.

With respect to FIG. 15B, the strap member 155b-2 (and/or 155a-2) may include a clasp 154 that is shaped in an elongate, at least partially rectangular form having a width us dimensioned to fit within an internal width w₄ of the coupling portion/aperture 159b-2 (and/or 159a-2) of the buckle member 153b-2 (and/or 153a-2), such that the coupling portion/aperture 159-2 can slide over a length of the clasp 154 to provide engagement therewith. The clasp member 154 may generally project and/or be oriented back toward the base 158 of the sensor-retention structure 150-2, such that in order to insert the clasp 154 into the aperture, the buckle member 153-2 may be brought over (or under) the strap member 155-2 far enough to allow the clasp 154 to be bent and passed into the aperture 159-2. The backwards projection/orientation of the clasp 154 can hold the buckle 153-2 and strap 155-2 together when the buckle and strap are drawn outward after engagement of the clasp 154 with the aperture 159-2. For example, the crossbar 156-2 of the buckle 153-2 may be held against the crossbar portions 157-2 of the strap member 155-2 and/or the clasp 154. The strap 155-2 can further be secured in a locked position within the coupling portion/aperture 159-2 by a friction and/or shape memory fit.

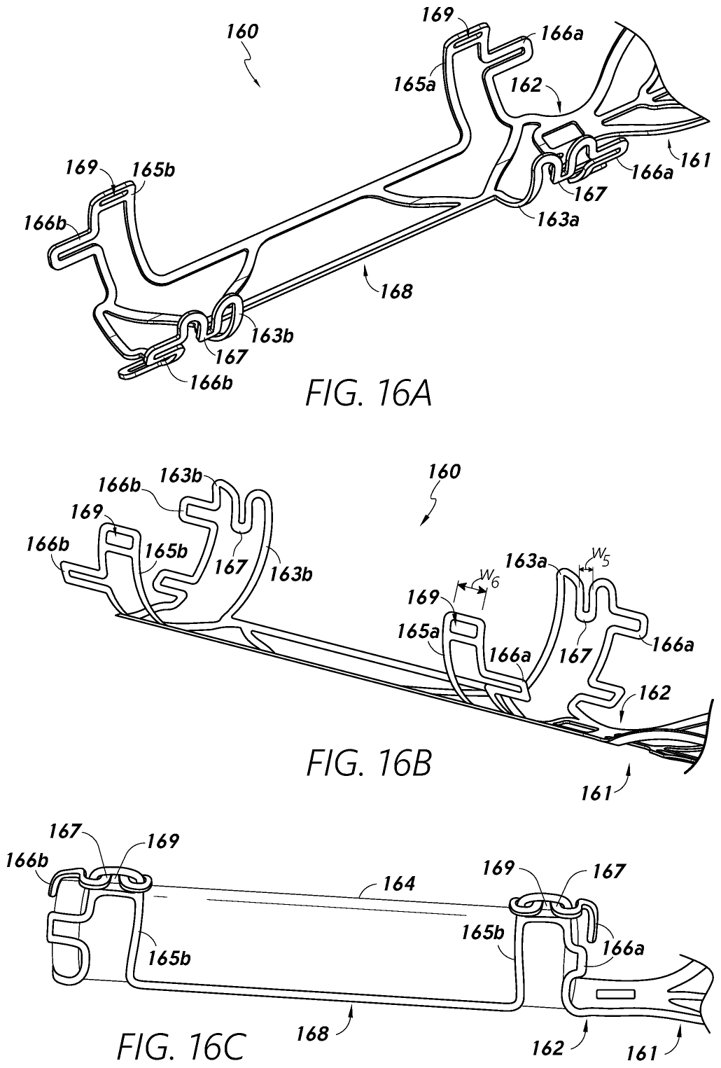

FIGS. 16A and 16B show perspective views of a medical implant device 160 including a sensor-support strut/arm 162 and one or more buckle and/or strap members 163, 165 associated therewith and/or one or more axial retention features 166. The buckle and/or strap members 163, 165 can be configured to be engaged with one another to secure a sensor device (not shown) to the sensor-support strut/arm 162. The one or more axial retention features may be configured to prevent a sensor device secured to the sensor-support strut/arm 162 from sliding proximally and/or axially when the features 166 are engaged as shown in FIG. 16C.

The strap members 163 may include one or more clasp features 167 that are shaped to form a tab-type form having a width w₅ dimensioned to fit within an internal width w₆ of a corresponding coupling portion/aperture 169 of the respective opposite-oriented buckle member 165, such that the coupling portion/aperture 169 can slide over the to provide engagement with the clasp 167. The clasp member 167 may generally project and/or be oriented back toward the base 168 of the sensor-retention structure 160 and/or base of the strap member 163, such that in order to insert the clasp 167 into the aperture 169, the buckle member 165 may be brought over (or under) the strap member 163 far enough to allow the clasp 167 to be bent and/or passed into the aperture 169. The backwards projection/orientation of the clasp 167 can hold the buckle 163 and strap 165 together when the buckle and strap are drawn outward after engagement of the clasp 167 with the aperture 169. For example, the distal crossbar of the buckle 165 may be held against the distal portions of the strap member 165 and/or the clasp 167. The strap 165 can further be secured in a locked position within the coupling portion/aperture 169 by a friction and/or shape memory fit.

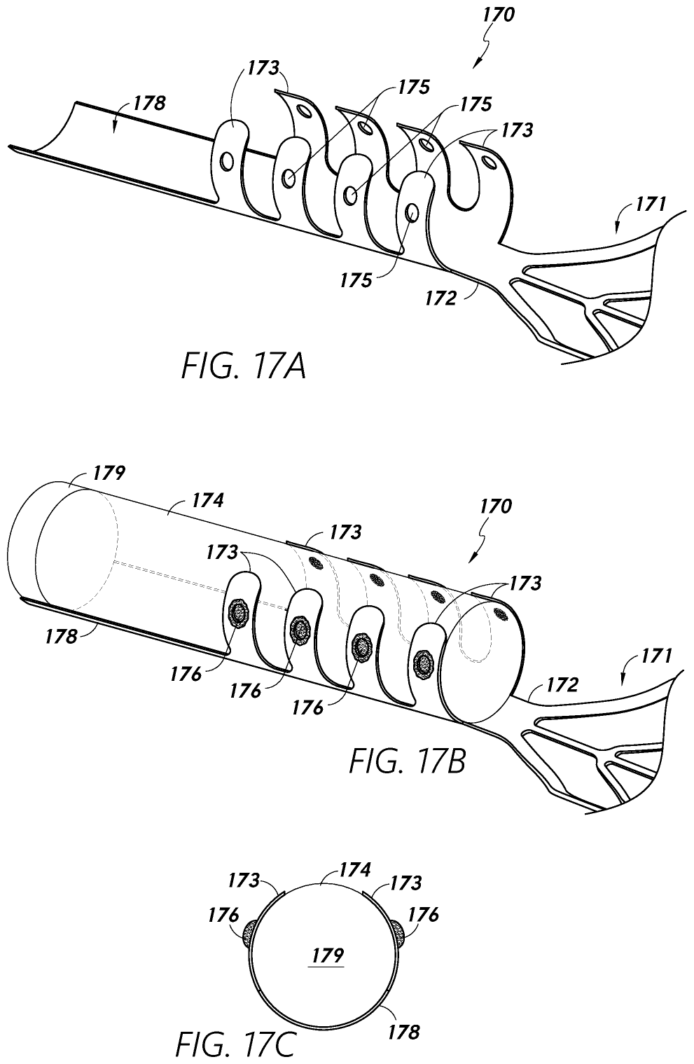

FIGS. 17A through 17C show perspective and end views, respectively of at least a portion of a medical implant device 170 including a sensor-retention structure 170 including sensor-support strut/arm 172 and one or more sensor-retention fingers 173 in accordance with one or more embodiments. The one or more sensor-retention fingers 173 can include aperture(s) 175 or other features configured to facilitate or allow for adhesive fixation of the finger(s) 173 to a sensor device disposed in contact with and/or in proximity to the finger(s) 173. FIG. 17B shows the sensor-retention structure 170 with a sensor device 174 retained at least in part thereby/therein, wherein adhesive material 176 is applied to/through the aperture features 175 to secure the finger(s) 173 to the sensor 174.

The adhesive 176 may be any suitable type of adhesive. The adhesive 176 is advantageously biocompatible to facilitate implantation of the implant device in the body. Although four fingers 173 are shown, each with an adhesive-engagement aperture/feature 175, it should be understood that the structure 170 can have any number of fingers and/or adhesive-engagement features. Furthermore, although closed apertures are shown as the adhesive-engagement features 175 in FIGS. 17A and 17B, it should be understood that adhesive-engagement features implemented in connection with any of the embodiments of the present disclosure can be open, closed, and/or have any other suitable or desirable shape or form. In some implementations, the adhesive 176 is applied to the fingers 173 and the sensor 174 on outside portions of the finger(s) 173. That is, no adhesive-engagement apertures may be present; rather, adhesive may be applied over the finger(s) 173 at or near a contact point between the respective finger(s) and the sensor 174. For example, the adhesive 176 may be applied on an underside of the finger(s) 173, such that the adhesive is disposed between the finger(s) 173 and the sensor 174. Additionally or alternatively, the adhesive 176 may be applied to the side(s) of the finger(s) 173 and contact the sensor 174.

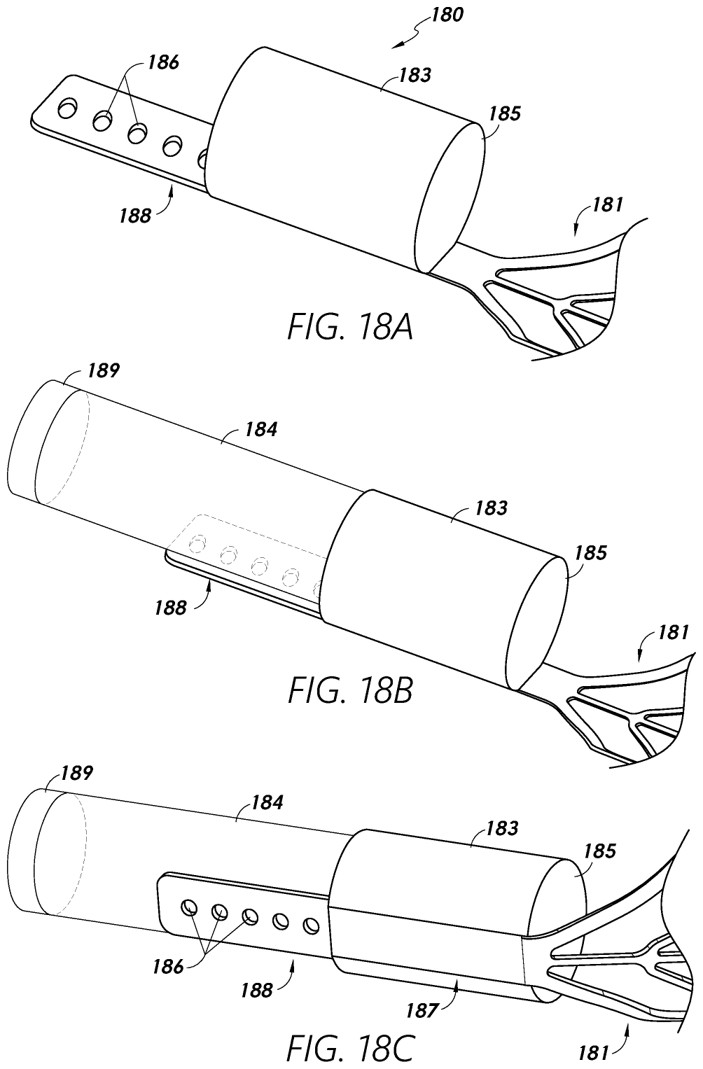

FIGS. 18A through 18C show perspective views of at least a portion of a medical implant device 180 including a sensor-support strut/arm 188 and a sensor-retention over-mold support form 183 in accordance with one or more embodiments. The strut/arm 188 can include aperture(s) 186 or other features configured to facilitate or allow for adhesive fixation of the strut 188 to a sensor device 184 disposed in contact with and/or in proximity to the strut 188. In some embodiments, the aperture(s) 186 and/or shape of the arm 188 facilitate coupling with the over-mold support form 183. FIG. 18B shows the medical implant device 180 with a sensor device 184 retained at least in part thereby, wherein the sensor 184 is nestingly disposed at least partially within the over-mold support form 183.

The over-mold support form 183 may be rigid or flexible. In some embodiments, the over-mold is bonded to the sensor 184 and/or support strut 188 through heat setting or other process. The sensor may be inserted into the over-mold support form 183, or the over-mold support form 183 may be applied over the sensor 184 and the support strut 188 after placement of the sensor 184 on the support strut. Although shown with apertures 186, it should be understood that the support strut may not have such apertures and may have any suitable or desirable shape, form, and/or configuration.

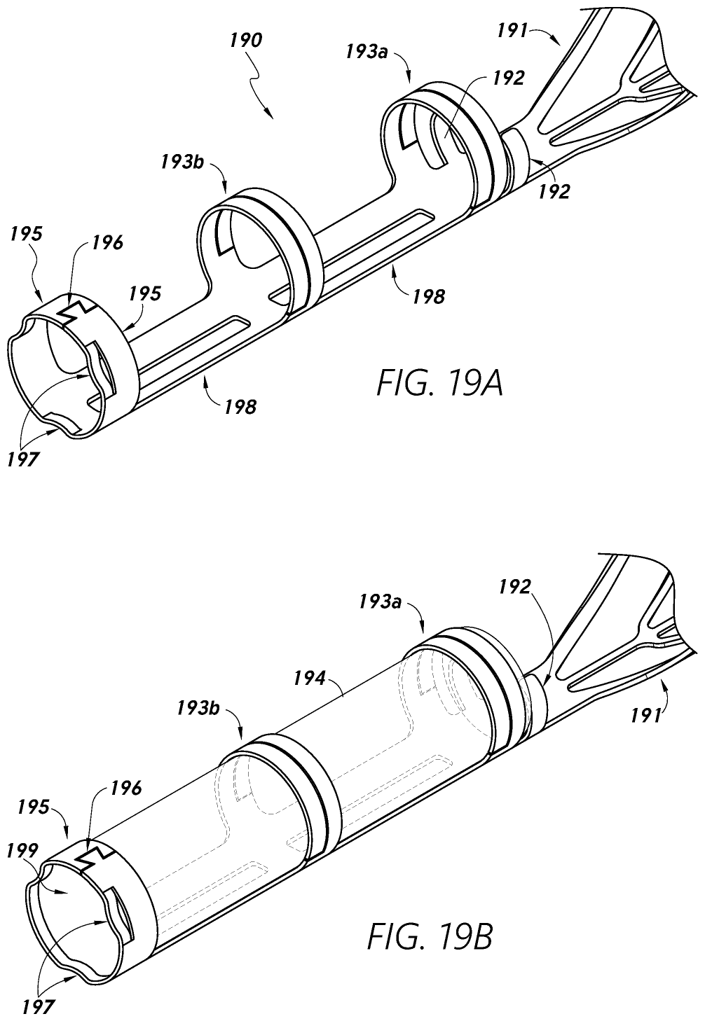

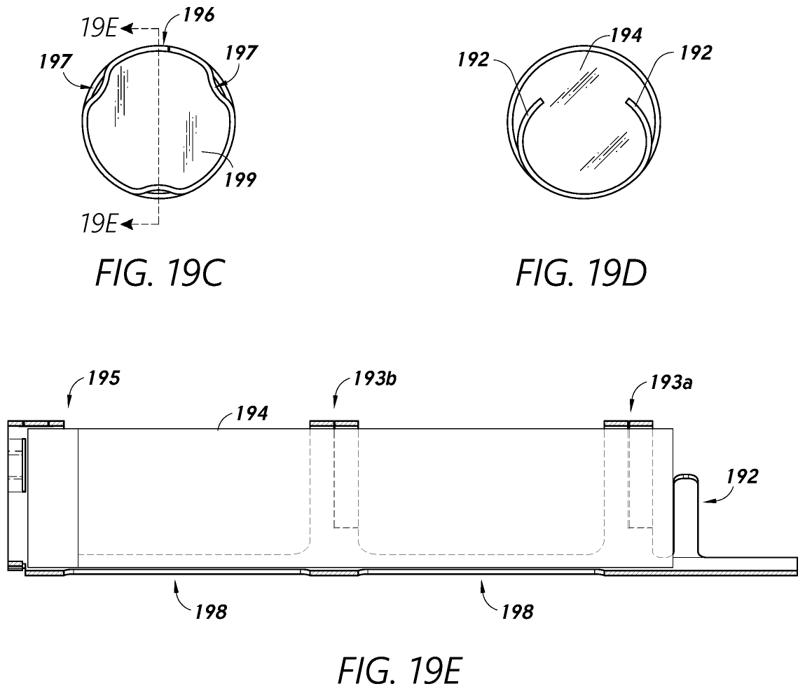

FIGS. 19A through 19E show at least a portion of a medical implant device 190 including a sensor-support strut/arm 198 associated with a plurality of sensor retention fingers 193, 195, at least two of which are mechanically-locking fingers 195 that can be locked using certain locking features 196, which may be configured to mechanically mate in some manner, as shown. The sensor-support strut/arm 198 can further include distal 197 and/or proximal 192 axial retention features for further securing a sensor 194 (see FIGS. 19C-19E) in a desired position.

The axial restraints/retention features 197, 192 can have any suitable or desirable configuration. In some embodiments, the distal axial retention features 197 include shape-set, inwardly-projecting tabs, which can have an at least partially curved/contoured shape corresponding to an inwardly-bent circumferential arc portion of a cylindrical form. With reference to FIG. 19C, which shows a distal end view of the implant device 190 with the sensor device 199 retained thereto/therein, the axial retention features 197 (also referred to as “eyebrow” features) may radially overlap at least a portion of a circumferential/perimeter region of the sensor device/transducer component 199. The overlapped portion of the sensor 199 may advantageously not cover a sensing membrane of the sensor device/transducer 199. That is, the radial overlap of the sensor 199 by the axial retention feature(s) 197 may not substantially affect the sensing functionality of the sensor 194 and/or sensor element 199 thereof.

The proximal retention features 192 can comprise one or more tabs/fingers that are configured to be bent or otherwise deflect or curl inwards towards an axis of the sensor 194. The radial overlap of the tabs/fingers 192 can serve to prevent proximal sliding of the sensor 194 past the tabs/fingers 192. The proximal retention features 192 may be shape set to a size that is smaller with respect to a radius of curvature thereof than a radius of curvature of the outer cylindrical form of the sensor 194 and/or the curvature of the fingers 193 that are wrapped at least partially around the body of the sensor 194.

The intermediate sensor-retention fingers are positioned axially between the distal 197 and proximal 192 axial retention features and are configured to prevent the sensor 194 from being drawn away from the sensor-support strut 198 when wrapped at least partially around the cylindrical body of the sensor 194. The fingers 193 may be relatively long compared to the fingers 192, and may be laser-cut in some embodiments.

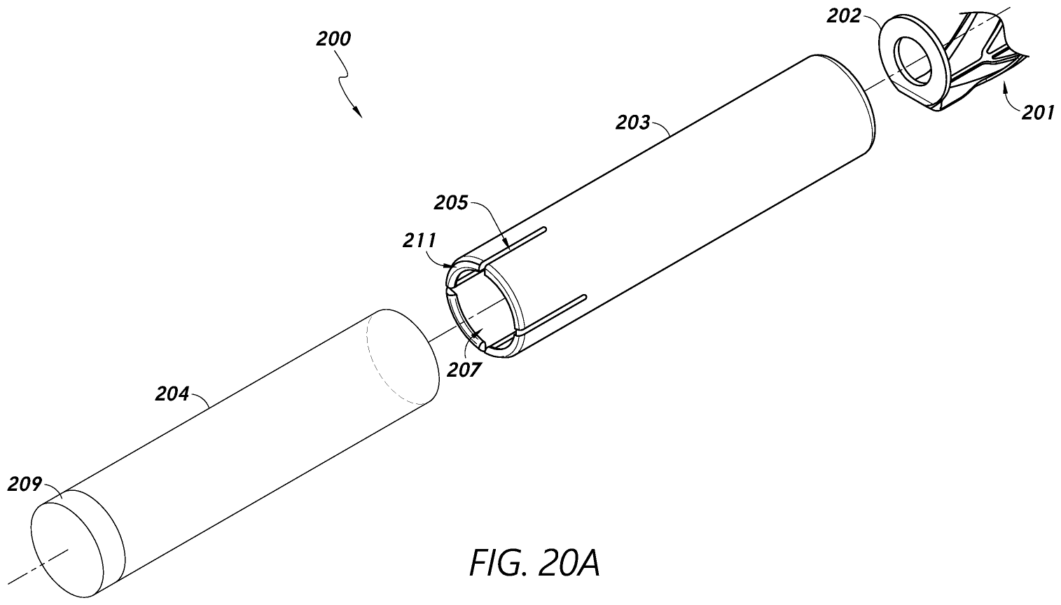

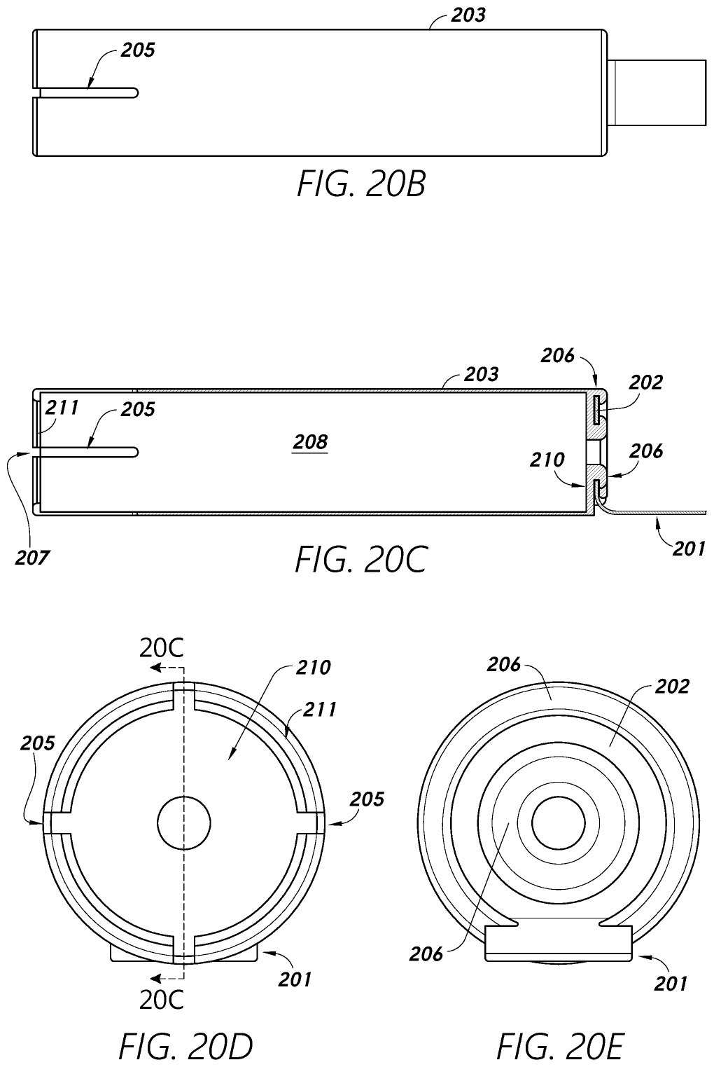

FIGS. 20A through 20E show at least a portion of a medical implant device 200 including an arm structure 201 associated with a housing-attachment flange configured to be attached to a sensor-retention housing 203 that is configured to hold a sensor device 204. The sensor-retention housing 203 can further include distal slit features configured to facilitate insertion of the sensor device 204 therein. The flange feature 202 can be shape-set and mechanically locked to the housing 203, which may comprise polyether ether ketone (PEEK) or other thermoplastic resin(s) and/or polymer(s).

In some embodiments, PEEK is used for the sensor-retention housing 203 due to certain properties thereof that may be desirable for use in sensor-retention embodiments and features disclosed herein. For example, PEEK can be considered a relatively inert material and may inhibit pannus growth thereon. Furthermore, PEEK can be relatively easy to machine, while providing sufficient stiffness and structural form that allows for manufacturing of relatively thin walls, which may be advantageous with respect to sensor-retention features due to the ability to form such structures without unduly increasing the profile of the sensor-retention device. For example, the walls of portions of PEEK sensor-retention features disclosed herein may be as thin as 0.005″. The sensor-retention housing 203 may be formed through a machining process in some implementations, or may be re-flowed.

The proximal housing-retention ring/flange 202 can be configured to snap/clip into the corresponding mating recess/clip feature 206. In some embodiments, embodiments, the proximal housing/ring coupling feature 206 of the sensor-retention housing 203 can be engaged with the housing-retention ring 202, wherein after such engagement, the feature 206 may be melted or otherwise wrapped over the ring 202 to create the mechanical bond/coupling therewith. That is, the material of the housing 203 may be re-flowed over the ring 202 to create a bond/coupling therewith.

The housing 203 can include distal slots 205 that allow the circumference of the housing 203 to expand outward in order to receive the sensor device 204 therein. In some embodiments, distal ridges/shoulder features 211 of the housing 203 can serve to axially retain the sensor 204 in the housing 203 once the sensor 204 has been inserted into the lumen/cavity of the housing 203.

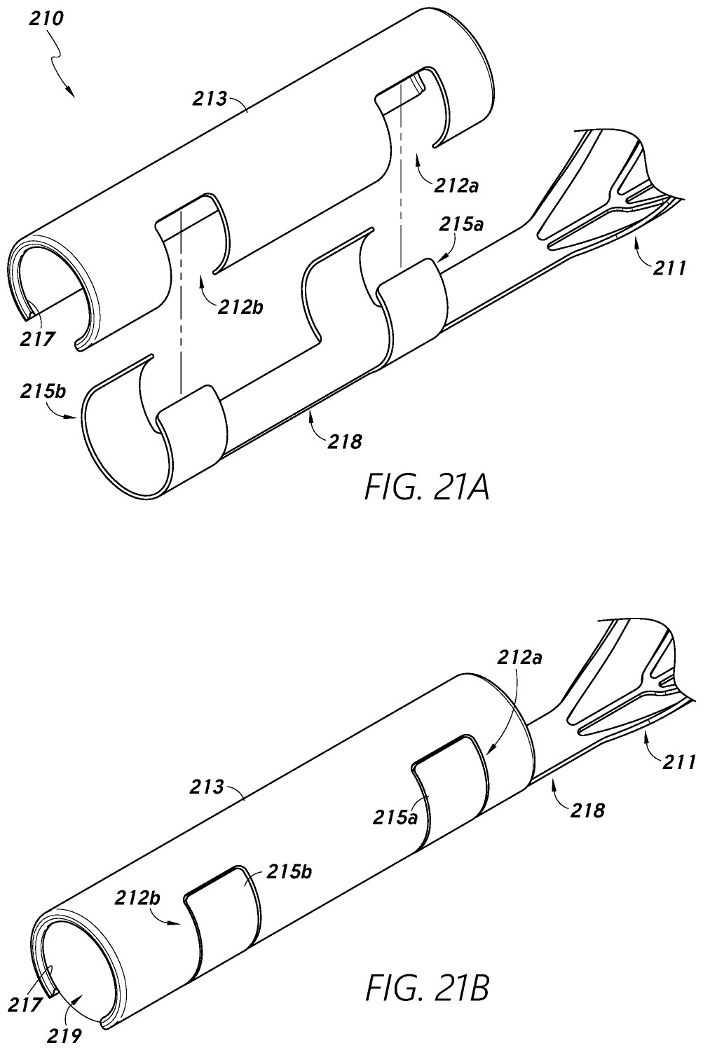

FIGS. 21A and 21B show at least a portion of a medical implant device 210 including an arm structure 211 associated with one or more sets of sensor-retention and/or housing-connection fingers 215. The fingers 215 are configured to engage and/or fit with corresponding features 212 of a sensor-retention cover/housing 213. A sensor device 219 can advantageously be contained at least partially within the cover 213 and an at least partially concave cradle arm portion 218.

In some embodiments, the sensor device 219 can be attached to the arm 218, after which the cover 213 can be snapped/disposed over the sensor cylinder 219. In some embodiments, the sensor 219 may be placed within the cover 213, wherein the combined sensor 219 and cover 213 may then be placed onto the arm 218. The fingers 215 may advantageously wrap around only a circumferential portion of the sensor, such that the opposing fingers do not touch when implanted, thereby providing a gap between opposing fingers through which the sensor 219 may be pressed/inserted.

The cover 213 can include distal ridges/shoulder features 217 that can serve to axially retain the sensor 219 in the cover 213 once the sensor 219 has been covered by the cover 213. The fingers 215 can fit between the axial/circumferential gaps 212 in the cover 213, wherein such engagement between the fingers 215 and gaps 212 can prevent axial movement of the cover 213 relative to the arm 218.

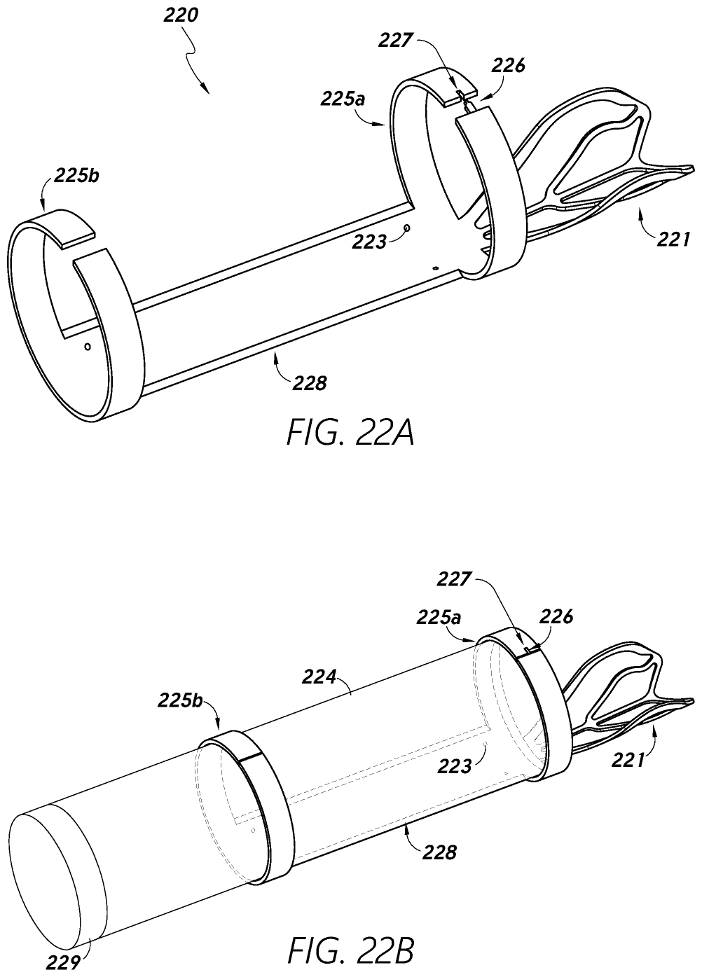

FIGS. 22A and 22B show at least a portion of a medical implant device 220 including a sensor-support strut/arm 228 associated with a plurality of sensor retention fingers 225, at least two of which are mechanically-locking fingers that can be locked using certain locking features 226, 227. The sensor-support strut/arm 228 can further include certain mating features (e.g., holes) 223 for mating with corresponding features (not shown) of a sensor device 224 to further secure the sensor device 224.

The mating features 223 can be configured and dimensioned to receive at least partially therein corresponding projections associated with the sensor cylinder 224. With the features 223 and corresponding projections/features of the sensor 224 engaged/mated together, the axial movement of the sensor 224 within the retention fingers 225 may be restrained thereby. In some embodiments, an adhesive layer may be applied to and/or between the sensor 224 and/or the arm 228 to secure the sensor 224 to the arm 228. Such adhesive may further at least partially fill the features 223 of the arm 228. Furthermore, any of the embodiments herein may include adhesive between sensor-retention arm components (e.g., metal components) and sensors retained thereby.

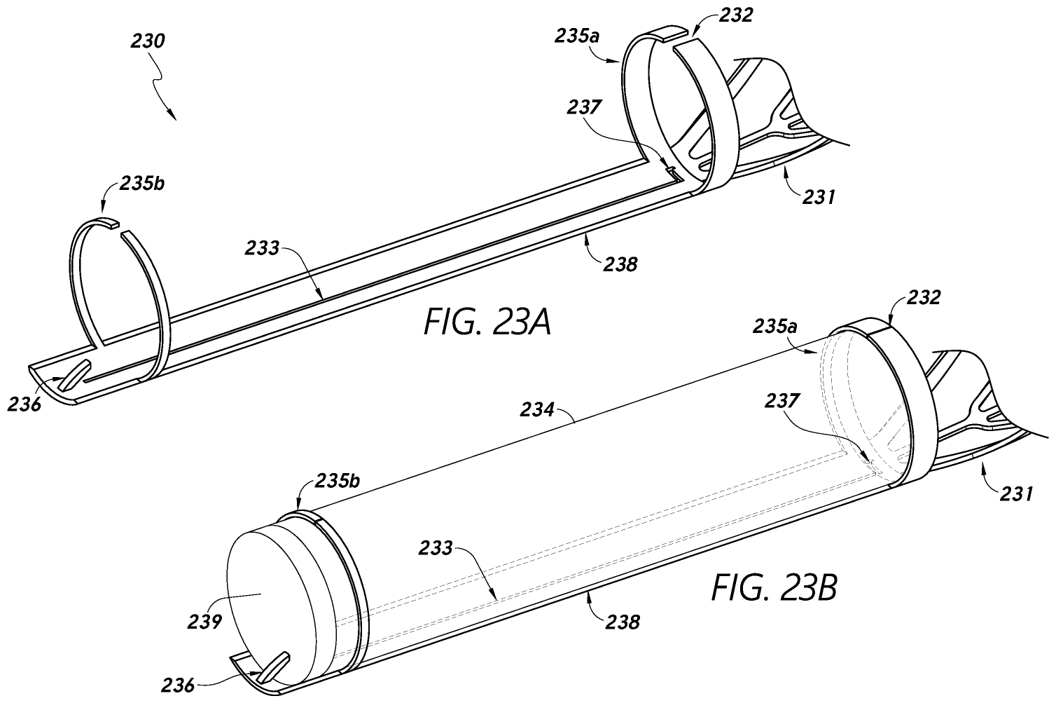

FIGS. 23A and 23B show at least a portion of a medical implant device 230 including a sensor-support strut/arm 238 associated with a plurality of sets of sensor retention fingers 235, wherein the various sets may or may not have different widths. In some embodiments, the implant device 230 includes one or more axial retention features at a distal and/or proximal end of the sensor-support strut/arm 238, such as one or more stopping tabs 236, or the like. The sensor-support strut/arm 238 can further include certain mating features (e.g., groove(s)) 233 for mating with corresponding features (not shown) of a sensor device 234 to further secure the sensor device 234. For example, the device 230 can include one or more longitudinal laser-cut grooves that key into and/or lock with one or more corresponding features (e.g., glass projection(s)) associated with the sensor device 234.

A process for securing the sensor 234 in the sensor-retention structure 230 may involve inserting a peg/projection component of the sensor 234 into the longitudinal groove 233 of the arm 238. The peg/projection may be configured to fit within the groove 233. In some embodiments, the peg/projection of the sensor (e.g., glass or metal projection from one or more portions of the sensor 234) may be disposed at or near a distal or proximal end of the sensor. Once the peg/projection reaches the circumferential notch 237, the sensor may be rotated to nest the peg/projection in the notch to restrain axial movement of the sensor 234. Although the notch 237 is shown in FIGS. 23A and 23B as being at a proximal end of the arm 238, it should be understood that the notch 237 may be at the distal end of the arm 238, or at any other point along the length of the arm 238. Furthermore, the stopping tab 236 may be at the distal end of the arm 238, as shown, thereby preventing distal sliding of the sensor 234, or at the proximal end of the arm 238, thereby preventing proximal sliding of the sensor 234. The notch 237 may have a longitudinal hook/turn at the end thereof to allow the peg/projection of the sensor 234 to be locked in place therein.

The placement of the tab 236 at the distal end can be undesirable because it may come into contact with the sensor membrane 239, thereby potentially corrupting sensor signals and/or damaging the sensor element. Therefore, embodiments of the present disclosure including such distal stopping tabs may advantageously be dimensioned and/or configured to present a sensor-contact surface that is relatively small and/or positioned to have a reduced impact on the structure/integrity and/or function of the sensor element 239. The fingers 235b may be shape-set and may or may not have any locking/mating features associated therewith.

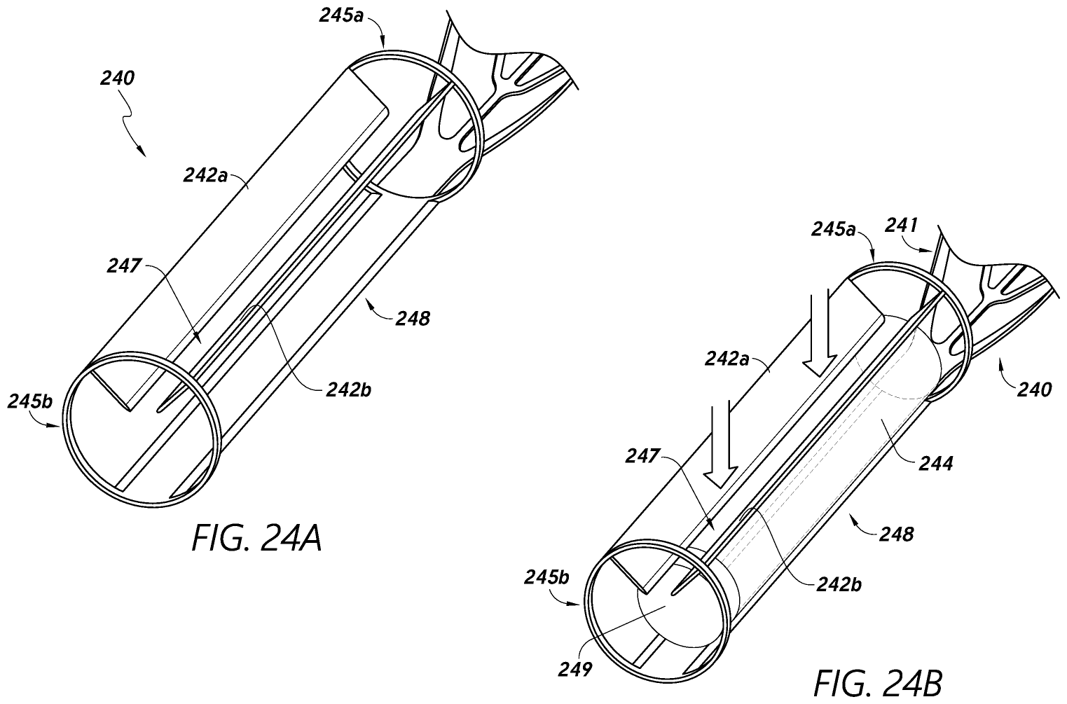

FIGS. 24A and 24B show perspective views of at least a portion of a medical implant device 240 including a sensor-support structure 248 and a trapdoor sensor-retention feature comprising one or more flaps or other similar components 242a. In some implementations, the sensor device 244 may be secured at least in part in the sensor retention arm 240 by passing or dropping the sensor 244 through a gap 247 in the trapdoor feature (e.g., between flaps 242a, 242b), which may allow for opening inward in the direction of the arm structure 248, whereas, once passed therethrough, the flaps 242a, 242b may at least partially prevent the sensor 244 from passing back through the trapdoor feature.

The sensor-support structure 248 can include rings 245 to which the flaps 242 may be secured in some manner. For example, the flaps 242 may be coupled to the rings 245 in a rotatable manner. In some embodiments, pegs or other features of the flaps/doors 242 are present/formed at side portions thereof that contact the respective rings 245. Such features may allow for hinging movement of the flaps 242. For example, the pegs/features of the flaps 242 may fit within corresponding holes or grooves in the rings 245 positioned at portions thereof where the flaps 242 contact the rings 245.

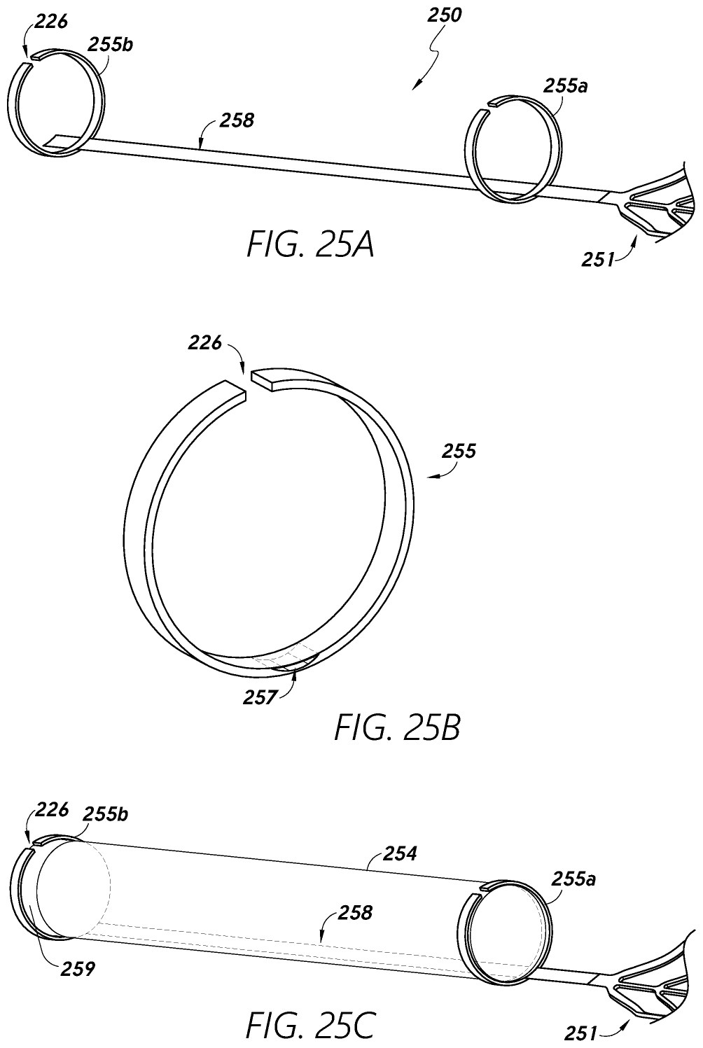

FIGS. 25A through 25C illustrate perspective views of a medical implant device 250 including a sensor-support arm 258 and one or more tension-fit rings 255. The rings 255 may be broken and/or include a gap 226 associated therewith, wherein such gap may advantageously allow for insertion and/or tensioning/securing of a sensor device in the rings 255. In some embodiments, the rings 255 may be secured or attached to the arm 258 by sliding, attaching, securing, clipping, or otherwise associating the arm 258 to/with a slot-type feature 257 of the rings 255.

The rings 255 may be positioned at any desirable spot along the arm 258. In some embodiments, the arm 258 includes features that hold the rings 255 in place, such as recesses, seatings, ridges, or the like. In some embodiments, the rings 255 may be able to slide relatively freely along the arm 258. A tension fit between the slot-type features 257 and the arm 258 may hold the rings 255 in place once they have been slid/moved to the desired positions along the length of the arm 258. In some embodiments, the shape setting of the rings 255 (e.g., memory metal rings) may introduce forces against the arm 258 that serve to hold the rings in place.

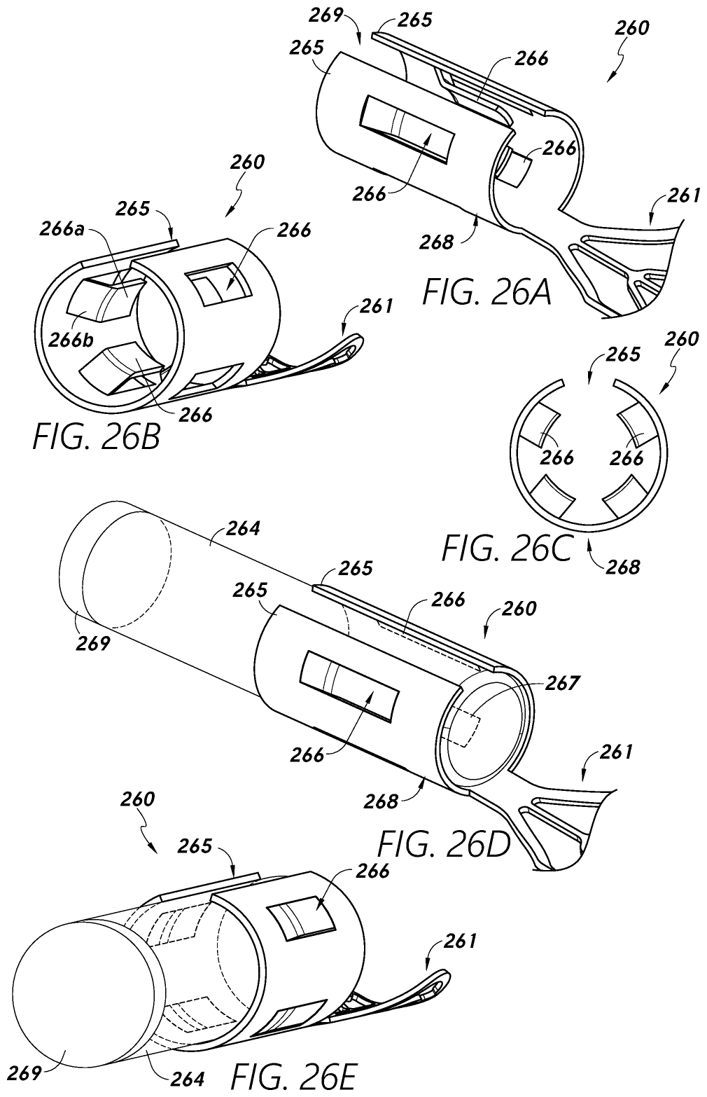

FIGS. 26A through 26E illustrate perspective views of a medical implant device 260 including a sensor-support arm 268 and one or more clamps features 265. In some embodiments, the clamp features 265 include inward-deflecting retention tabs 266, which are configured to present tension and/or pressure on a sensor device disposed therein.

The retention tabs 266 may have any suitable or desirable form, and may be laser-cut, or otherwise cut, from the barrel or sheet that forms the clamp features 265. The tab(s) 266 may provide a compressive friction retention functionality with respect to the sensor 264. The inward deflection of the tabs 266 may be achieved using shape memory setting in some implementations. Although the clamp features 266 are shown as having a gap 269 present between the clamp features 266 when in the sensor-retention configuration shown in FIGS. 26A-26E, in some embodiments, the edges of the clamp features 266 may come into contact with one another, or into closer proximity than is shown. The clamp features 266 advantageously can wrap around most of a circumferential area of the sensor cylinder 264 to thereby impede movement of the sensor 264 in the direction away from the base 268 of the retention structure 260.

In the deflected configuration shown, the tabs may have an axially flat sensor-contact portion 266a and a deflected portion 266b. the sensor-contact portion may provide surface contact area for providing friction hold with the sensor 264, wherein the deflected portion 266b provide inward force on the sensor-contact portion 266b to maintain surface contact and increase the friction between the sensor-contact portion 266a and the sensor 264.

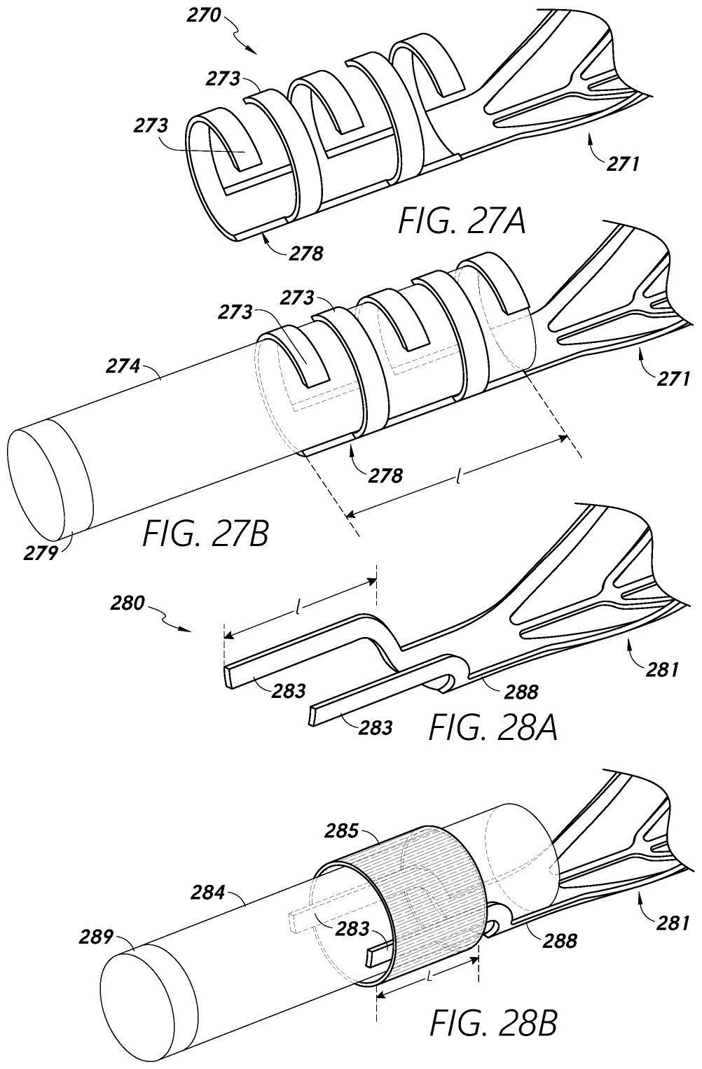

FIGS. 27A and 27B illustrate perspective views of at least a portion of a medical implant device 270 including a sensor-support arm structure 278 and one or more opposing circumferentially-wrapping fingers 273, which may be used to provide a tension and/or enclosure fit to secure a sensor device 274 therein, as shown in FIG. 27B. The fingers 273 may be axially/longitudinally offset from one another, as shown in FIG. 27A. For example, the fingers on one side of the sensor-support arm 278 can be interleaved/alternating with the fingers originating from the other/opposite side of the sensor-support arm 278 when the fingers 273 are in a wrapped/sensor-retention configuration, as shown.

The retention finger(s) 273 can radially compress against the outer surface of the sensor cylinder 274. The compression shape and force of the finger(s) 273 may be provided through shape memory setting. Although five fingers 273 are shown, it should be understood that the sensor-retention structure 270 may comprise any suitable or desirable number of retention fingers. Furthermore, although fingers are shown as originating from both longitudinal sides of the support arm/strut 278, in some embodiments, finger(s) originate from only one side of the support arm 278.

FIGS. 28A and 28B illustrate perspective views of at least a portion of a medical implant device 280 including one or more prongs 283 connected to a sensor-support arm/structure 288. In some implementations, a sensor device 284 may be disposed between, adjacent to, in proximity to, and/or otherwise in relative position with the prong(s) 283, wherein a sleeve 285 or other circumferential support feature may be placed, wrapped, and/or disposed around the sensor 284 and the prong(s) 283 to thereby secure the sensor device 284 to the prong(s) 283 and/or sensor-support arm/structure 288. In some contexts, the prongs 283 may be considered and/or described as struts. As shown, the prongs/struts 283 may project generally distally. For example, the struts/prongs 283 may project generally longitudinally/axially for a length L, as shown.

The prongs 283 sleeve 285 may comprise carbothane or any other material, whether rigid or flexible. In some implementations, the sleeve/material 285 may be formed/disposed over the sensor 284 over at least a portion of the length thereof, wherein the prongs 283 may subsequently be slid between the sleeve/material 285. The sleeve material may then be re-flowed over the sensor 284, sleeve 285, and/or prongs 283 and heat-shrunk to enhance the retention characteristics of the sleeve 285. The prongs 283 may have certain bend features to prevent the prongs 283 from sliding straight out from under the sleeve 285. The prongs 283 may have a length that protrudes beyond the sleeve 285 with respect to a length of the sensor 284 and/or prongs 283.

### Proximal Sensor-Attachment Features

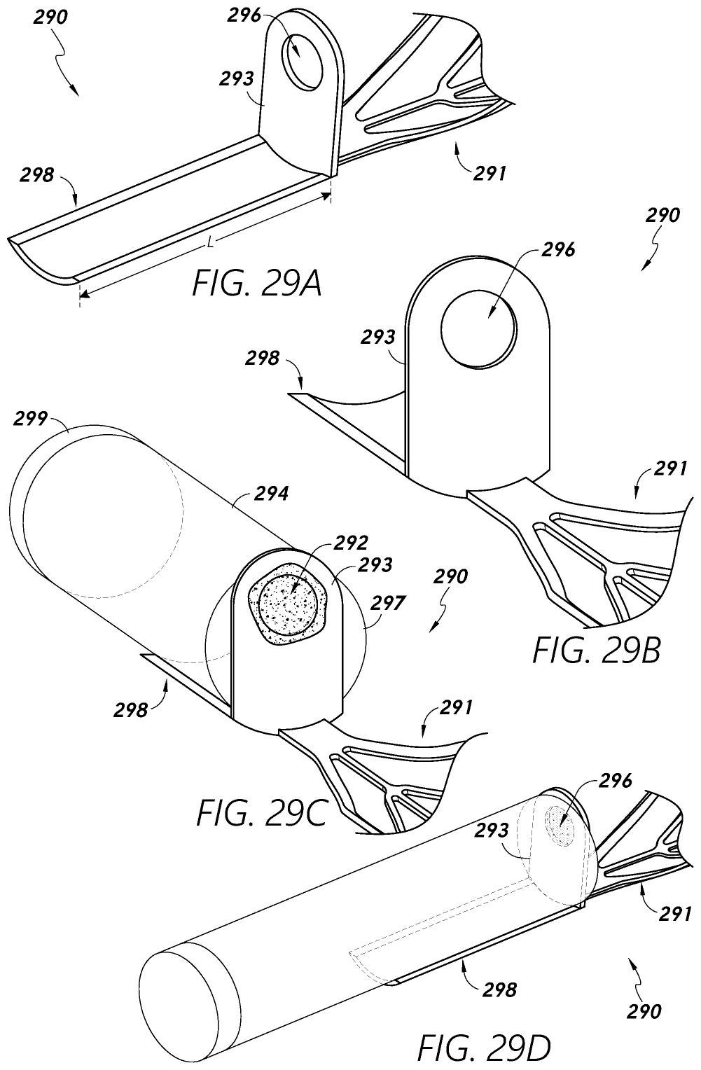

FIGS. 29A through 29D illustrate perspective views of at least a portion of a medical implant device 290 including a sensor-support structure/arm 298 including a proximal stopper feature 293. The proximal stopper feature can include an aperture, hole, or the like 296, wherein a sensor device 294 may be disposed on the sensor-support structure 298 such that a proximal end 297 thereof is positioned in proximity to the proximal stopper 293 and/or aperture 296. In some implementations, an adhesive 292 may be disposed at least partially within the aperture 296 to thereby at least partially secure the sensor device 294 and/or proximal end 297 thereof to the stopper 293, aperture 296, and/or sensor-support arm 298. The structure/form 293 may generally project radially and/or orthogonally with respect to an axis (e.g., longitudinal axis) of the sensor-support arm/strut 298. According to some embodiments, the structure/form 293 may be considered a sensor-attachment structure in that is structure 293 can be used to attach to the sensor device, thereby providing retention functionality therefore. For example, the sensor-attachment structure 293 can be configured to be attached/secured to a proximal end portion of a sensor device (e.g., cylindrical sensor device). The illustrated proximal sensor-attachment structure 293 is shown as an at least partially orthogonally projecting tab structure with an aperture therein in FIG. 29A.

The aperture 296 is shown a circular in shape but may have any suitable or desirable shape or size. In some embodiments, the aperture 296 is not closed. For example, a top portion of the aperture 296 (with respect to the illustrated orientation of FIG. 29B) is open, such that the proximal stopper feature 293 has a pronged/fork-type form.

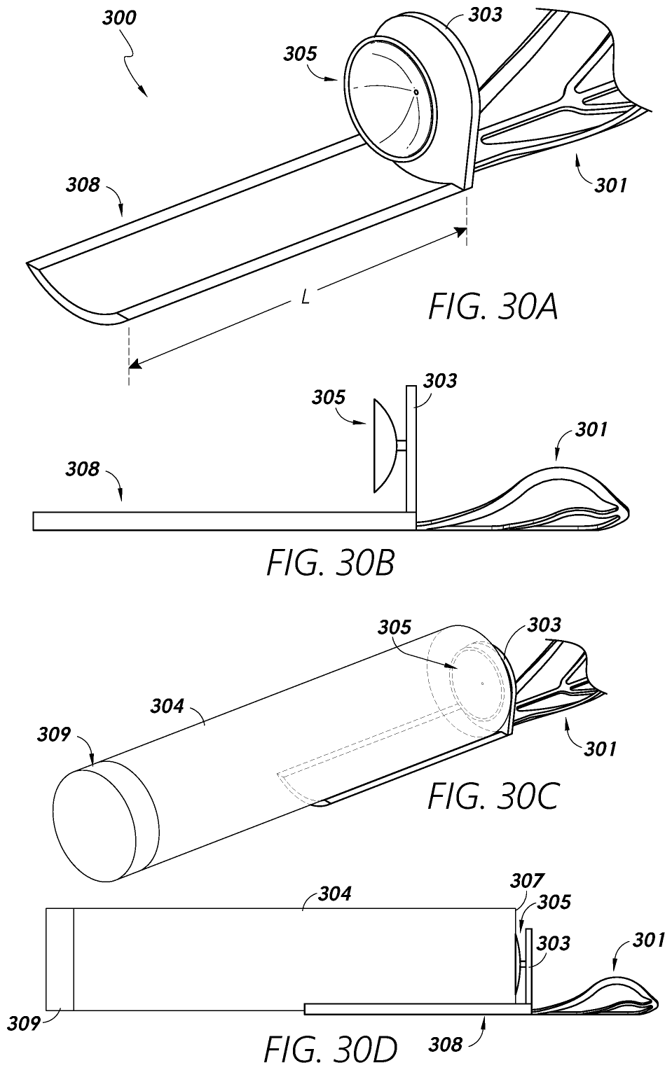

FIGS. 30A through 30D illustrate perspective views of at least a portion of medical devices including a sensor-retention structure 300 including a sensor-support structure/arm 308 and a suction-enabled proximal stopper feature 303. The proximal stopper can include one or more suction cups 305, wherein such suction cup(s) are configured such that a sensor device 304 and/or proximal surface/portion 307 thereof may be engaged with the suction cup(s) 305 to thereby suction the sensor device 304 and/or proximal end 307 thereof to the proximal stopper 303 and/or sensor-support structure/arm 308. In some embodiments, adhesive may be used to hold the suction cup(s) 305 against the proximal end of the sensor 304. Although the suction cup 305 is shown at a proximal end of the support arm 308, in some embodiments, one or more suction cups may be positioned on the support arm 308 (e.g., facing upward with respect to the illustrated orientation in FIG. 30B).

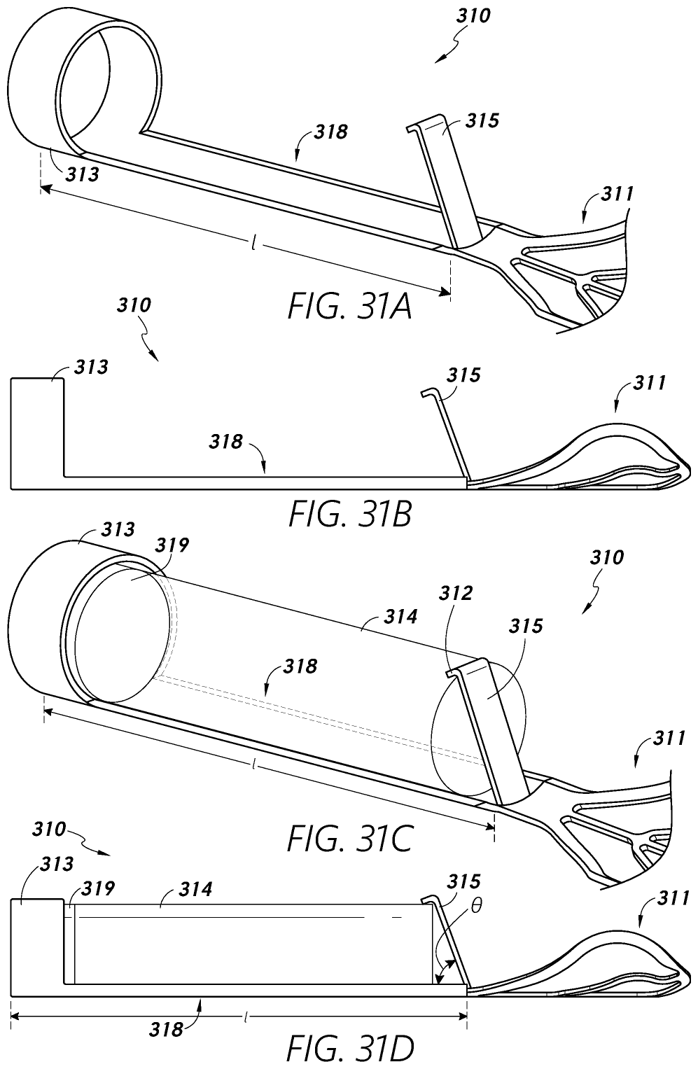

FIGS. 31A through 31D illustrate perspective views of at least a portion of a medical implant device including a sensor-retention structure 310 that includes a proximally-positioned and distally-angled stopper and/or retention arm/structure 315. The sensor-retention structure 310 may further comprise one or more circumferential sensor retention bands, such as illustrated distal retention band 313. The sensor-support structure 318 may further be positioned and/or configured to provide support for a longitudinal portion of the sensor device 314. The proximal arm structure 315 may be configured to engage with side and/or proximal end portions of the sensor device 314. For example, the arm structure 315 may attached to the proximal end of sensor device 314 using a hook feature 312 associated with a distal end of the arm 315. In some embodiments, the arm structure 315 is configured to present spring tension force against the sensor device 314.

The proximal retention arm 315 may be at least partially flexible, such that it can accommodate and/or allow for variations in size/length and/or position of sensor devices that may be disposed on the arm 318. That is, the angle θ of the proximal retention arm 315 with respect to the arm/support structure 318 may be adjustable to fit on the particular sensor disposed on the arm 318.

The proximal retention arm 315 may push against the sensor 314 both in the direction toward the arm 318 as well as in the distal direction to some degree. Therefore, it may be desirable for the distal retention band 313 to include shoulder/ridge feature(s), as described herein in connection with various other embodiments and figures, to prevent distal axial sliding of the sensor 314.

Sensor-Support Arms with Retention Characteristics

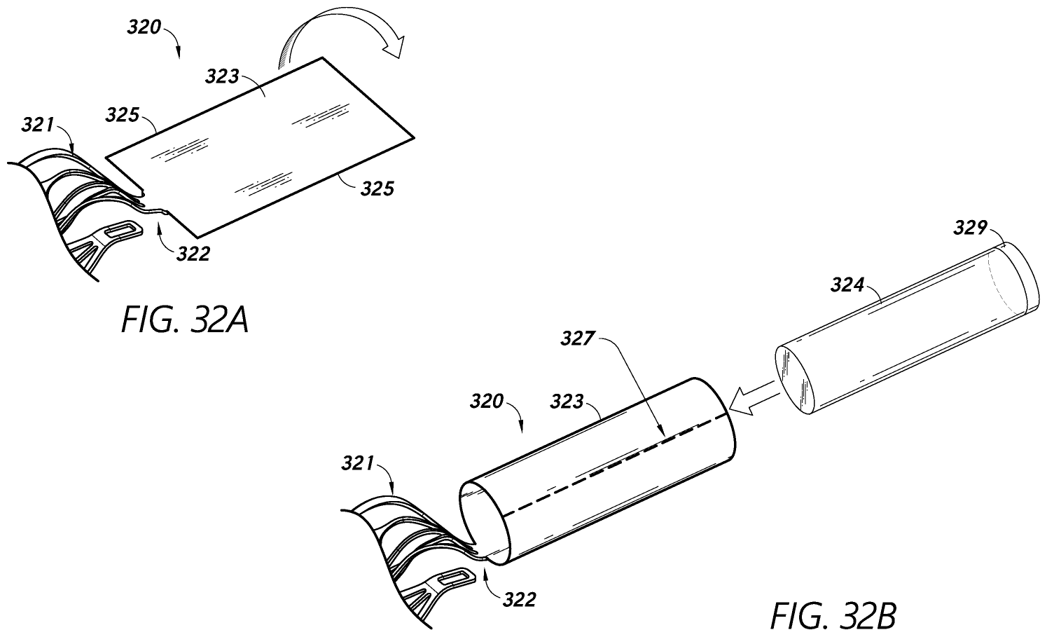

FIGS. 32A and 32B illustrate perspective views of at least a portion of a medical implant device including a sensor-retention structure 320 including a sheet or other structure 323, that may be at least partially flat and/or curved at one or more points in a manufacturing, delivery, and/or implantation process, and that is configured to be wrapped at least partially around a sensor device 324 to thereby provide a tension/pressure fit with the sensor device 324 to thereby secure the sensor device 324 to the sensor-retention structure 320 and/or associated medical implant device. Radial compressive forces imposed by the sheet 323 can effectively hold the sensor 324 in place and may prevent one or both of axial movement of the sensor 324 on/within the sheet 323 and movement of the sensor 324 away from the base 322 of the sheet arm 323. The sheet 323 may be wrapped into an at least partially cylindrical or curved configuration prior to insertion of the sensor 324 or may be wrapped around the sensor 324 after the sensor 324 has been placed thereon.

The sheet 323 can allow for some amount of expansion and therefore may be suitable for sensor devices having various diameters or other dimensions. In some implementations, the edges 325 sheet 323 are not brought into contact with one another in the wrapped configuration, but rather form a ‘C’ shape around the sensor 324 and/or into which the sensor 324 may be placed. The sheet 323 may be a laser-cut metal or plastic, bendable form. As with any of the embodiments of the present disclosure, the retention form of the sensor-retention structure 320 (e.g., the wrapped configuration of the sheet 323) may be achieved through shape memory of one or more components thereof. For example, the sheet 323 may be shape-set to the wrapped shape and may assume such shape automatically when deployed from the delivery system or freed from any type of restraints used therewith.

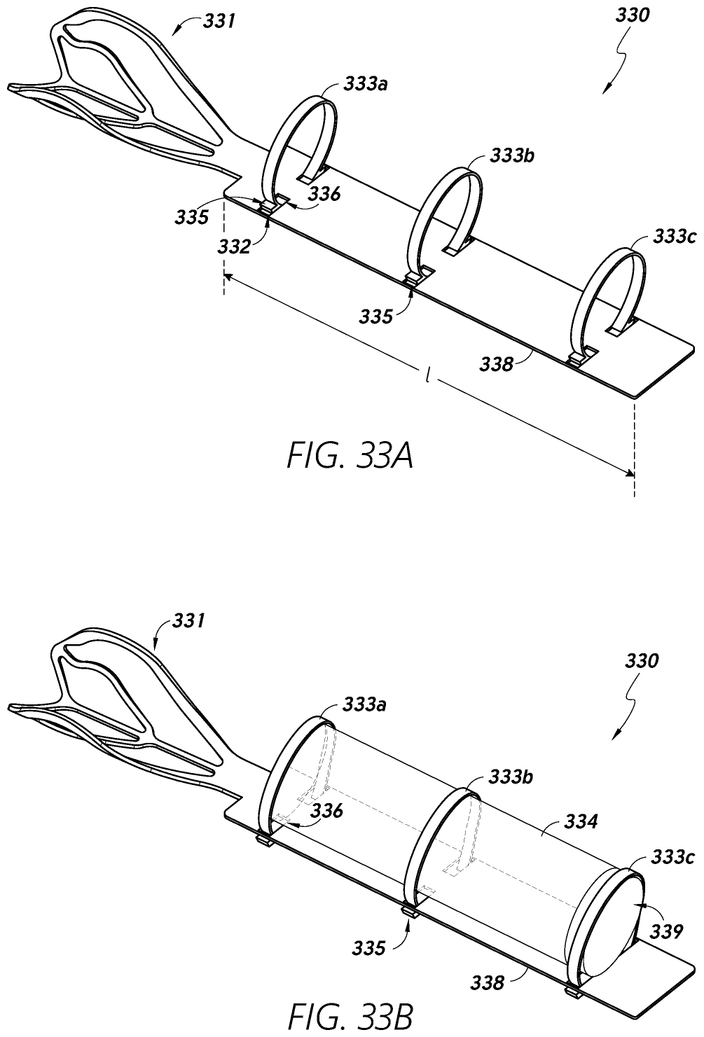

FIGS. 33A and 33B illustrate perspective views of at least a portion of the medical implant device including a sensor-retention structure 330 including a sensor-support arm structure 338 and one or more sensor-retention rings 333. In some embodiments, the sensor-support arm includes one or more apertures 336 and/or other features configured to allow for portions of the sensor-retention rings 333 to be snapped, hooked, clipped, and/or otherwise attached or secured thereto/in. For example, the sensor-retention rings 333 may comprise hook features 335, or the like, configured to be inserted at least partially into the apertures 336 or other like features of the sensor-support arm 338 and become hooked/clamped therein. For example, the hook features 335 may be configured to hook onto perimeter bands/portions 332 of the sensor-support arm 338.

The sensor-support arm/strut 338 can provide mechanical support for the sensor 334 as well as a dock to which the sensor-retention rings 333 can anchor. In some embodiments, the curved shape of the ring(s) 333 is achieved, at least in part, using shape memory setting. Furthermore, the tendency of the rings 333 to bow outward, which can advantageously provide retention force against the outside/perimeter bands/portions 332, can be from shape memory characteristics of the rings 333, which may create a tension in the rings 333 when bent into the curved configuration of FIGS. 33A and 33B. That is, the rings, when compressed/curved as shown in FIGS. 33A and 33B, may have an outward spring-type force that causes the rings to want to expand and straighten out to some degree Although three sensor-retention rings are shown in FIGS. 33A and 33B, it should be understood that the sensor-retention structure 330 may comprise any suitable or desirable number of rings and/or ring-docking features, including embodiments comprising a single sensor-retention ring.

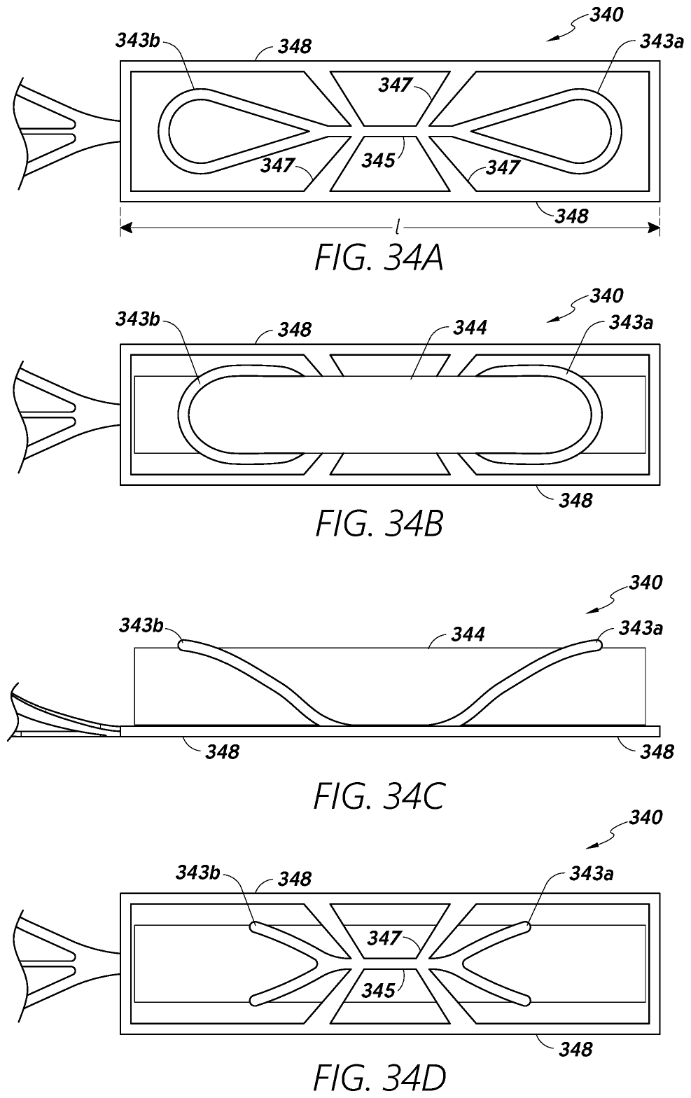

FIGS. 34A through 34D illustrate top, bottom, and side views of at least a portion of a medical implant device including a sensor-retention structure 340. The sensor-retention structure 340 advantageously includes a sensor-support arm structure 348 having associated therewith one or more retention hoops 343. The sensor-retention hoops 343 may advantageously be configured to be projected away from the sensor-support structure 348 to allow for insertion at least partially therein of one or more portions of a sensor device 344.

The retention hoops/eyelets 343 can exert downward force on the sensor 344 towards the sensor-support arm/structure 348 to hold the sensor 344 against the support arm/structure 348 and/or prevent axial sliding of the sensor 344. The downward forces of the hoops 343 on the distal and proximal ends of the sensor 344 ideally is not sufficient to damage/break the sensor 344. In some embodiments, as with any of the other embodiments disclosed herein, one or more portions (e.g., portion(s) of the hoops 343) of the sensor-retention structure 340 may be dipped or coated in a relatively tacky polymer/rubber, such as carbothane or the like. Such coating may increase the friction between the sensor-retention structure 340 and the sensor 344.

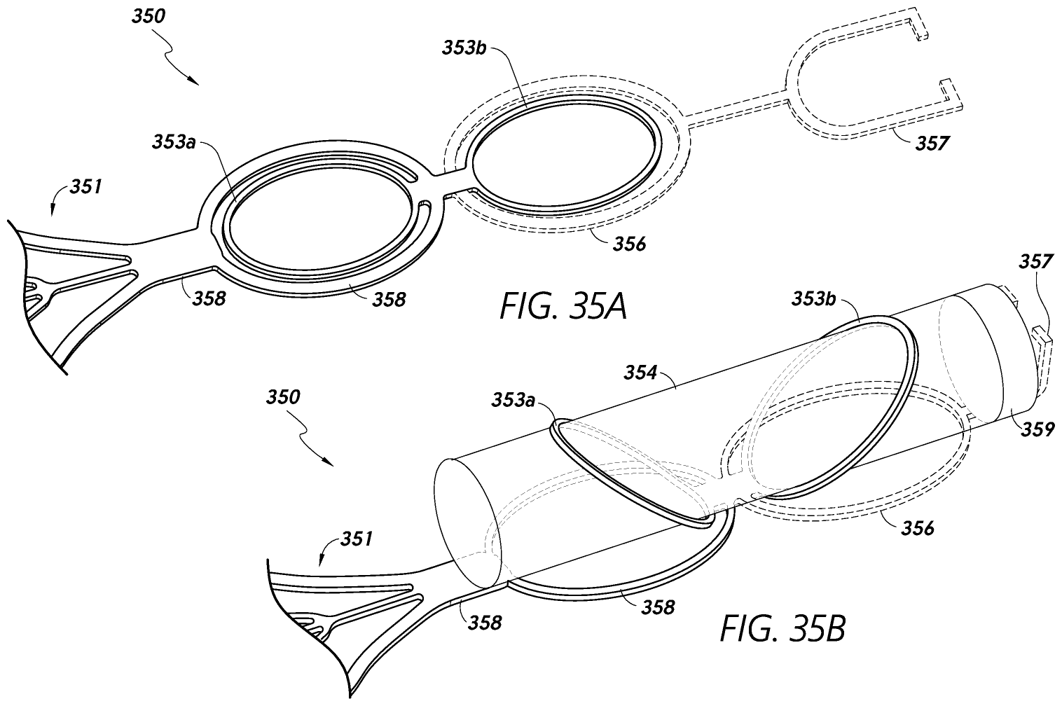

FIGS. 35A and 35B illustrate perspective views of at least a portion of a medical implant device including a sensor-retention structure 350. The sensor-retention structure 350 advantageously includes a sensor-support arm structure 358 having associated therewith one or more retention hoops 353. The sensor-retention hoops 353 may advantageously be configured to be projected away from the sensor-support structure 358 to allow for insertion at least partially therein of one or more portions of a sensor device 354. The sensor-retention structure 350 can further comprise, in some embodiments, a distal stopper 357, which may be bent or otherwise configured to prevent distal movement of the sensor 354 past the distal stopper 357.

In some embodiments, shape memory characteristics of the sensor-retention hoop(s) 353 may cause the hoop(s) to have a tendency to push downward toward the sensor-support structure 358. Such tensile forces can press the hoops 353 against the sensor 354 to help hold the sensor 354 and/or create friction between the hoops 353 and the sensor 354 to prevent the sensor 354 from sliding axially; radial movement of the sensor 354 may be generally restrained by the wrapping of the hoops around the circumference of the sensor 354.

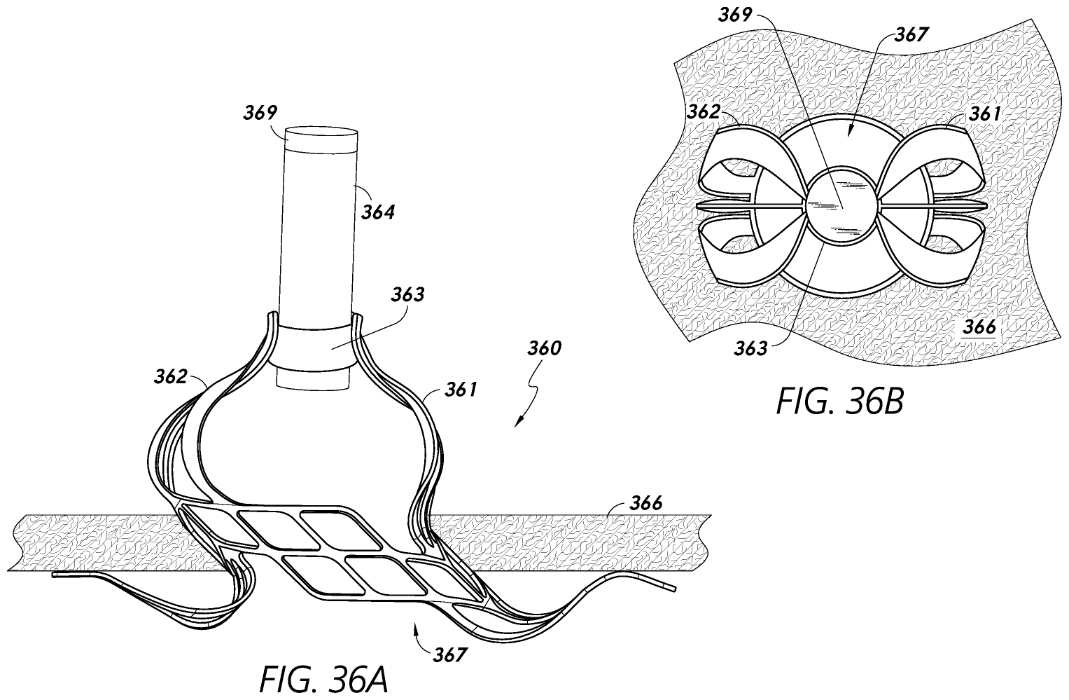

FIGS. 36A and 36B illustrate side and axial views, respectively, of a medical implant device 360 comprising certain sensor-retention features. For example, the medical implant device 360 may be a shunt-type device, or any other type of a medical implant device including one or more arms 361, 362. The medical implant device 360 includes a plurality of arms 361, 362 configured to be oriented to project in an axial direction with respect to a shunt barrel 367 of the medical implant device 360. The arms 361, 362 may be brought together, wherein a sensor device 364 may be secured at least in part by the arms 361, 362 and/or one or more bands or other sensor-retention features 363 disposed or attached at least partially therebetween. Therefore, the arms 361, 362 and the sensor-retention band 363 may provide a sensor holder structure, as shown.

FIGS. 36A and 36B show the implant arms 361, 362 projecting away from the tissue wall 366 to hold the sensor 364. However, in some embodiments, additional arms may be used to secure the implant device 360 against the tissue wall 366 in addition to any arm structures that extent away from the wall 366. That is, the implant device may include sensor-holding arms/structures and implant-securing arms-structures on the side of the implant device 360 on which the sensor 364 is held.

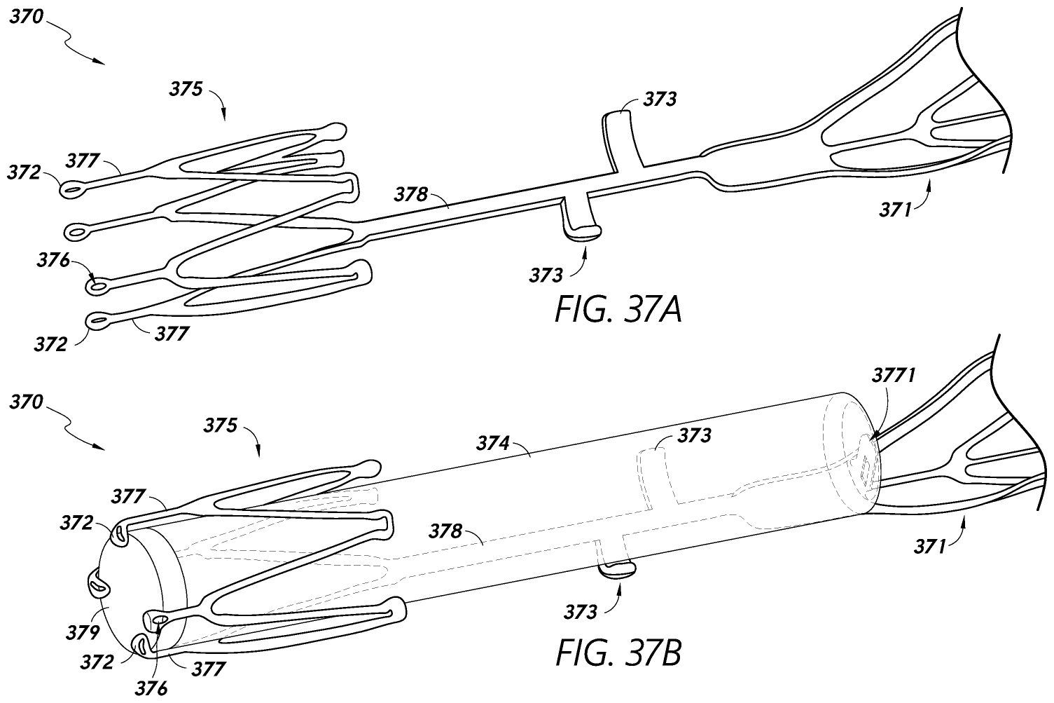

FIGS. 37A and 37B illustrate perspective views of at least a portion of a medical implant device including a sensor-retention structure 370. The sensor-retention structure 370 can advantageously comprise a distal sensor-retention cage structure 375, which may be attached to or otherwise associated with a sensor-support arm 378. The sensor-support arm 378 may further include or have associated therewith one or more opposing fingers 373, which may be configurable to provide retention and/support functionality for a sensor device 374. The sensor-support structure 370 may further comprise one or more distal stopper tabs 372, which may be attached to and/or otherwise associated with the distal retention cage 375, as shown.

The cage structure 375 can provide an adjustable sensor-retention structure. For example, the distal stopper tabs 372 may be bent inward by bending the struts 377 coupled thereto at a desired point on the struts 377 that results in a desired length of the cage to fit the particular sensor device. The distal tabs 372 and/or struts 377 can be shape-set according to shape memory characteristics thereof to bend inward to the degree desired to constrain the sensor 374. The distal tabs 372 may have apertures 376 or other features therein. Such features may have sutures or other features engaged therein to further secure the tabs 372 in place and/or provide axial retention for the sensor 374.

The sensor-retention structure 375 may include a proximal stopper feature 3771, such as a tab form or the like. The tab 3771 may be foldable and/or inclined to fold due to shape memory characteristics thereof to thereby project away from the arm 378 and provide a surface against which the proximal end of the sensor 374 can rest or be pressed, such that axial movement of the sensor in the proximal direction is constrained. It should be understood that any of the embodiments disclosed herein may have proximal and/or distal stopper tabs, which may be shape-set to assume a position projecting into the axial path of a retained sensor to constrain axial movement thereof.

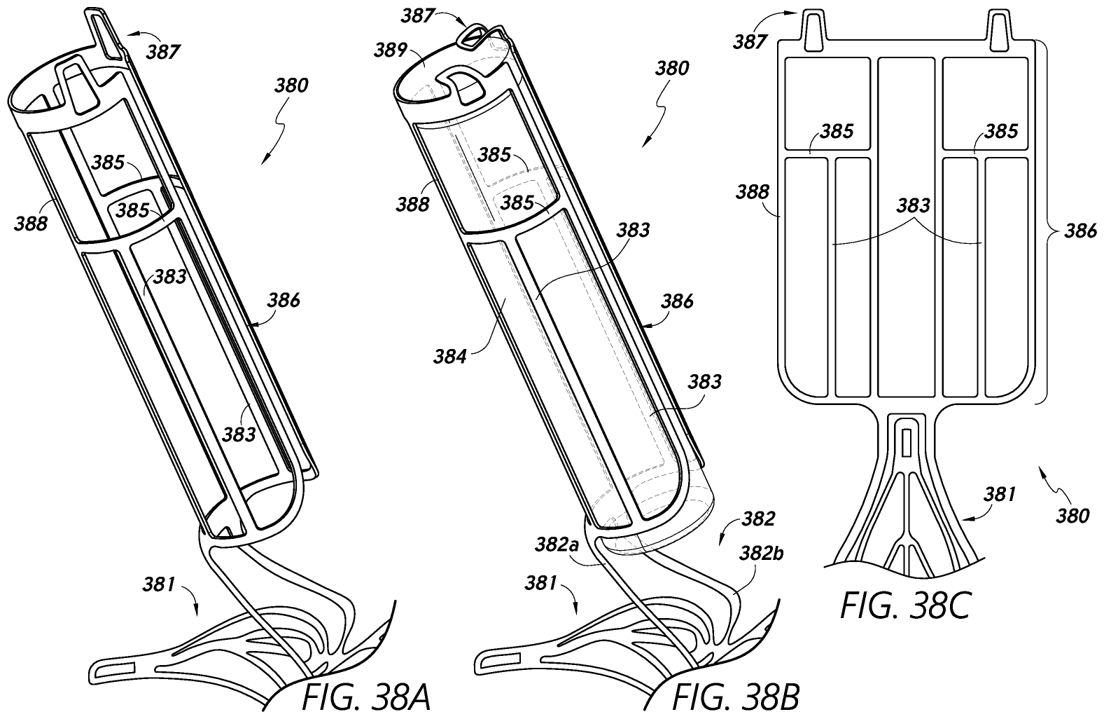

FIGS. 38A and 38B illustrate perspective views of at least a portion of a medical implant device including a sensor-retention structure 380. FIG. 38C shows a flattened-out configuration of the sensor-retention structure 380 in accordance with one or more embodiments. The sensor-retention structure 380 includes a cage structure 386 including one or more longitudinal struts 383, 388 and/or circumferential struts 385, as shown. The sensor-retention structure 380 can further comprise one or more distal stopper tabs 387, which may be bendable, foldable, and/or otherwise configurable to at least partially prevent or reduce distal movement of the sensor 384 when the sensor 384 is at least partially enclosed within the cage structure 386 of the sensor-retention structure 380.

In some embodiments, the sensor-retention structure 380 is integrated with or coupled to an auxiliary sensor-support arm 382 that is associated with the sensor-retention structure 380 of an implant device and is provided in addition to the tissue-engagement implant-support arm 381. It should be understood that any of the embodiments of sensor-retention arms/structures disclosed herein may be coupled with, attached to, integrated with, or otherwise associated with a dedicated/auxiliary sensor-support arm, rather than with an arm that is used for tissue engagement for the purpose of implant device stabilization. Such dedicated and/or auxiliary sensor-support arms may have a jaw-type configuration like that shown in FIGS. 38A and 38B. For example, in some cases, an auxiliary sensor-support arm may include first 382a and second 382b arms that connect at or near a base of a tissue-engagement arm 381 and are integrated with a cage-like or other sensor-retention structure as shown in FIGS. 38A and 38B. With respect to any embodiment of an implant device including an auxiliary sensor-support arm (e.g., like that shown in FIGS. 38A and 38B in one or more respects), such auxiliary sensor-support arm may, in a deployed configuration, project away from the tissue wall in which the implant device is implanted to a greater degree/angle than an associated tissue-engagement arm if the implant device that is disposed on the same side/area of the implant device as the auxiliary arm. One having ordinary skill in the art should understand that the arm components 382, or similar auxiliary arm struts/components, can be coupled to the various other embodiments of the present disclosure.

Sensor-Support Arms Associated with Shunt Structure

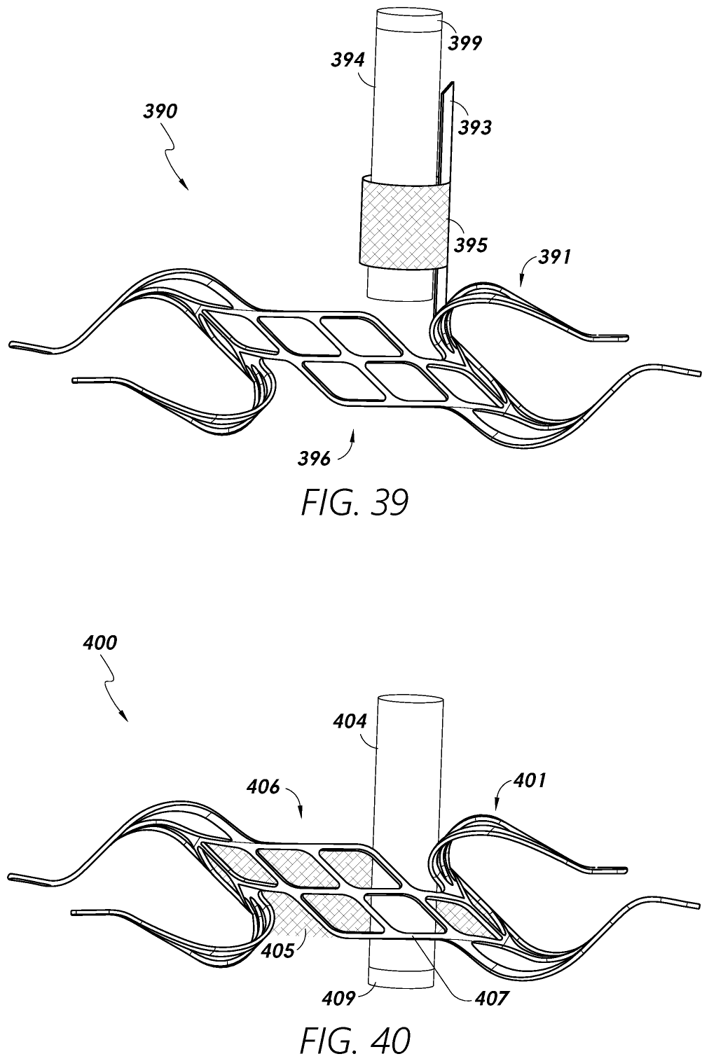

FIG. 39 illustrates a side view of a medical implant device 390 including an axial sensor-support arm 393 configured to hold a sensor device 394 at least partially within an axial path of a shunt barrel 396 associated with medical implant device 390. In some embodiments, a band or other features 395 may at least partially secure the sensor device 394 to the sensor-support arm 393.

For illustrative purposes, the following text describes the medical implant device 390 of FIG. 39 as a shunt structure. However, it should be understood that the medical implant device 390 may be any suitable or desirable type of a medical implant device. In the illustrated embodiment of FIG. 39, the sensor element 399 is attached to or integrated with the arm member 393, which extends generally along a longitudinal dimension or axis of the shunt structure 390. However, in certain embodiments, the arm 393 extends along a dimension parallel and/or angled with respect to the flow path, but on an outside of the barrel. In the embodiment illustrated in FIG. 39, the sensor 394 is attached to, or otherwise integrated with, the flow path barrel or conduit portion of the shunt structure 396, such that the sensor 394 is disposed within or near the flow path channel associated with the barrel/conduit 396.

FIG. 40 shows a side view of another embodiment of a shunt-type medical implant device 400 having a sensor device 404 secured at least in part thereto. In particular, the sensor device 404 can be disposed at least partially within a shunt barrel 406 of the implant device 400. For example, the sensor device 404 may be secured to the barrel structure in some manner. In some embodiments, an inner lining (e.g., memory metal and/or polymer mesh or sleeve) may be disposed at least partially within the barrel 406, such that the sensor 404 is secured at least in part through friction and/or pressure force between the mesh/sleeve 45 and the barrel structure. The mesh/sleeve 405 can be secured/attached in some manner to the barrel structure 406, such as through the use of sutures, hooks, adhesive, and/or other attachment means.

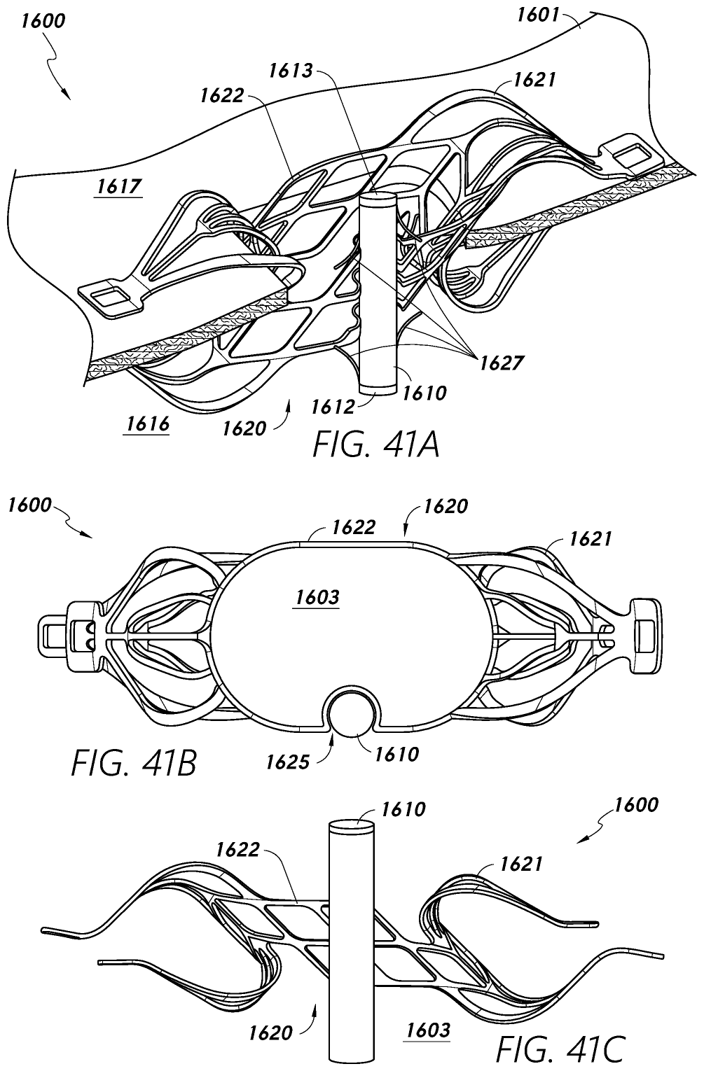

FIGS. 41A through 41C illustrate another embodiment of a shunt-type medical implant device 1600 having a sensor device 1610 secured at least in part thereto. FIG. 41A is a perspective view of the sensor implant device 1600. In the embodiment illustrated in FIG. 41A, the sensor 1610 has an elongate cylindrical shape, as with certain other embodiments disclosed herein. However, it should be understood that sensors in accordance with embodiments of the present disclosure may have any form, shape, configuration and/or orientation.

In some embodiments, the sensor 1610 includes a first sensor element 1612 at a first end of the sensor 1610 that is disposed on a first side 1616 of a tissue wall 1601 and the shunt structure 1620 when implanted in a patient, such as in a wall separating the coronary sinus from the left atrium. For example, the sensor 1612 may be positioned to be exposed within the left atrium, which may be represented by the side or region 1616 in the illustrated diagram. The sensor 1610 may further comprise a second sensor 1613 disposed on an opposite side of the sensor 1610. For example, the sensor 1613 may be configured and positioned to be exposed in a chamber or area associated with an opposite side of the tissue wall 1601 and/or shunt structure 1620, such as within the coronary sinus. With respect to interatrial shunting, the sensor elements 1612, 1613 may be disposed or positioned in respective atria, wherein one sensor element provides pressure readings associated with the left atrium and the other provides pressure readings associated with the right atrium, as described in detail above. Using two sensor elements as shown in FIG. 41A may allow for measurement of differential pressure drop across the shunt structure 1620.

With the sensor 1610 disposed or attached in or near the flow path channel of the shunt structure 1620, the sensor 1610 may be configured to provide sensor readings that can be used to indirectly measure flow across or through the shunt structures 1620 based at least in part on fluid momentum associated with fluid contacting the sensor element(s). Furthermore, the sensor(s) may generate readings relating to velocity of flow through the shunt, wherein such readings may be used to determine or indicate undesirable occlusion or closing-off of the shunt flow path. In certain embodiments, pressure waveforms generated using the sensor(s) may be used to generate and/or maintain a waveform profile relating to the pressure readings. Changes in the pressure reading profile may indicate health complications and may therefore be used to trigger alerts or notifications, which may be relied upon to alter drug or other therapies. In some embodiments, the pressure readings from the sensor(s) are analyzed to determine average, diastolic, and/or systolic pressure data points.

In the embodiments of FIGS. 41A through 16C, the sensor 1610 is mounted or attached at or near the orifice or channel of the shunt structure 1620, rather than to one or more arm members, as described above. The sensor 1610 may be built into, or otherwise integrated with, the shunt structure 1620, or may be otherwise attached or associated therewith. Although FIGS. 41A through 16C illustrate the sensor 1610 attached or integrated with the barrel/conduit portion 1622 of the shunt structure 1620 on an outside surface thereof, it should be understood that in some embodiments the sensor 1610 may be disposed, attached, or otherwise integrated with the barrel/conduit portion 1622 on an inside surface thereof. With respect to the illustrated embodiment, the sensor 1610 may fit within a pocket/receptacle feature 1625 of the barrel/conduit 1622. In some embodiments, the sensor 1610 comprises or is associated with one or more projections 1627 configured or designed to hold or secure the sensor 1610 to the shunt structure 1620.

FIG. 41B shows a front view of the sensor implant device 1600 in accordance with one or more embodiments. FIG. 41B shows the sensor 1610 nested in a pocket/receptacle feature 1625 associated with the barrel portion 1622 of the shunt structure 1620. FIG. 41C shows a side view of the sensor implant device 1600, showing the sensor 1610 disposed in the flow path 1603 of the shunt structure. In some embodiments, the sensor 1610 is sutured to the shunt structure. In some embodiments, the sensor 1610 is held within a sock or pouch that is sutured or otherwise attached to the shunt structure 1620.

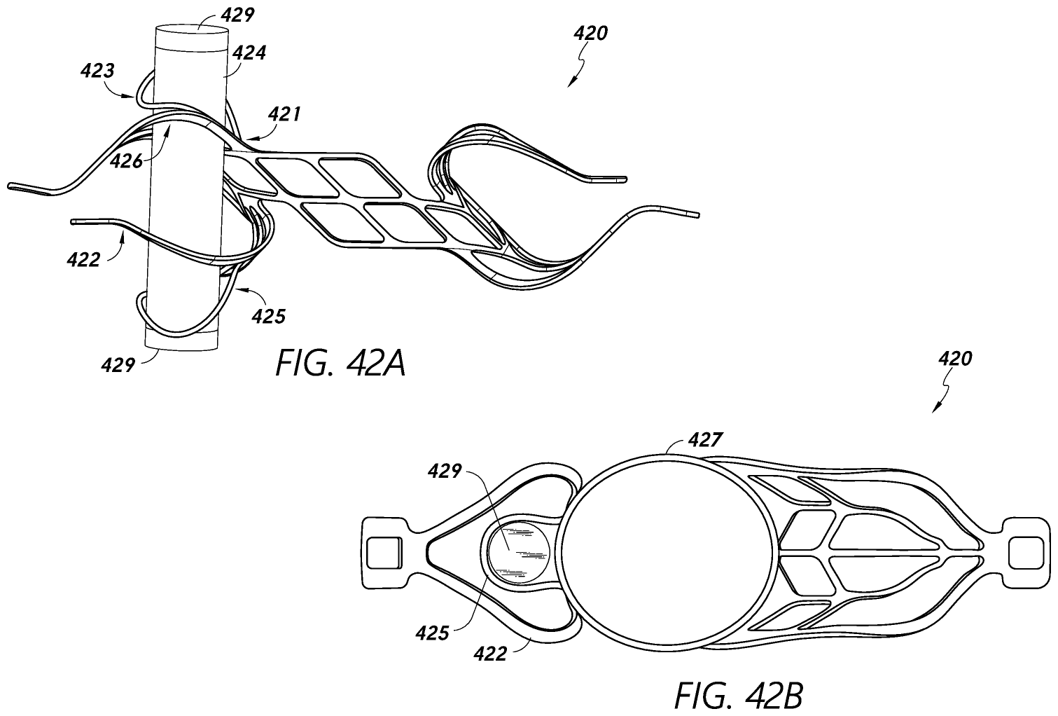

FIGS. 42A and 42B illustrate side and axial views, respectively, of another embodiment of a shunt-type medical implant device 420 including certain sensor retention features. The medical implant device 420 may be configured to at least partially secure a sensor device 424 in a position at least partially outside of a barrel structure 427 of the implant device 420 and/or generally parallel to a flow axis/path associated with the shunt barrel 427. The sensor-retention features of the medical implant device 420 can include one or more sensor-retention hoops, rings, arms/fingers or the like 423, 425, as shown, which may be configurable to secure the sensor 425 using production and/or pressure forces. Although the features 423, 425 are shown as projecting away from the respective arms 421, 422, in some embodiments, the retention features 423, 425 are substantially in-line with the respective arms 421, 422, or deflect inward with respect to the respective arms 421, 422. Furthermore, although two retention features 421, 425, in some embodiments, only a single sensor-retention hoop/feature is included.

### Additional Sensor-Retention Structures

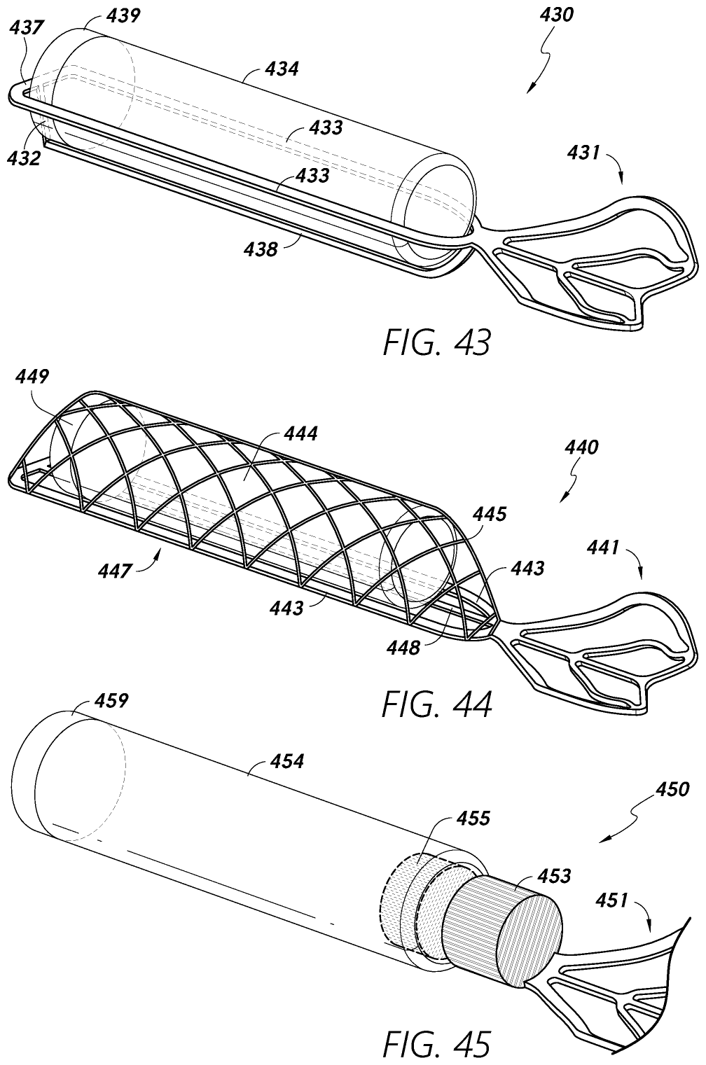

FIG. 43 illustrates a perspective view of at least a portion of a medical implant device including a sensor-retention structure 430. The sensor-retention structure 430 includes a plurality of sensor-support struts, including one or more side struts 433 and one or more radially-projecting posterior support struts 438. The sensor-support struts 433, 438 may generally form a trough-type support form in which a sensor device 434 may be at least partially disposed and/or secured.

The contact between the sensor-retention structure 430 and the sensor membrane (e.g., distal face of sensor element 439) may advantageously be minimized to reduce interference with the function of the sensor element 439. For example, the strut 432 coupling the posterior support strut 438 to the distal crossbar strut 437 is angled axially outward with respect to the sensor orientation shown to prevent the majority of the length of the connecting strut 432 from contacting the sensor element 439. In some embodiments, one or both of the connecting strut 432 and the crossbar strut 437 is omitted in order to reduce contact with the sensor element 439. For example, the side 433 and posterior 438 struts can be prongs that are not coupled at the distal ends thereof. In such embodiments, the struts 433, 438 may have connecting struts therebetween at one or more positions along the length thereof to provide mechanical stability.

FIG. 44 illustrates a perspective view of at least a portion of a medical implant device including a sensor-retention structure 440. The sensor-retention structure 440 includes a sensor-support arm 447 including one or more sensor-support struts 443, 448. In some embodiments, the sensor-support arm 447 includes one or more outer support struts 443, as well as a central backbone support strut 448, as shown. A braiding, netting, mesh, cloth, film, or the like may be draped or otherwise disposed over a sensor device 444 to thereby secure the sensor 444 to the sensor-support arm 447.

Although FIG. 44 shows the sensor 444 sitting on the central support strut 448, which is shown as lying in a common plane with the outer support struts, it should be understood that in some embodiments, the central support strut 448 sits at least partially below the plane of the outer struts 443. For example, the central strut 448 may provide a trough-like support similar to the support strut 438 shown in FIG. 43. Such a configuration may provide additional support for the sensor 444 with respect to the sides thereof and may further allow the distal and proximal ends of the support structure to axially restrain the sensor 444.

FIG. 45 illustrates a perspective view of at least a portion of a medical implant device including a sensor-retention structure 450, wherein the sensor-retention structure 450 comprises one or more magnets 453, which may be associated with an arm 451 of the medical implant device. The one or more magnets 453 may be configured to function as sensor-retention magnet(s), wherein a corresponding magnetic element 455 associated with a sensor device 454 may be attracted to the one or more magnets 453, thereby at least partially securing and/or positioning the sensor device 454 when the magnet(s) of the structure 450 and the corresponding magnet(s) of the sensor 454 are disposed in proximity to one another, as shown.

In some embodiments, adhesive or other attachment means may be applied to one or more portions of the sensor-support structure 450 and/or sensor 454 to supplement the fixation of the sensor 454 to the sensor-support structure 450. The sensor magnet 455 may be internal to the cylinder (e.g., glass cylinder) of the sensor 454, or may be disposed on outside of the cylinder at a proximal end thereof. Although the magnetic coupling is shown as supporting/retaining the sensor 454 at the proximal end thereof, magnets may be included that couple the sensor 454 to the sensor-support structure 450 along a length/side of the sensor cylinder. The magnet 453 may be coated with a biocompatible coating/material.

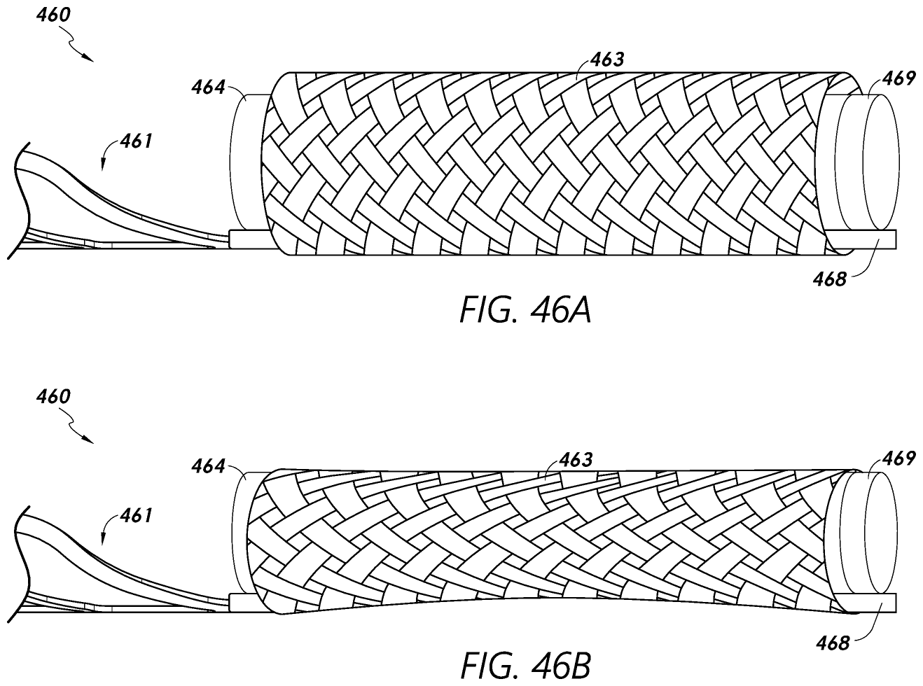

FIGS. 46A and 46B illustrate side views of a sensor-retention structure 460 in relatively shortened and lengthened configurations, respectively. The sensor-retention structure 460 may be a component or portion of a medical implant device. The sensor retention structure 460 may comprise a cylindrical, helically-wound braid, mesh, or the like. For example, the braid may advantageously be a bi-axial braid, wherein longitudinal/axial lengthening of the braid results in an at least partial narrowing of a diameter of the braid cylinder at one or more portions of the braid cylinder. In some implementations, a sensor device 464 may be placed with the cylindrical braid 463 in a relatively short/shortened configuration thereof, wherein elongation of the braid 463 results in a narrowing and/or tightening of the braid 463 around at least a portion of the sensor device 464, thereby at least partially securing the sensor device 464 in and/or to the braid 463. In some implementations, the sensor-retention structure 460 may be in the elongated/tightened configuration of FIG. 46B prior to insertion of the sensor 464. In such cases, insertion of the sensor 464 into the structure 463 can result in a friction fit between the structure 463 and the sensor 464

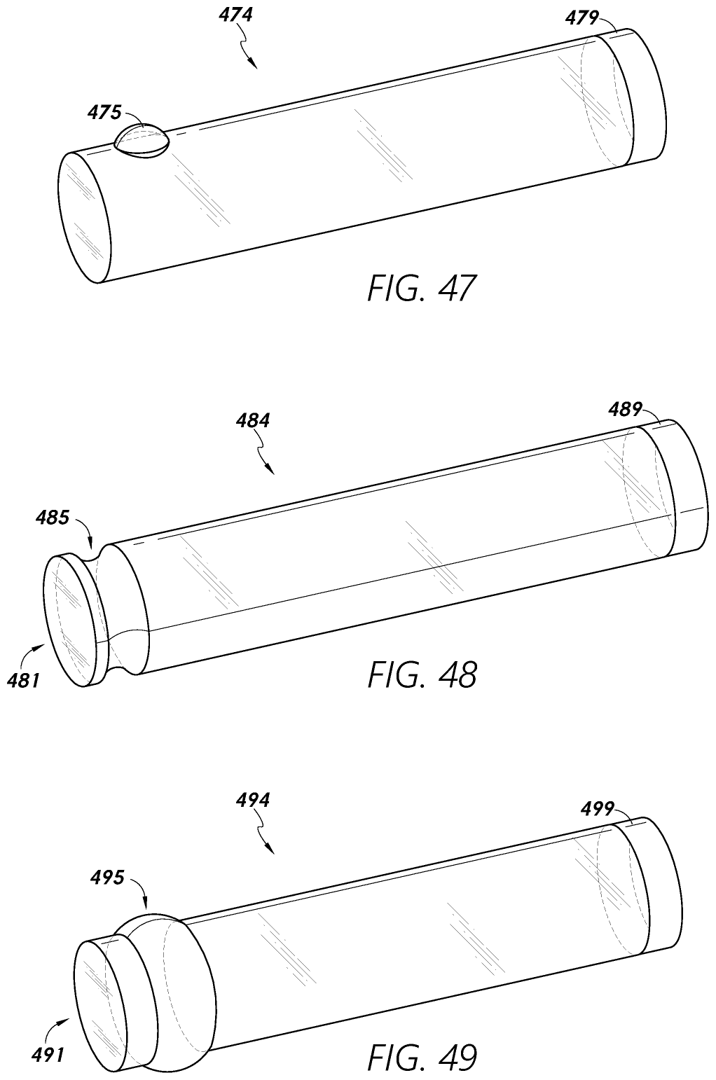

FIGS. 47 through 49 illustrate separate embodiments of sensor structures including respective sensor-retention features in accordance with one or more aspects of the present disclosure. With respect to FIG. 47, the sensor structure 474 may comprise one or more radial projections 475, which may be configured to be engaged by one or more corresponding features or components of a medical implant device to thereby at least partially secure the sensor structure 474 thereto. The projection(s) 475 can have a dot-type shape, as shown, and may have substantially rounded and/or straight edges/surfaces. The corresponding engagement features of a sensor-retention structure that engage with the projection(s) 475 can comprise a concavity that corresponds to the shape of the projection(s) 475 such that the projection(s) 475 fit relatively tightly therein to prevent movement of the sensor 474 relative to the sensor-retention structure when the projection 475 is engaged/nested in the corresponding recess/concave feature of the sensor-retention structure. Although shown as a projection, the feature 475 may be a recess/indentation in some embodiments, wherein the sensor-retention structure includes projection(s) configured to fit therein for engagement/retention.

With respect to FIG. 48, the sensor structure 484 includes a recess/trench 485, such as a circumferential recess around at least a portion of a cylindrical housing or body of the structure 484. The recess 485 may be configured to be engaged by one or more corresponding features or components of a medical implant device to thereby at least partially secure the sensor structure 44 thereto. The recess 485 may have a neck-type shape, as shown, or may have any other shape. The recess 485 can span an entire circumferential section of the sensor 484 or may cover only a portion of a circumferential section of the sensor 484. The corresponding engagement feature(s) of a sensor-retention structure that engage with the trench 485 can comprise an inwardly-projecting ring that corresponds to the shape of the trench 485 such that the ring fits relatively tightly in the trench 485 to prevent movement of the sensor 484 relative to the sensor-retention structure when the trench 485 is engaged with the corresponding ring/projection feature(s) of the sensor-retention structure. In some embodiments, the corresponding engagement feature(s) of the sensor-retention structure are sized and/or configured to snap into the trench 485 when forced over the proximal end 481 of the sensor 484 and into the trench 485.

With respect to FIG. 49, the sensor structure 494 includes an at least partially circumferential projection 495 integrated with and/or associated with or attached to a circumferential portion of a cylindrical housing or body of the structure 494. The projection 495 may be configured to be engaged by one or more corresponding features or components of a medical implant device to thereby at least partially secure the sensor structure 494 thereto.

The projection 495 may have a torus-type shape, as shown, or may have any other shape. The projection 495 can span an entire circumferential section of the sensor 494 or may cover only a portion of a circumferential section of the sensor 494. The corresponding engagement feature(s) of a sensor-retention structure that engage with the projection 495 can comprise a trench/recess that corresponds to the shape of the projection 495 such that the projection 495 fits relatively tightly in the trench/recess to prevent movement of the sensor 494 relative to the sensor-retention structure when the projection 495 is engaged with the corresponding trench/recess feature(s) of the sensor-retention structure. In some embodiments, the corresponding engagement feature(s) of the sensor-retention structure are sized and/or configured to snap onto the projection 495 when forced over the proximal end 491 of the sensor 494 and into the projection 495.

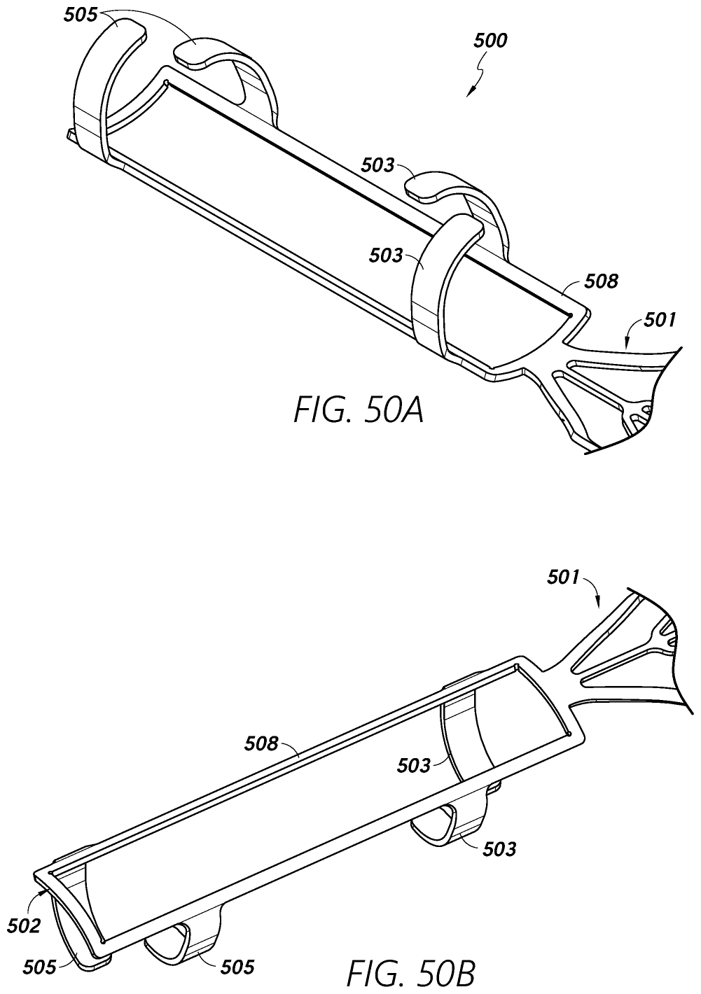

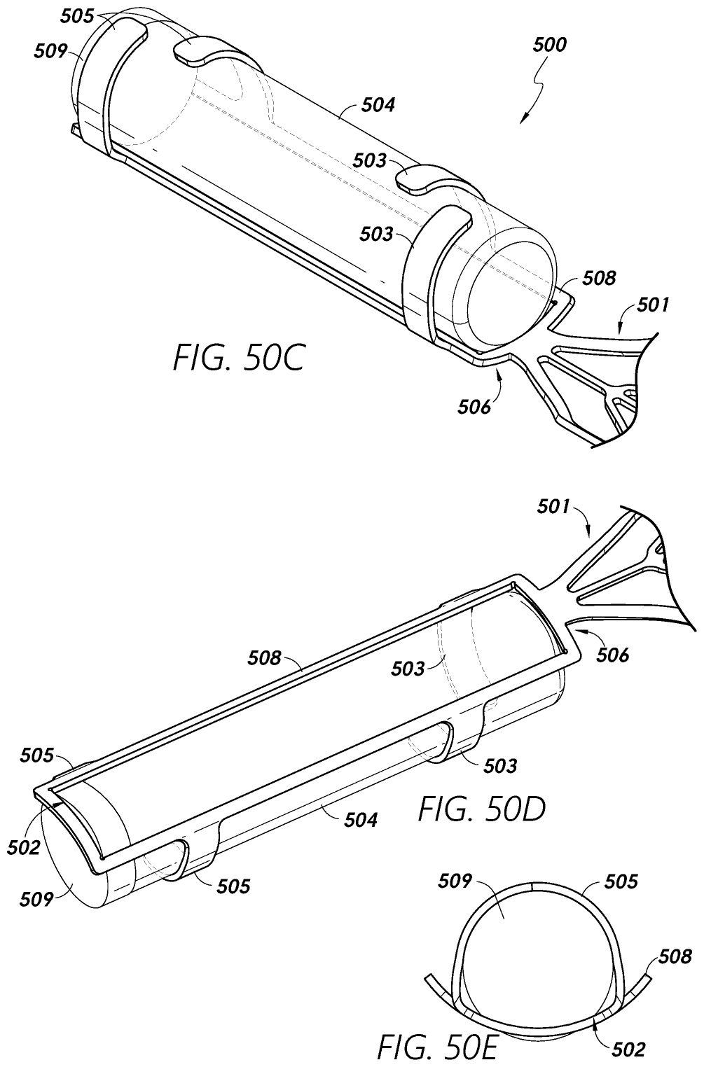

FIGS. 50A through 50E illustrate at least a portion of a medical implant device including a sensor-retention structure 500. The sensor-retention structure 500 includes a plurality of longitudinal sensor-support struts 508, which may be generally longitudinally arranged in parallel on opposite sides of the sensor-retention structure 500. The sensor-support struts 508 may generally form a trough-type support form in which a sensor device 504 may be at least partially disposed and/or secured.

The curvature of the sensor-retention structure 500 may be designed to fit and/or correspond to the radius of curvature of the outer surface of the sensor cylinder 504 to allow the sensor 504 to seat effectively thereon. Such curvature of the struts 508 may allow the sensor 504 to sit on the struts 508 in a manner that at least a portion of the sensor 504 falls below the distal 502 and/or proximal 506 cross-struts/forms, which may have a curvature that is less than (i.e., flatter) that of the outer struts 508. The relative flatness of the struts/forms 502, 506 may be designed to provide the axial retention described. In some embodiments, the curvature of the distal retention strut 502 is such that only an outer edge/portion of the sensor element 509 is contacted and/or covered by the strut 502 to avoid corrupting sensor readings and/or causing damage to the sensor element 509.

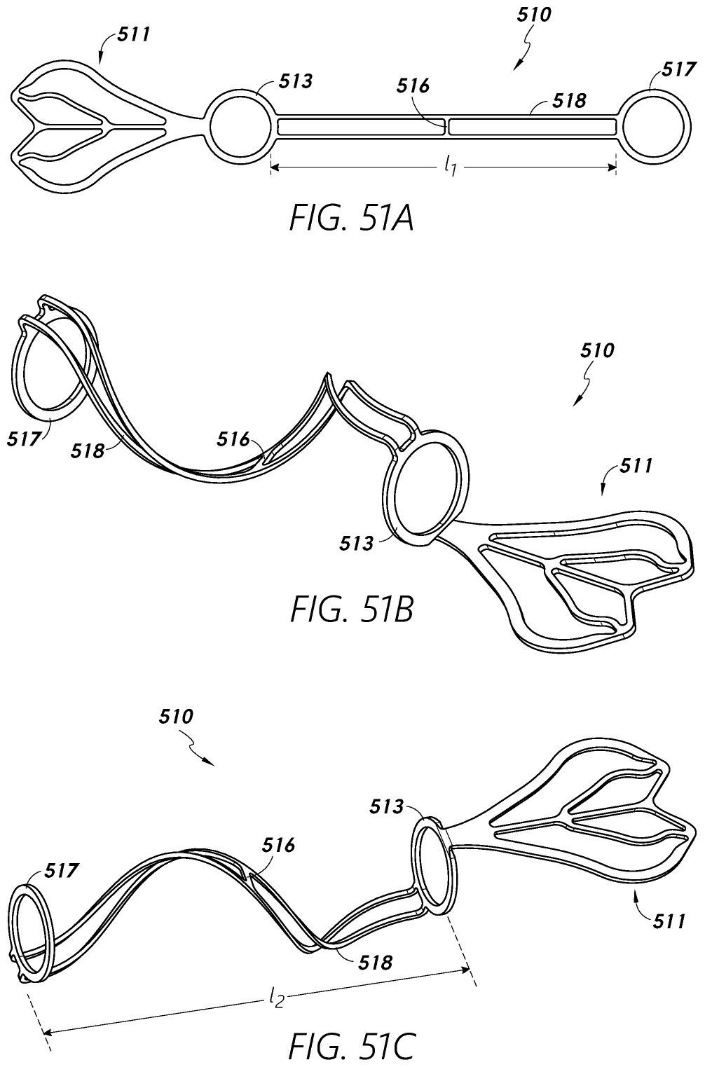

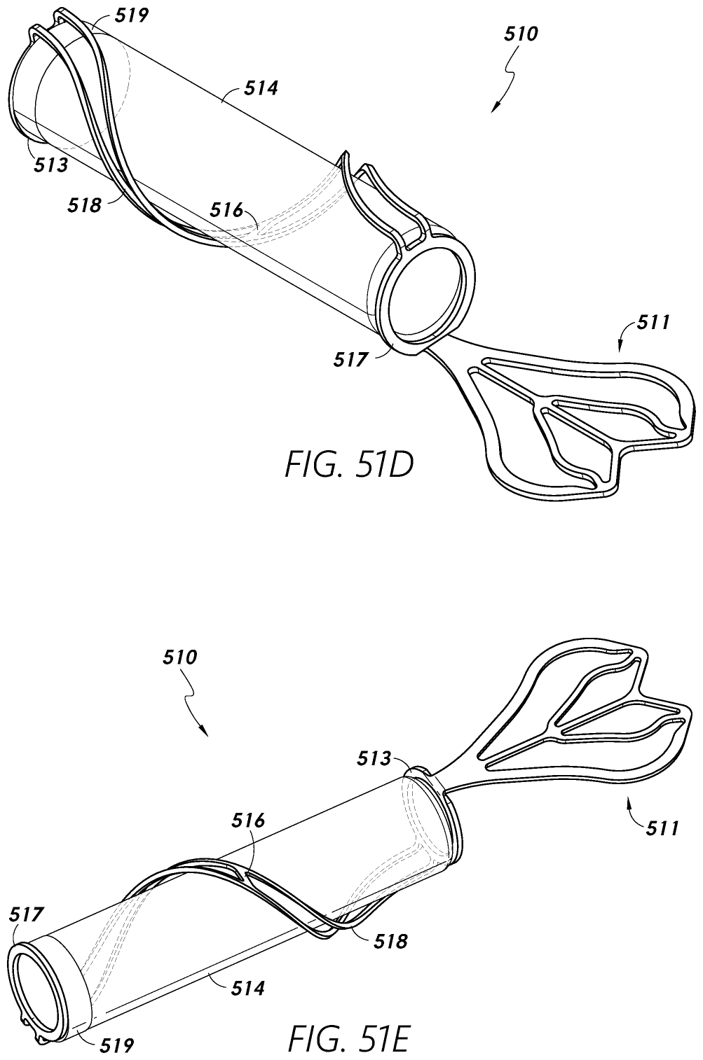

FIGS. 51A through 51E illustrate at least a portion of a medical implant device including a sensor-retention structure 510 including one or more runner-type sensor-support struts 518. FIG. 51A shows the sensor-retention structure 510 in a flattened- or straighten-out configuration, whereas FIGS. 51B through 51E show perspective views of the sensor-retention structure 510 in a sensor-wrapping configuration. The sensor-retention structure 510 can include longitudinal support struts 518 connected between distal 517 and proximal 513 stopper rings. In some embodiments, the sensor-retention structure 510 further includes one or more cross or horizontal struts 516 connected between two longitudinal support struts 518 to provide further mechanical stability for the sensor-retention structure 510. The longitudinal support struts 518 can secure a sensor 514 therein in the illustrated wrapped configuration. In the embodiment of FIGS. 51A through 51E, the longitudinal struts 518 can be connected to the respective stopper rings 513, 517 adjacent to one another on one half of the respective rings' circumference, as shown.

The twisted configuration shown in FIGS. 51B-51E of the sensor-retention structure 510 may be configured in the twisted configuration prior to placement of the sensor 514 in some implementations. In some implementations, the sensor-retention structure 510 can be wrapped/twisted around the sensor 514 after placement of the sensor against the longitudinal struts 518. The amount of wrapping of the strut(s) 518 and/or the length h of the strut(s) 518 can be determined to result in an axial length 12 between the proximal 513 and distal 517 rings that corresponds to a length of the sensor 514.

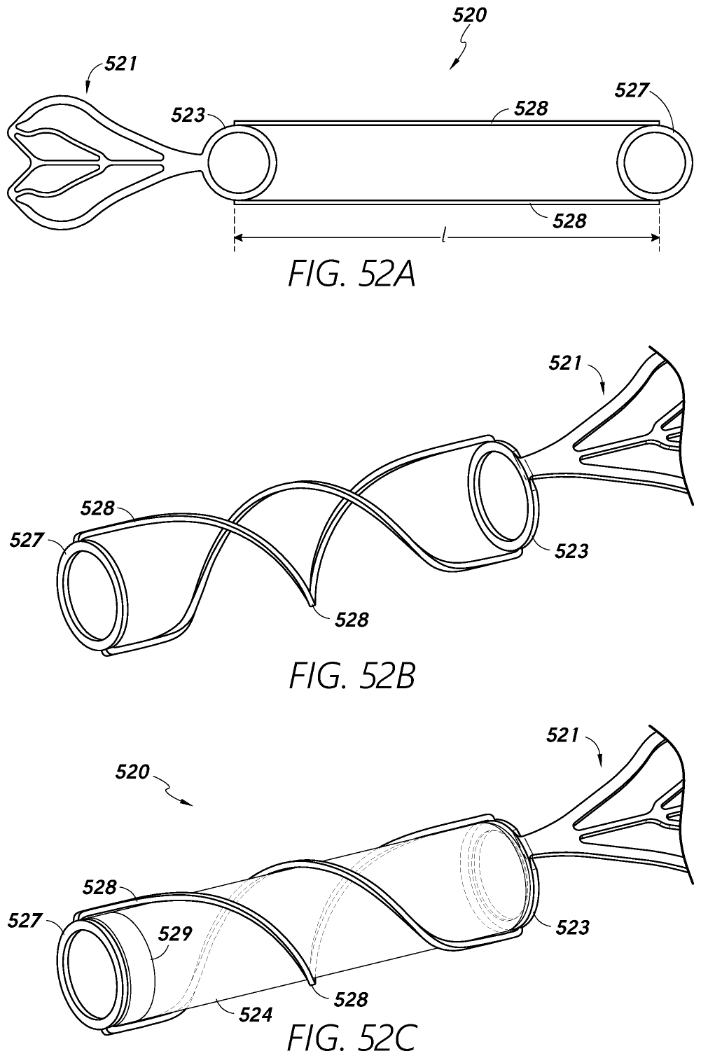

FIGS. 52A through 52C illustrate at least a portion of a medical implant device including a sensor-retention structure 520 including one or more runner-type sensor-support struts 528. FIG. 52A shows the sensor-retention structure 520 in a flattened- or straighten-out configuration, whereas FIGS. 52B and 52C show perspective views of the sensor-retention structure 520 in a sensor-wrapping configuration. The sensor-retention structure 520 can include longitudinal support struts 528 connected between distal 527 and proximal 523 stopper rings. In some embodiments, the sensor-retention structure 520 further includes one or more cross or horizontal struts (not shown) connected between two longitudinal support struts 528 to provide further mechanical stability for the sensor-retention structure 520. The longitudinal support struts 528 can secure a sensor 524 therein in the illustrated wrapped configuration. In the embodiment of FIGS. 52A through 52C, the longitudinal struts 528 can be connected to the respective stopper rings 523, 527 on opposite sides of the rings, as shown.

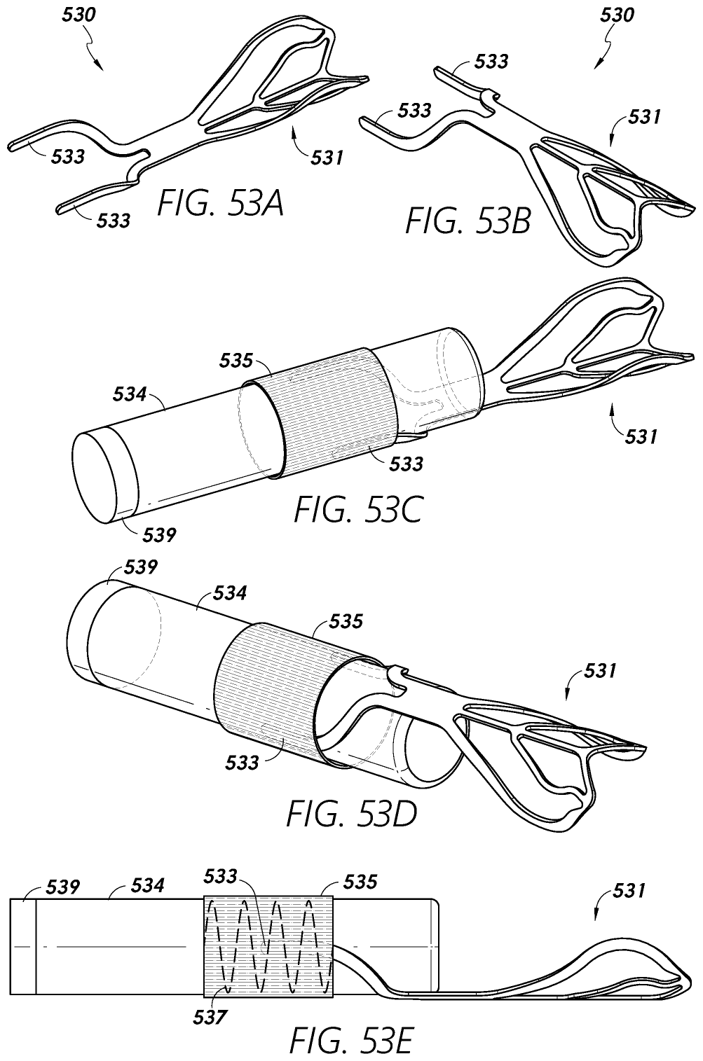

FIGS. 53A through 53E illustrate perspective views of a sensor-retention structure 530 that may be associated with a medical implant device in accordance with one or more embodiments of the present disclosure. The sensor-retention structure 530 includes first and second axially-extending prongs 533. A sensor device 534 may be placed against the prongs 533, wherein a sleeve of any suitable or desirable material 535 may be wrapped at least partially around the sensor 534 and the prongs 533, thereby at least partially securing the sensor 534 to the prongs 533 and therefore to the sensor-retention structure.

With respect to FIG. 53E, in some embodiments, reinforcement wiring 537 may be included inside or outside of the sleeve 535 to further secure the sleeve 535 to the sensor 534 and/or prongs 533. It should be understood that any of the embodiments disclosed herein may have reinforcement wiring wrapped around at least a portion of a sensor and/or sensor-retention structure (e.g., sensor-retention arms or fingers). The reinforcement wiring 537 may comprise a mesh, coil, braid, suture, whether metal or polymer, or any other material. The reinforcement wiring 537 may be a non-metal suture in some embodiments.

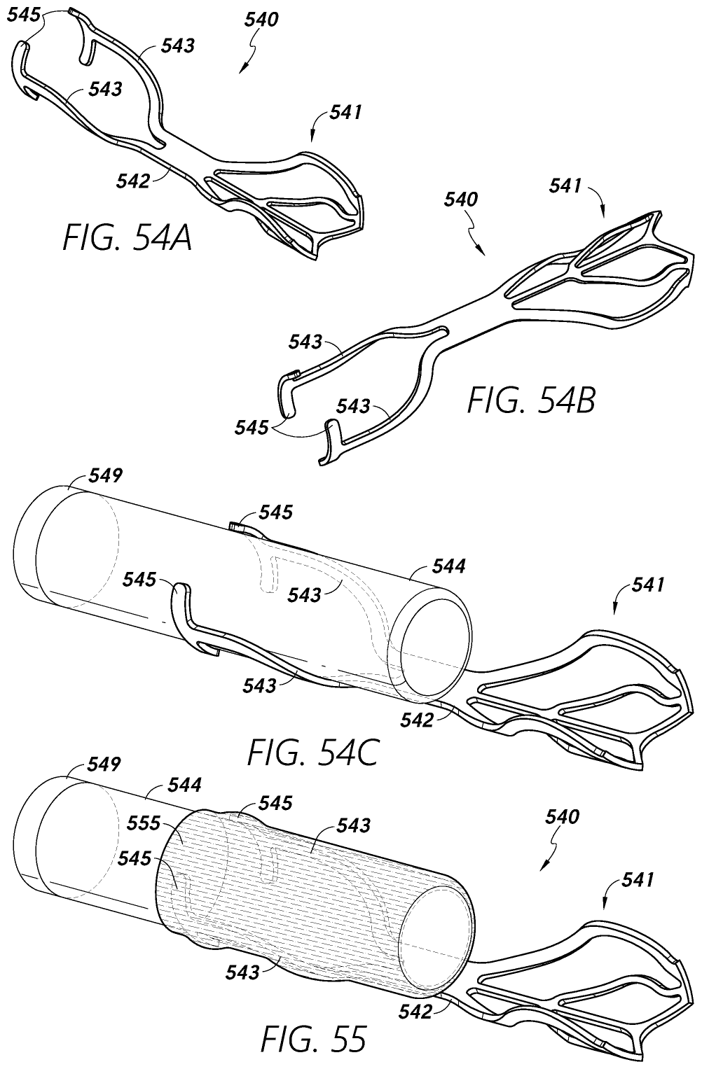

FIGS. 54A through 54C illustrate perspective views of a sensor-retention structure 540 that may be associated with a medical implant device in accordance with one or more embodiments of the present disclosure. The sensor-retention structure 540 includes first and second axially-extending arms 543, each having associated therewith a distal crosspiece 545. A sensor device 544 may be placed against the arms 543 and the crosspieces 545 thereby at least partially securing the sensor 544 to the sensor-retention structure 540.

In FIGS. 54A through 54C, the crosspieces 545 are shown as projecting circumferentially on both sides of the respective arms 543. In some embodiments, the crosspieces project to only one side of the respective arm. That is, while some embodiments may include “T”-shaped sensor-retention arms/forms, some embodiments may include “L”-shaped sensor-retention arms/forms.

FIG. 55 illustrates a perspective view of the sensor-retention structure of FIGS. 54A through 54C, wherein a sleeve of any suitable or desirable material 555 is wrapped at least partially around the sensor 544, and the arms 543, and the crosspieces 545, thereby at least partially securing the sensor 544 to the sensor-retention structure 540. The addition on the crosspieces 545 can enhance the engagement of the sensor-retention structure 540 with the sensor device 544 and/or the sleeve 555. For example, the crosspieces 545 can reduce the risk of the arms 543 sliding out from under the sleeve 555 by increasing the surfaces and/or surface areas against which the sensor-retention structure 540 engages with the sleeve (e.g., polymer, carbothane) 555.

In some implementations, the sleeve 555 is applied around the sensor 544 prior to placement of the sensor 544 against the arms 543. After application/placement of the sleeve material about the sensor 544, the material of the sleeve 555 may be re-flowed to enhance the coupling of the arms 543 and crosspieces 545 with the sleeve 555. For example, re-flowing the sleeve material 555 may involve heating the sleeve material 555 to cause the material to conform to the surfaces of the sensor 544 and sensor-retention structure 540. It should be understood that any of the embodiments disclosed herein may incorporate polymer sleeve/material application to one or more portions thereof, and that such polymer sleeve material may be re-flowed during one or more steps of a manufacturing and/or assembly process to facilitate binding/engagement of the sleeve material with component(s) of the relevant sensor-retention structure.

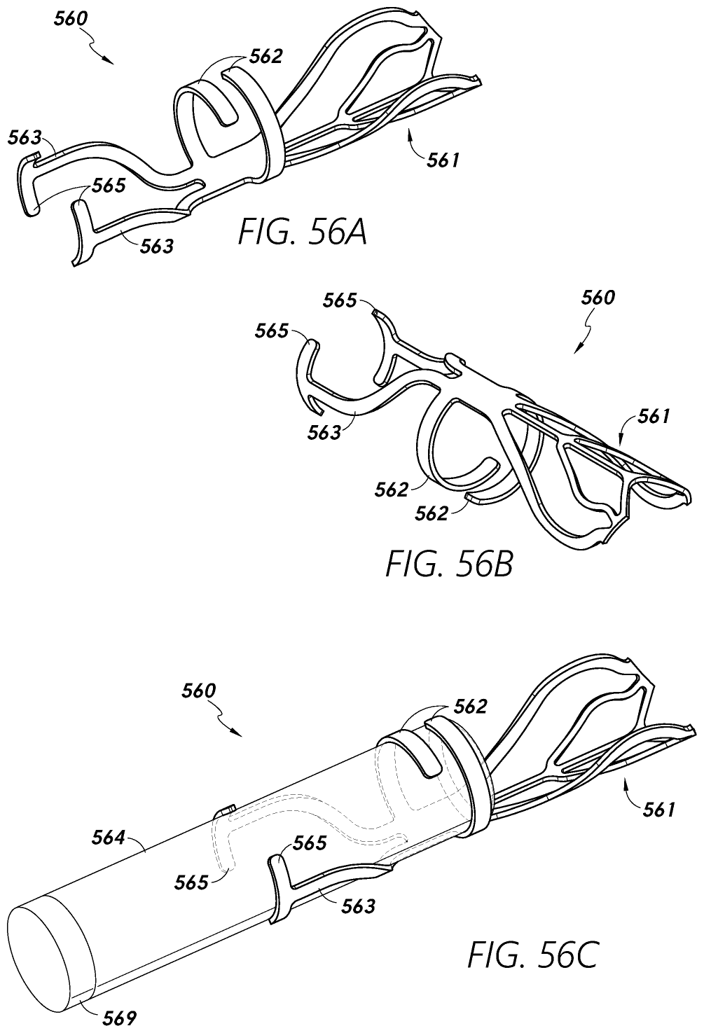

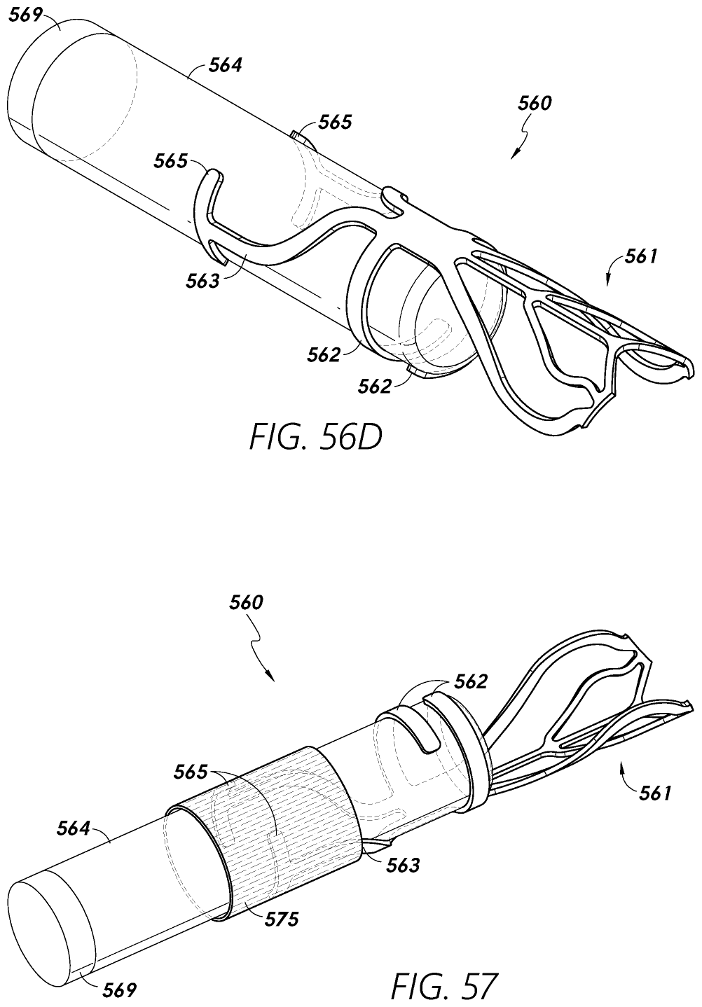

FIGS. 56A through 56D illustrate perspective views of a sensor-retention structure 560 that may be associated with a medical implant device in accordance with one or more embodiments of the present disclosure. The sensor-retention structure 560 includes first and second axially-extending arms 563, each having associated therewith a distal crosspiece 565. The crosspieces 565 can provide additional circumferential surface contact between the sensor-retention structure 560 and the sensor 564.

The sensor-retention structure 560 further includes a plurality of opposing fingers 562 configured to be wrapped or otherwise disposed at least partially around a cylindrical sensor device 564. The sensor device 564 may be placed against the arms 563 and the crosspieces 565 and secured by the arms 563 and the fingers 562 to the sensor-retention structure 560. The fingers 562 may wrap any arc distance around the exterior/circumference of the sensor cylinder 564. Although two opposing, axially-offset fingers 562 are shown, it should be understood that any number and/or orientation of fingers may be included.

FIG. 57 illustrates a perspective view of the sensor-retention structure of FIGS. 56A through 56D, wherein a sleeve of any suitable or desirable material 575 is wrapped at least partially around the sensor 564, and the arms 563, and the crosspieces 565, thereby at least partially securing the sensor 564 to the sensor-retention structure 560. The sensor-retention structure 560 may have any of the features and/or characteristics of the sensor-retention structure 540, including with respect to the sleeve 555 and sleeve 575.

### Sensor-Retention Cage Structures

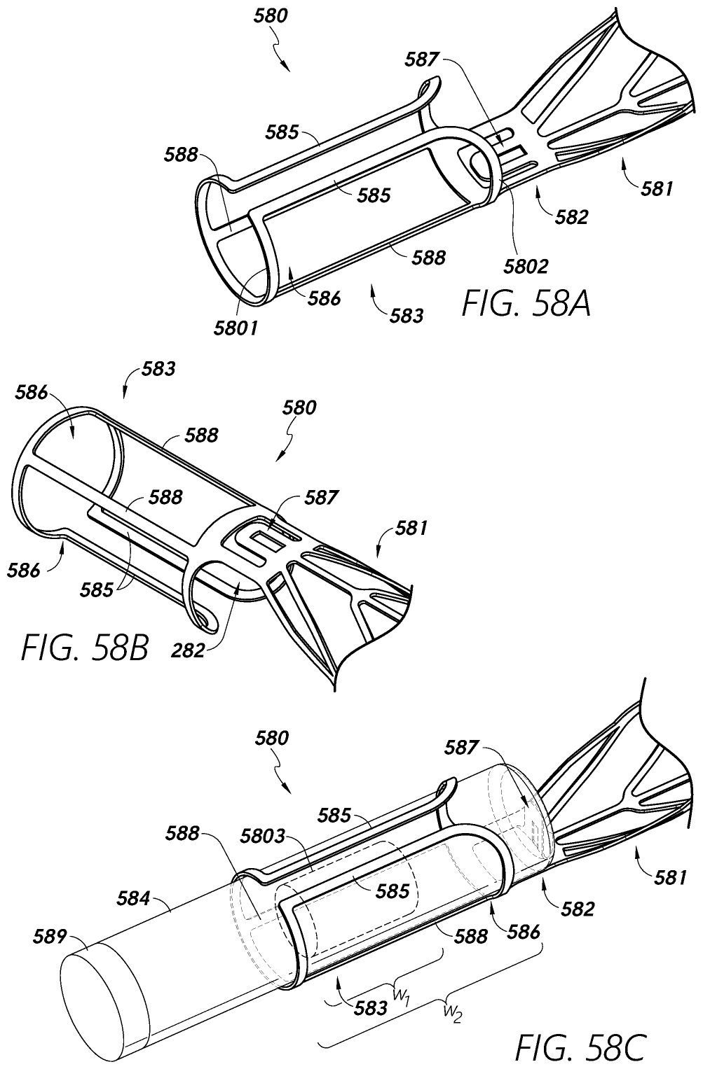

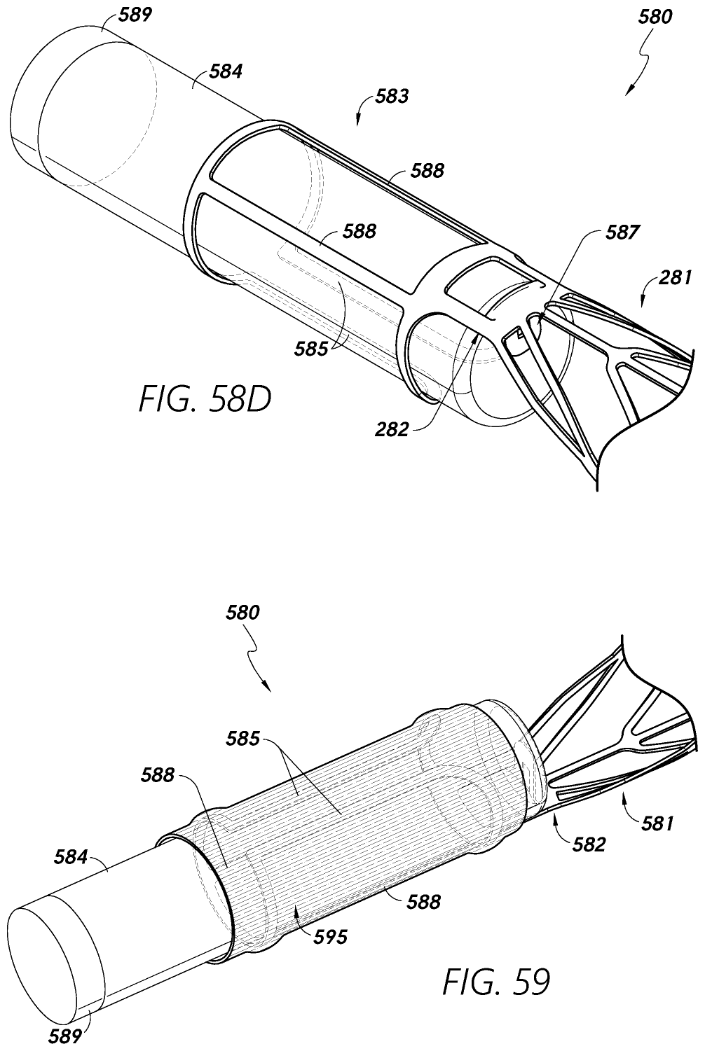

FIGS. 58A through 58D illustrate perspective views of a sensor-retention structure 580 associated with a medical implant device in accordance with one or more embodiments of the present disclosure. The sensor-retention structure 580 includes a cage 583 configured to hold a sensor device 584, such as a cylindrical sensor device in accordance with aspects of the present disclosure. The cage 583 can include a plurality of longitudinal sensor-support struts 588, 585 and a proximal stopper tab 587, which may be configured to be folded or bent inward, as shown, to prevent proximal movement of a sensor device 584 contained within the cage structure 583 proximally beyond an axial position of the tab 587.

The circumferential and axial areas between the longitudinal sensor-support struts 588, 585 and the distal 5801 and proximal 5802 circumferential support struts form windows 586. Such windows 586 can provide openings through which electromagnetic signals may propagate to and/or from a wireless transmission element 5803 of the sensor device 584. For example, in embodiments including sensor-retention structures/cages comprising electrically conductive material, such material can interfere with the transmission of electromagnetic signals. With respect to transmission elements that comprise conductive coil features, as described in detail above, conductive material overlapping axially and/or circumferentially with such coils can result in signal noise caused at least in part by the induction of electrical current therein in the presence of electric and/or magnetic fields associated with wireless data or power transmission between the transmission element 5803 and an external source or receiver. Therefore, the struts 585, 588, 5801, and 5802 may be configured/designed according to dimensions that provide window feature(s) that are wide and/or long enough to not substantially interfere with signal transmissions to/from the transmission element 5803. For example, where the transmission element (e.g., coil antenna) has a length w₁, as shown in FIG. 58C, in some embodiments, the window feature(s) 586 may advantageously have a length dimension w₂ that is greater than the dimension w₁ of the transmission element. Furthermore, the window feature(s) 586 may be configured to axially overlap the transmission element 5803 such that the transmission element fits axially within the window feature(s) 586 when the sensor 584 is retained by the sensor-retention structure, as in FIG. 58C. In some embodiments, the window features 586 axially overlap at least 50% of the axial dimension w₁ of the transmission element 5803. In some embodiments, the length dimension w₂ is at least 50% of the dimension w₁ of the transmission element 5803.

FIG. 59 illustrates a perspective view of the sensor-retention structure 580 of FIGS. 58A through 58D, wherein a sleeve of any suitable or desirable material 595, such as thermoplastic polyurethane (TPU), is wrapped at least partially around the sensor 584, and the struts 585, 585, thereby at least partially securing the sensor 584 to the sensor-retention structure 580.

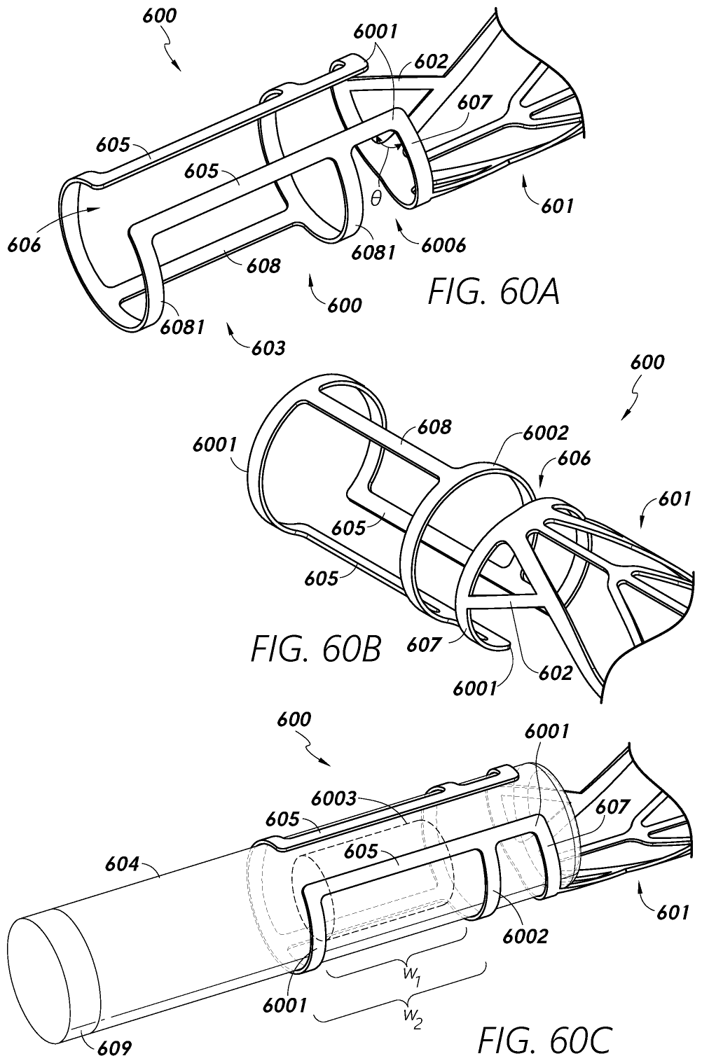

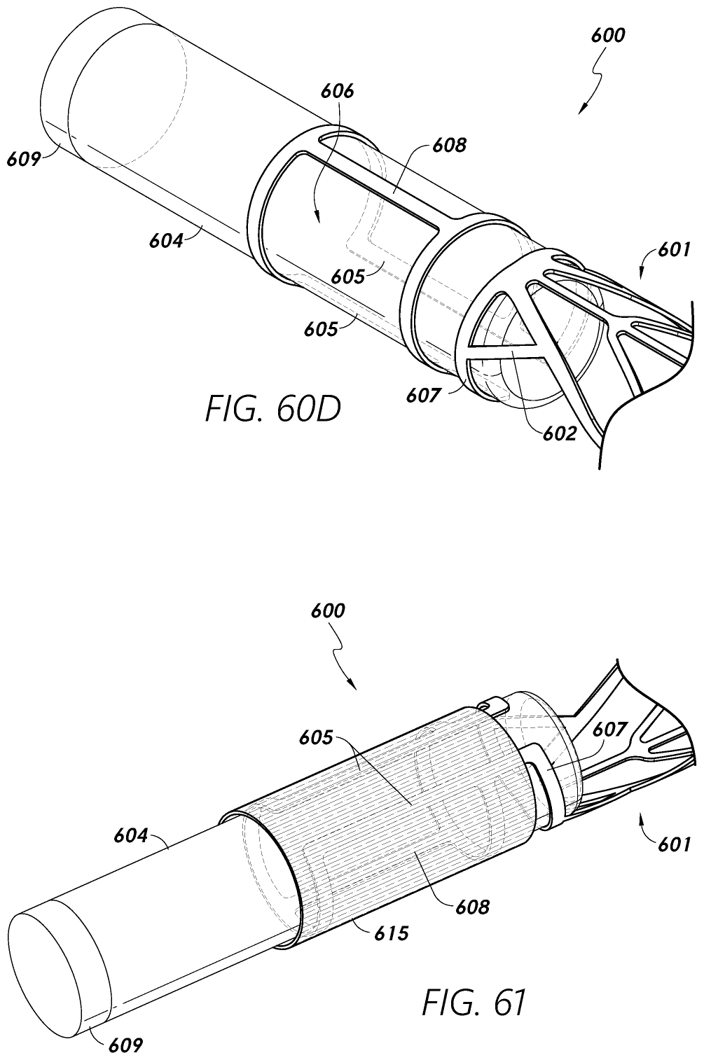

FIGS. 60A through 60D illustrate perspective views of a sensor-retention structure 600 associated with a medical implant device in accordance with one or more embodiments of the present disclosure. The sensor-retention structure 600 includes a cage structure 603 configured to hold a sensor device 604, such as a cylindrical sensor device in accordance with aspects of the present disclosure. The cage 603 can include a plurality of longitudinal sensor-support struts 608, 605. The cage 603 may be coupled to an arm 601 of the medical implant device (e.g., a shunt implant device as described herein), via one or more of the longitudinal struts 605, wherein a gap 6006 is present between the medical implant device arm 601 and the cage structure 603, as shown. In some embodiments, one or more proximally-disposed, distally-angled (with respect to the interface of the struts 602, 607 with the arm 601) support struts 602, 607 may provide mechanical stability for the sensor-retention structure 600, as shown.

The circumferential and axial areas between the longitudinal sensor-support struts 608, 605 and the distal 6001 and proximal 6002 circumferential support struts form windows 606. Such windows 606 can provide openings through which electromagnetic signals may propagate to and/or from a wireless transmission element 6003 of the sensor device 604. For example, in embodiments including sensor-retention structures/cages comprising electrically conductive material, such material can interfere with the transmission of electromagnetic signals. With respect to transmission elements that comprise conductive coil features, as described in detail above, conductive material overlapping axially and/or circumferentially with such coils can result in signal noise caused at least in part by the induction of electrical current therein in the presence of electric and/or magnetic fields associated with wireless data or power transmission between the transmission element 6003 and an external source or receiver. Therefore, the struts 605, 608, 6001, and 6002 may be configured/designed according to dimensions that provide window feature(s) that are wide and/or long enough to not substantially interfere with signal transmissions to/from the transmission element 6003. For example, where the transmission element (e.g., coil antenna) has a length w₁, as shown in FIG. 60C, in some embodiments, the window feature(s) 606 may advantageously have a length dimension w₂ that is greater than the dimension w₁ of the transmission element. Furthermore, the window feature(s) 606 may be configured to axially overlap the transmission element 6003 such that the transmission element fits axially within the window feature(s) 606 when the sensor 604 is retained by the sensor-retention structure, as in FIG. 60C. In some embodiments, the window feature(s) 606 axially overlap at least 50% of the axial dimension w₁ of the transmission element 6003. In some embodiments, the length dimension w₂ is at least 50% of the dimension w₁ of the transmission element 6003.

FIG. 61 illustrates a perspective view of the sensor-retention structure 600 of FIGS. 60A through 60D, wherein a sleeve 615 of any suitable or desirable material, such as thermoplastic polyurethane (TPU), is wrapped at least partially around the sensor 604, and the arms 608, 605, thereby at least partially securing the sensor 604 to the sensor-retention structure 600.

The sensor-retention structure 600 includes a hinge point 6001 where the distally-angled support strut 607 bends at the intersection of the distally-angled support strut 607 and the longitudinal strut 605. The hinge point 6001, as configured in FIGS. 60A through 60D, can serve to guide an outer sheath of a delivery system in which the implant device associated with the sensor-retention structure 600 is delivered to the target location over other structure of the sensor-retention structure 600, such as structure of the cage 603 that is distal to the hinge point 6001. Therefore, the hinge point 6001 can provide a hinge to prevent overloading of the interface between the sleeve 615 and the sensor-retention structure 600, which might otherwise be inclined to cause the outer sheath of the delivery system in which the implant device is transported to become caught on the sleeve material 615 when placing the sensor-retention structure 600 into the delivery system. The angle θ of the support strut 607 at the joining of the strut 607 with the longitudinal strut 605 can advantageously be greater than 90°, as shown, in order to reduce the risk of the hinge point 6001 becoming caught on certain types of delivery system sheaths.

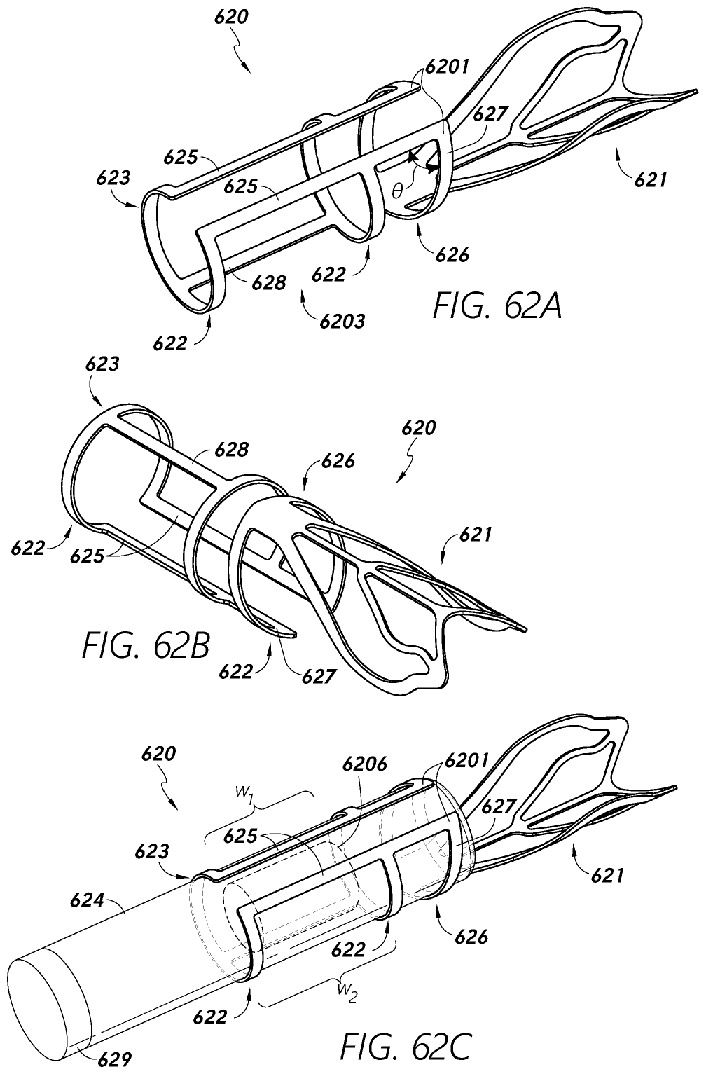

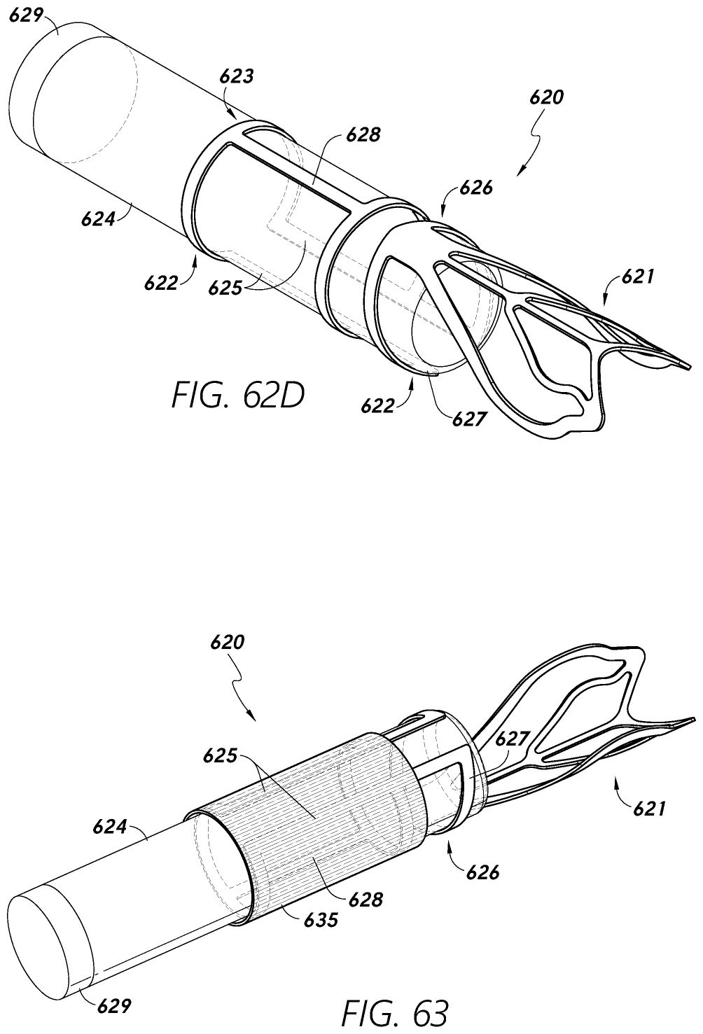

FIGS. 62A through 62D illustrate perspective views of a sensor-retention structure 620 associated with a medical implant device in accordance with one or more embodiments of the present disclosure. The sensor-retention structure 620 includes a cage structure 623 configured to hold a sensor device 624, such as a cylindrical sensor device in accordance with aspects of the present disclosure. The cage 623 can include a plurality of longitudinal sensor-support struts 628, 625. The cage 623 may be coupled to an arm 621 of the medical implant device (e.g., a shunt implant device as described herein), via one or more of the longitudinal struts 625. In some embodiments, the cage includes one or more proximally-disposed, proximally-angled (with respect to the interface of the struts 627 with the arm 621) support struts 627 forming a generally distally-pointing point/arrow, as shown.

The circumferential and axial areas between the longitudinal sensor-support struts 628, 625 and the distal and proximal circumferential support struts 622 form windows 6203. Such windows 6203 can provide openings through which electromagnetic signals may propagate to and/or from a wireless transmission element 6206 of the sensor device 624. The struts 625, 628, and 622 may be configured/designed according to dimensions that provide window feature(s) that are wide and/or long enough to not substantially interfere with signal transmissions to/from the transmission element 6206. For example, where the transmission element (e.g., coil antenna) has a length w₁, as shown in FIG. 62C, in some embodiments, the window feature(s) 6203 may advantageously have a length dimension w₂ that is greater than the dimension w₁ of the transmission element. Furthermore, the window feature(s) 6203 may be configured to axially overlap the transmission element 6206 such that the transmission element fits axially within the window feature(s) 6203 when the sensor 624 is retained by the sensor-retention structure, as in FIG. 62C. In some embodiments, the window feature(s) 6203 axially overlap at least 50% of the axial dimension w₁ of the transmission element 6206. In some embodiments, the length dimension w₂ is at least 50% of the dimension w₁ of the transmission element 6206.

FIG. 63 illustrates a perspective view of the sensor-retention structure 620 of FIGS. 62A through 62D, wherein a sleeve 635 of any suitable or desirable material, such as thermoplastic polyurethane (TPU), is wrapped at least partially around a sensor 624, and the struts 628, 625, thereby at least partially securing the sensor 624 to the sensor-retention structure 620.

The sensor-retention structure 620 includes a hinge point 6201 where the proximally-angled support strut 627 bends at the intersection of the proximally-angled support strut 627 and the longitudinal strut 625. The hinge point 6201, as configured in FIGS. 62A through 62D, can serve to guide an outer sheath of a delivery system in which the implant device associated with the sensor-retention structure 620 is delivered to the target location over other structure of the sensor-retention structure 620, such as structure of the cage 6203 that is distal to the hinge point 6201. Therefore, the hinge point 6201 can provide a hinge to prevent overloading of the interface between the sleeve 635 and the sensor-retention structure 620, which might otherwise be inclined to cause the outer sheath of the delivery system in which the implant device is transported to become caught on the sleeve material 635 when placing the sensor-retention structure 620 into the delivery system. The angle θ of the support strut 627 at the joining of the strut 627 with the longitudinal strut 625 can advantageously be less than 90°, as shown, in order to reduce the risk of the hinge point 6201 becoming caught on certain types of delivery system sheaths.

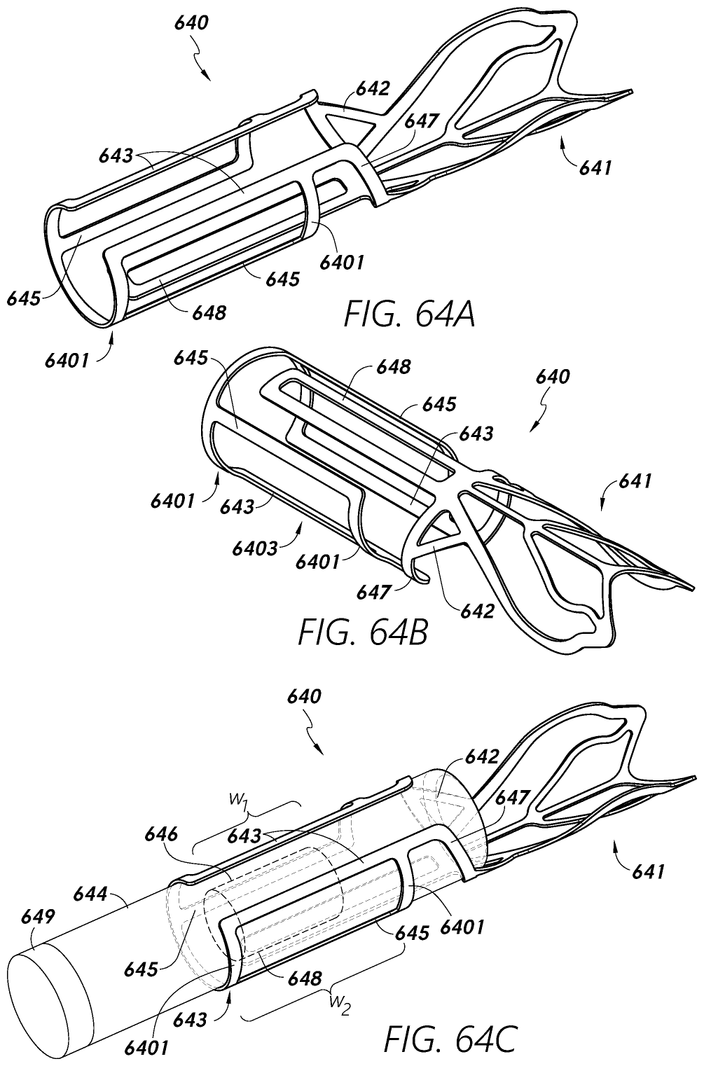

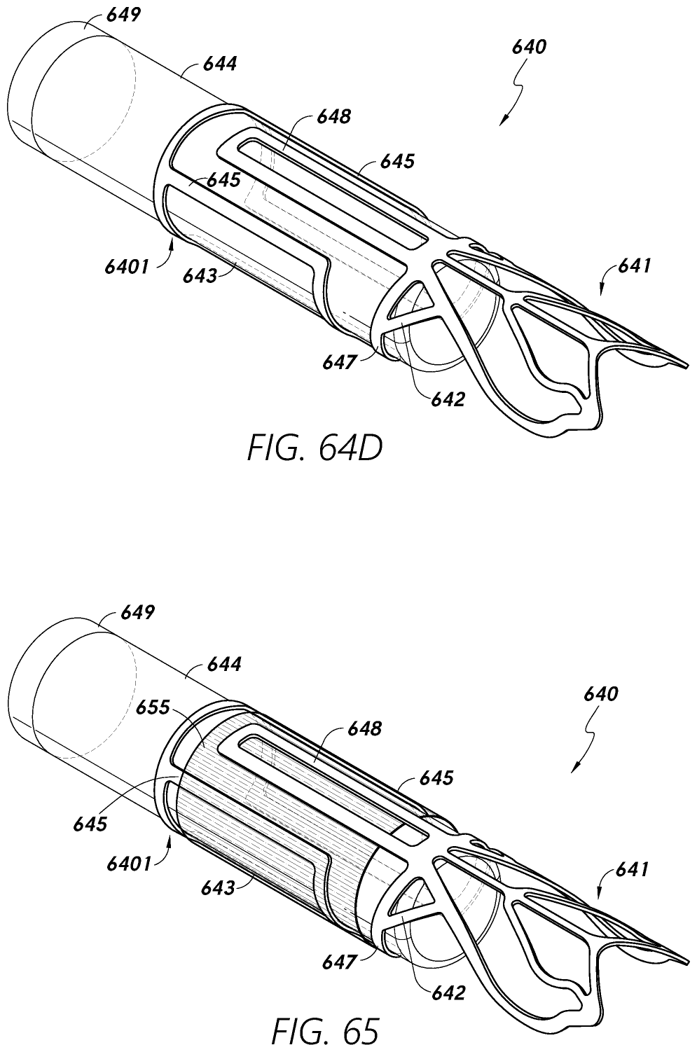

FIGS. 64A through 64D illustrate perspective views of a sensor-retention structure 640 associated with a medical implant device in accordance with one or more embodiments of the present disclosure. The sensor-retention structure 640 includes a cage structure 643 configured to hold a sensor device 644 (see FIGS. 64C and 64D), such as a cylindrical sensor device in accordance with aspects of the present disclosure. The cage 643 can include a plurality of longitudinal sensor-support struts 643, 645. The cage 643 can advantageously also include a longitudinal support structure 648, which may have a multi-strut and/or loop form/configuration. The cage 643 may be coupled to an arm 641 of the medical implant device (e.g., a shunt implant device as described herein), via one or more of the longitudinal struts 643. In some embodiments, one or more proximally-disposed, distally-angled support struts 642, 647 may provide mechanical stability for the sensor-retention structure 640, as shown.

The circumferential and axial areas between the longitudinal sensor-support struts 643, 645 and the distal and proximal circumferential support struts 6401 form windows 6403. Such windows 6403 can provide openings through which electromagnetic signals may propagate to and/or from a wireless transmission element 646 of the sensor device 644 and reduce interference/noise associated therewith. The struts 645, 643, and 6401 may be configured/designed according to dimensions that provide window feature(s) that are wide and/or long enough to not substantially interfere with signal transmissions to/from the transmission element 646. For example, where the transmission element (e.g., coil antenna) has a length w₁, as shown in FIG. 64C, in some embodiments, the window feature(s) 6403 may advantageously have a length dimension w₂ that is greater than the dimension w₁ of the transmission element 646. Furthermore, the window feature(s) 6403 may be configured to axially overlap the transmission element 646 such that the transmission element fits axially within the window feature(s) 6403 when the sensor 644 is retained by the sensor-retention structure, as in FIG. 64C. In some embodiments, the window feature(s) 6403 axially overlap at least 50% of the axial dimension w₁ of the transmission element 646. In some embodiments, the length dimension w₂ is at least 50% of the dimension w₁ of the transmission element 646.

FIG. 65 illustrates a perspective view of the sensor-retention structure 640 of FIGS. 64A through 64D, wherein a sleeve 655 of any suitable or desirable material, such as thermoplastic polyurethane (TPU), is wrapped at least partially around the sensor 644, the struts 643, 645, and the longitudinal support structure, thereby at least partially securing the sensor 644 to the sensor-retention structure 640. In some embodiments, the longitudinal support structure 648 may be disposed over/outside the sleeve 655 to provide sheathability for the sensor-retention structure 640.

In some embodiments, the longitudinal support structure 648 is disposed substantially on the outside of the sleeve/material 655, while other struts and/or portions of the sensor-retention structure 640 are disposed at least partially between the sleeve/material 655 and the sensor cylinder 644. Alternatively, the longitudinal support structure 648 may be tucked under the sleeve/material 655, whereas other struts and/or portions of the sensor-retention structure 640 are disposed on the outside of the sleeve/material 655.

In some implementations, the longitudinal support structure 648 may be bent away from the axis of the structure 640 (e.g., folded/pulled up) while the sleeve/material 655 is applied to the sensor 644 and to the sensor-retention structure 640, such that the longitudinal support structure 648 is not covered by the sleeve/material 655 (e.g., polymer; carbothane). After the sleeve/material 655 is applied to the sensor 644 and portions of the sensor-retention structure (e.g., portions of struts 643), the longitudinal support structure 648 may be released or otherwise placed onto the sleeve/material 655, such that the longitudinal support structure 648 is disposed generally on or near an outer surface thereof. After the longitudinal support structure 648 has been placed in contact with the outside of the sleeve/material 655, the material 655 may be re-flowed to improve the connection between the sleeve/material 655 and the sensor-retention structure 640.

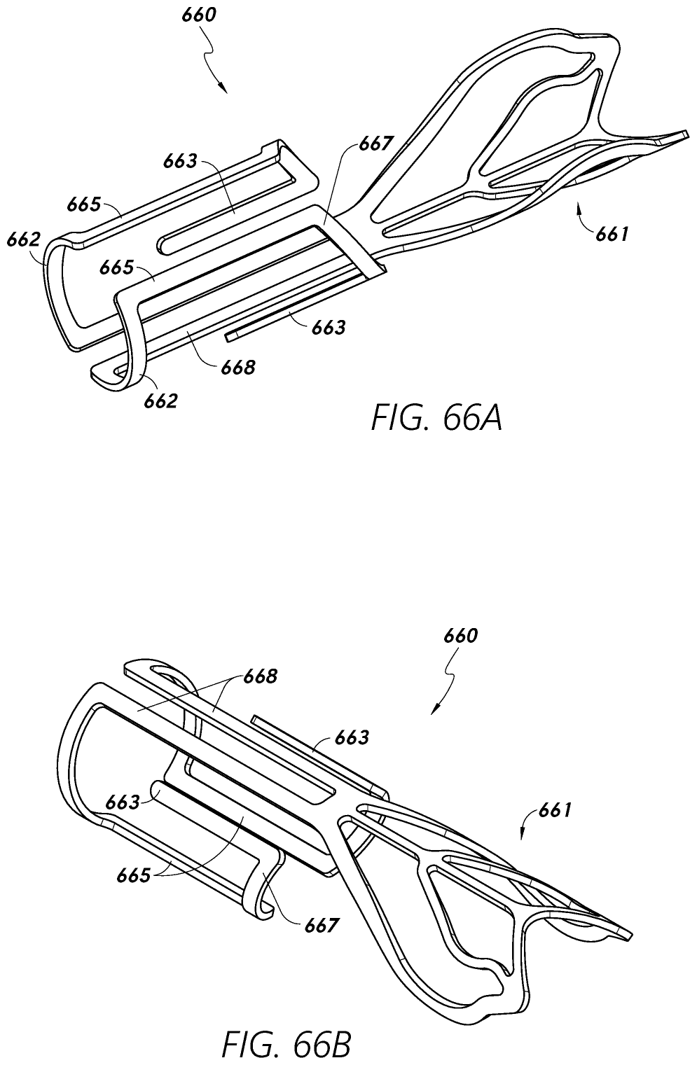

FIGS. 66A and 66B illustrate perspective views of a sensor-retention structure 660 associated with a medical implant device in accordance with one or more embodiments of the present disclosure. The sensor-retention structure 660 includes two support struts 668 extending from an arm 661 of a medical implant device and providing a backside sensor support in accordance with embodiments of the present disclosure. The support struts 668 extend from the arm 661 distally to a distal end of the structure 660, at which point the struts form a bend and continue circumferentially around circumferential support paths 662 generally to a frontside of the sensor-retention structure 660. The struts 665 can further extend proximally generally on the frontside of the structure 660 and again bends to form proximally- and/or circumferentially-angled/projecting strut portions 667. The strut portions 667 can form another bend on respective side portions of the structure 660 and terminate in distally-projecting strut portions 663, as shown.

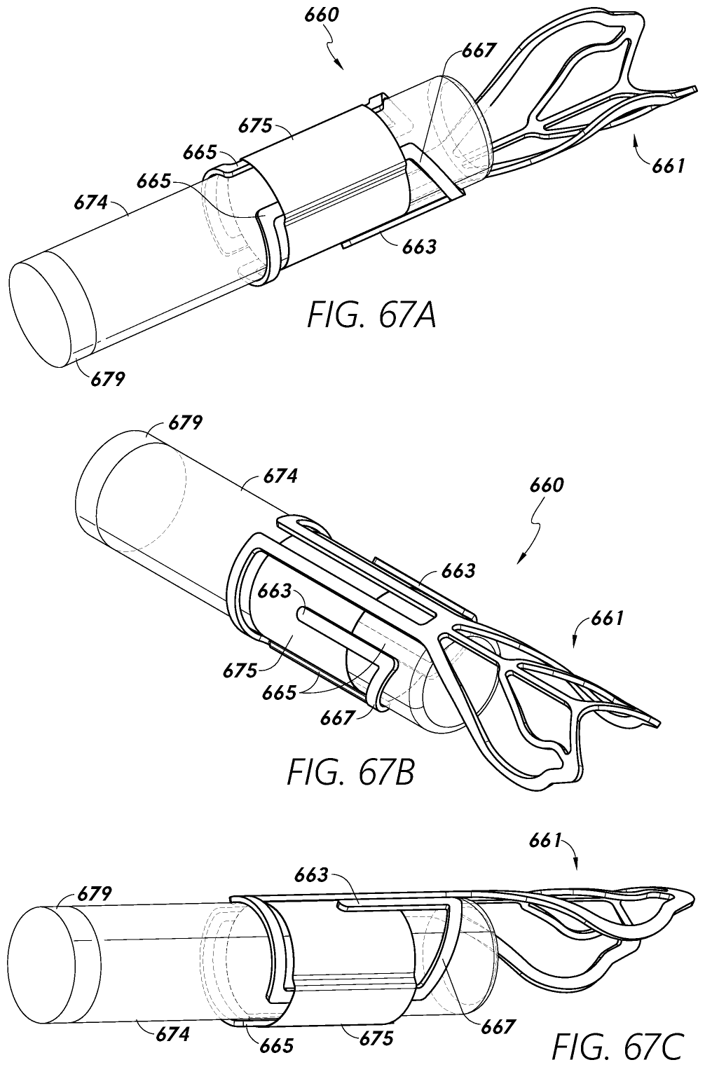

FIG. 67A through 67C illustrates perspective views of the sensor-retention structure 660 of FIGS. 66A and 66B with a sensor device 674 retained therein, wherein a sleeve 675 of any suitable or desirable material, such as thermoplastic polyurethane (TPU), is disposed at least partially around the sensor device 674, the struts 665, and/or the struts 667, and/or the struts 663. In some embodiments, the sleeve 675 is disposed around the sensor device 674 but within one or more of the struts 667 and/or the struts 663.

The sleeve 675 may comprise carbothane or another polymer material. The sleeve 675 can be applied to the sensor 674 and/or strut(s) 665 and/or the strut(s) 665 can be inserted between portions of the sleeve 675 and the sensor 674 in such a manner as to result in the sleeve 675 being deformed and/or otherwise configured to conform around one or more portions of the strut(s) 665, as shown. As with any of the embodiments of the present disclosure, in some implementations, the sensor-retention structure 660 may be loaded into a delivery system sheath prior to delivery to the target implantation site. When deployed, the sensor-retention structure 660 may be unsheathed to allow for deployment. The end strut portion 663 may be placed/disposed outside of the sleeve 675, as shown, and may facilitate sliding of the sensor-retention structure 660 into a delivery sheath or other delivery device. When wrapping the strut portion 663 outside of the sleeve 675, the strut portion 663 may be withdrawn from behind/underneath the sleeve 675, wherein the shape memory characteristics of the strut portions 667, 663 may be such as to cause the strut portion 663 to assume a position axially overlapping the sleeve 675 with respect to the axis of the sensor 674. For example, the struts may comprise Nitinol or other material having super-elasticity allowing for such deformation and positioning of the various strut portions.

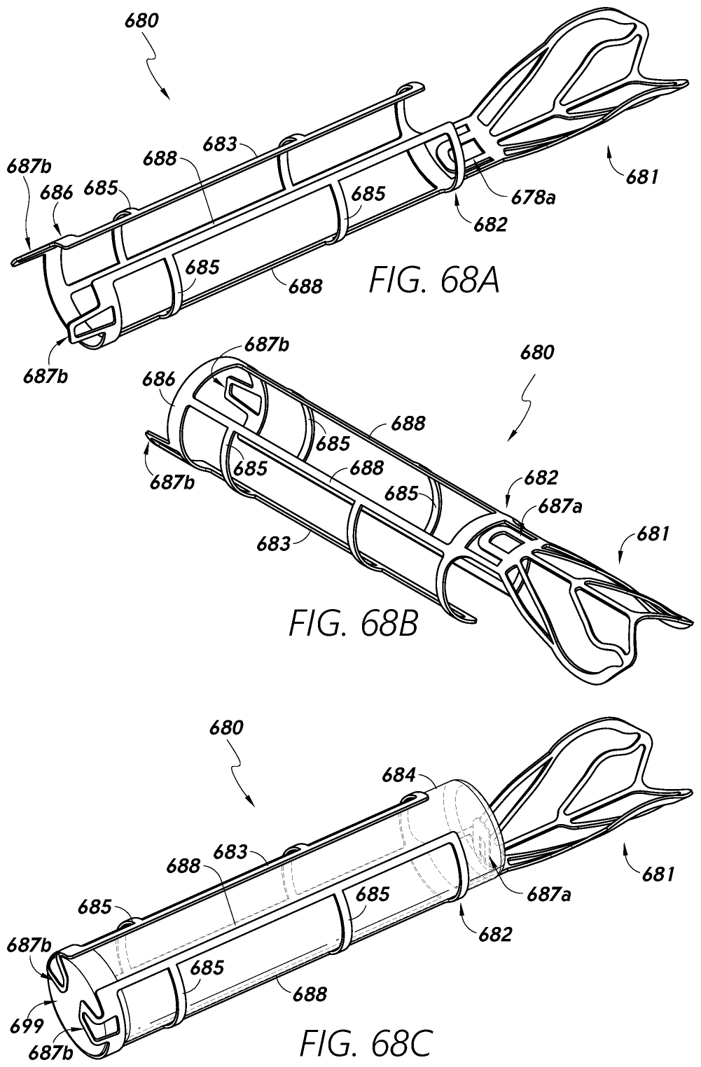

FIGS. 68A through 68C illustrate perspective views of a sensor-retention structure 680 associated with a medical implant device in accordance with one or more embodiments of the present disclosure. The sensor-retention structure 680 includes a cage structure configured to hold a sensor device 684 (see FIG. 68C), such as a cylindrical sensor device in accordance with aspects of the present disclosure. The structure 680 can include a plurality of longitudinal sensor-support struts 688, 683 and one or more distal stopper tabs 687b and/or one or more proximal stopper tabs 687a, which may be configured to be folded or bent inward, as shown, to prevent distal and proximal movement, respectively, of a sensor device 694 contained within the cage structure 680 beyond respective axial positions of the tab(s) 687. The illustrated configurations of the tabs 687 may be achieved through shape memory characteristics of such features, which may be shape-set to bend/project towards the axial center of the sensor device 684 and/or cage structure of the sensor-retention structure 680. The structure 680 can further include a plurality of circumferential struts 685 that provide mechanical stability between the backside longitudinal struts 688 and the frontside longitudinal struts 683. The cage structure 680 may or may not have polymer sleeve material applied thereto to secure the structure 680 to the sensor 684.

The strut 688 that connects the proximal portion/ring 682 to the distal portion/ring 686 can facilitate sheathing of the sensor-retention structure 680, and may further help with retention by providing lengthwise surface area for contact with the sensor device 684 and/or polymer sleeve material that may be applied to the structure 680 and/or sensor device 684. The cage of the sensor-retention structure 680 may include struts that are configured and/or dimensioned to provide windows, as described in detail herein, that align with at least a portion of a transmission element of the sensor 684 to reduce interference/noise with respect to wireless data and/or power transmissions between the transmission element and an external receiver/transmitter.

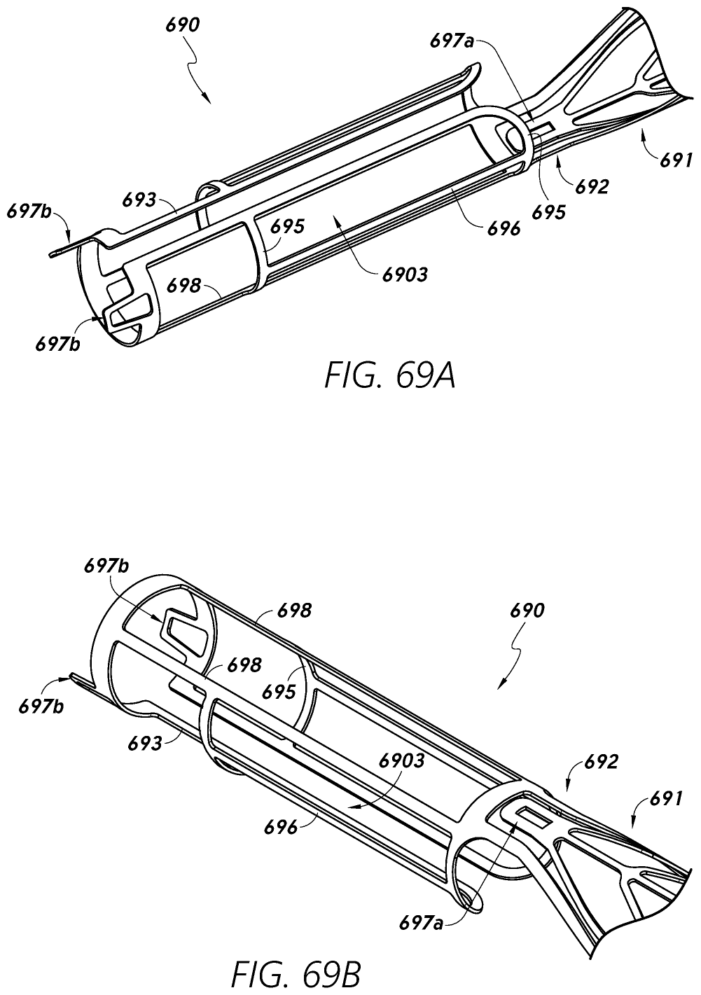

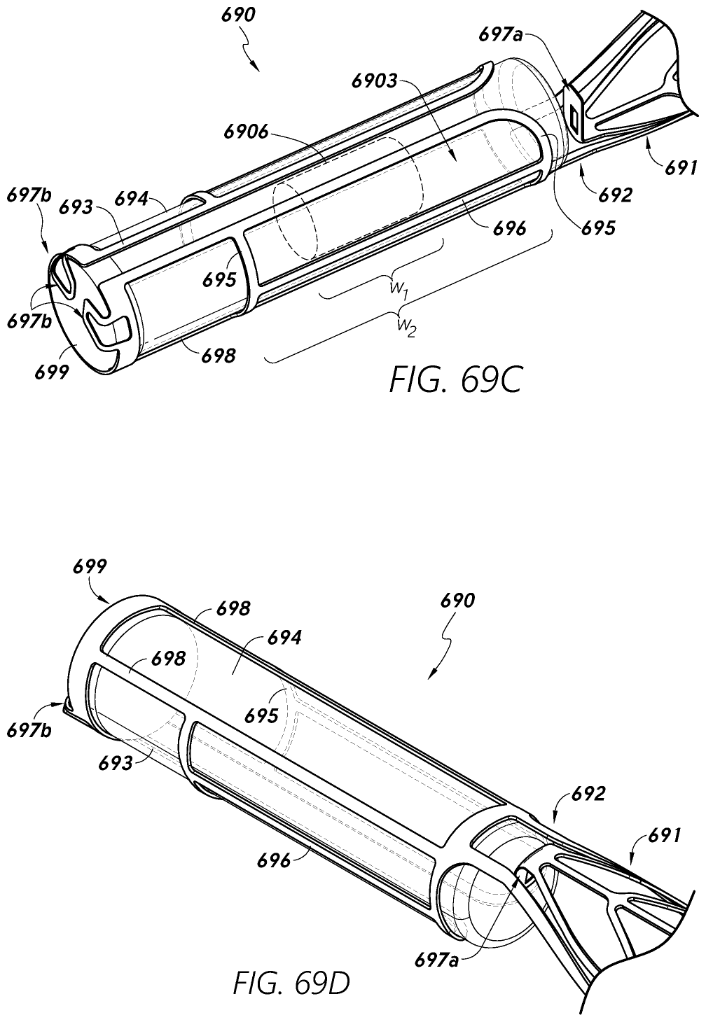

FIGS. 69A through 69D illustrate perspective views of a sensor-retention structure 690 associated with a medical implant device in accordance with one or more embodiments of the present disclosure. The sensor-retention structure 690 includes a cage structure configured to hold a sensor device 694 (see FIGS. 69C and 69D), such as a cylindrical sensor device in accordance with aspects of the present disclosure. The structure 690 can include a plurality of longitudinal sensor-support struts 698, 693 and one or more distal stopper tabs 697b and/or one or more proximal stopper tabs 697a, which may be configured to be folded or bent inward, as shown, to prevent distal and proximal movement, respectively, of a sensor device 694 contained within the cage structure 690 beyond respective axial positions of the tab(s) 697. The structure 690 can further include one or more circumferential struts 695 and/or more intermediate longitudinal struts 696 that provide mechanical stability for the structure 690.

Compared to the embodiment illustrated in FIGS. 68A through 68C, the cage structure 690 can have one less circumferential ring along a length of the structure 690. For example, the removed ring may be generally on a proximal half/side of the cage structure 690, which may correspond to a position of an electrical coupling component of the sensor 694. Generally, circumferential overlap of the electrical coupling component, which may generally run parallel to the coil windings of the electrical coupling, may be more problematic than lengthwise overlapping (e.g., orthogonal to the windings/coil of the electrical coupling component), as with the longitudinal strut 696, which is provided in place of the circumferential strut 685 of the structure 680 in FIGS. 68A through 68C.

The circumferential and axial areas between the longitudinal sensor-support struts 693, 698 and the distal and proximal circumferential support struts 695 form windows 6903. Such windows 6903 can provide openings through which electromagnetic signals may propagate to and/or from a wireless transmission element 6906 of the sensor device 694 and reduce interference/noise associated therewith. The struts 698, 693, and 695 may be configured/designed according to dimensions that provide window feature(s) that are wide and/or long enough to not substantially interfere with signal transmissions to/from the transmission element 6906. For example, where the transmission element (e.g., coil antenna) has a length w₁, as shown in FIG. 69C, in some embodiments, the window feature(s) 6903 may advantageously have a length dimension w₂ that is greater than the dimension w₁ of the transmission element 6906. Furthermore, the window feature(s) 6903 may be configured to axially overlap the transmission element 6906 such that the transmission element fits axially within the window feature(s) 6903 when the sensor 694 is retained by the sensor-retention structure, as in FIG. 69C. In some embodiments, the window feature(s) 6903 axially overlap at least 50% of the axial dimension w₁ of the transmission element 6906. In some embodiments, the length dimension w₂ is at least 50% of the dimension w₁ of the transmission element 6906.

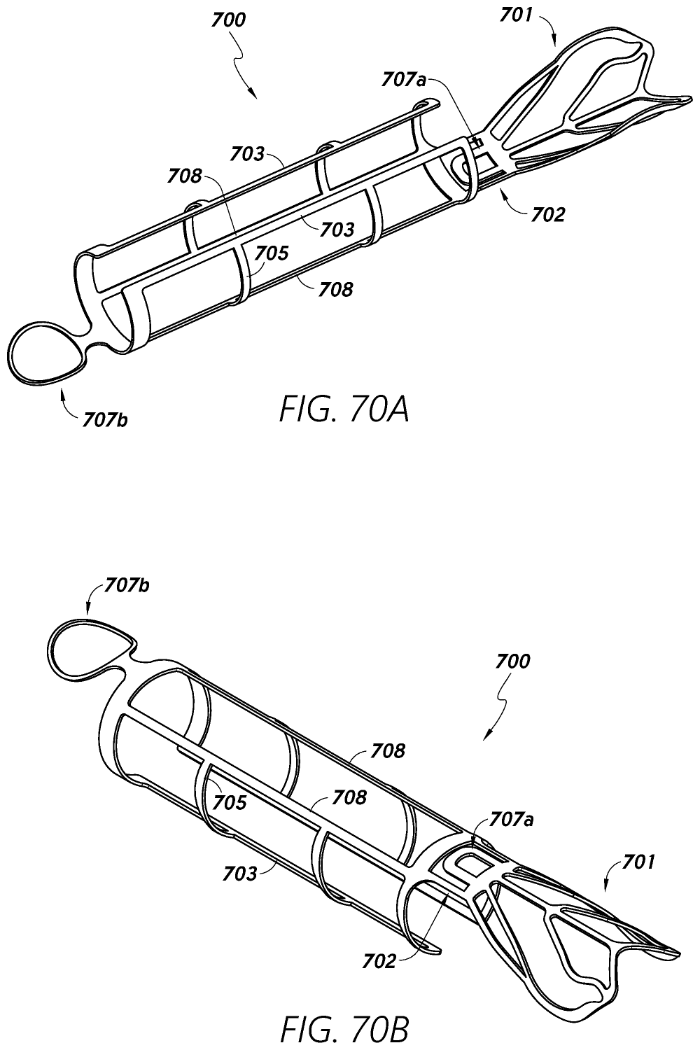

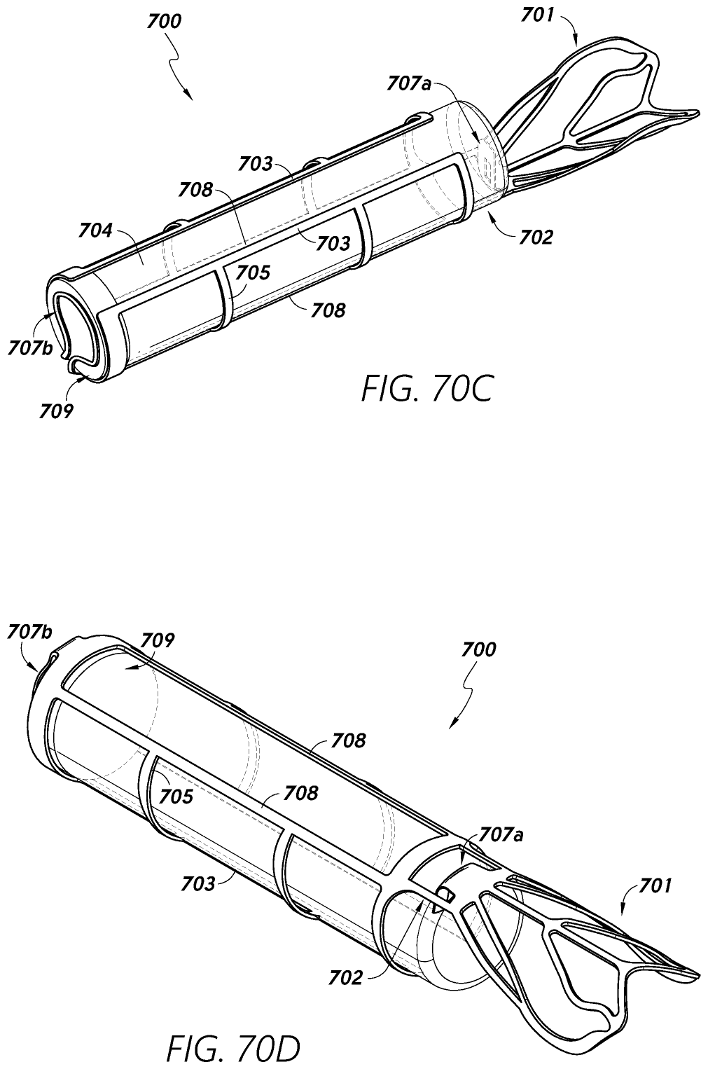

FIGS. 70A through 70D illustrate perspective views of a sensor-retention structure 700 associated with a medical implant device in accordance with one or more embodiments of the present disclosure. The sensor-retention structure 700 includes a cage structure configured to hold a sensor device 704 (see FIGS. 70C and 70D), such as a cylindrical sensor device in accordance with aspects of the present disclosure. The structure 700 can include a plurality of longitudinal sensor-support struts 708, 703 and one or more distal stopper rings 707b and/or one or more proximal stopper tabs 707a, which may be configured to be folded or bent inward, as shown, to prevent distal and proximal movement, respectively, of a sensor device 704 contained within the cage structure 700 beyond respective axial positions of the stopper feature(s) 707. The structure 700 can further include a plurality of circumferential struts 705 that provide mechanical stability between the backside longitudinal struts 708 and the frontside longitudinal struts 703.

The ring stopper 707b may advantageously provide for contact with the sensor element 709 only around a periphery thereof. Such contact may result in a reduced impact on sensor function and/or reduced risk of damage to the sensor element 709 compared to tabs that jut radially inward over the surface (e.g., hermetic seal) of the sensor element.

The ring stopper 707b is shown has having slight curvature corresponding to a curved (e.g., tubular) sheet from which the sensor-retention structure 700 may be cut during manufacturing. In some implementations, the curvature of the ring stopper 707b may be flattened-out in connection with shape setting thereof. Flattening of the ring stopper 707b can serve to increase the surface contact area of the ring stopper 707b on the sensor element 709, thereby potentially spreading-out/distributing the contact load thereon. In some embodiments, the ring stopper 707b is designed to be a similar/same size, or slightly larger, as a hermetic seal of the sensor element 709, thereby reducing impact thereon.

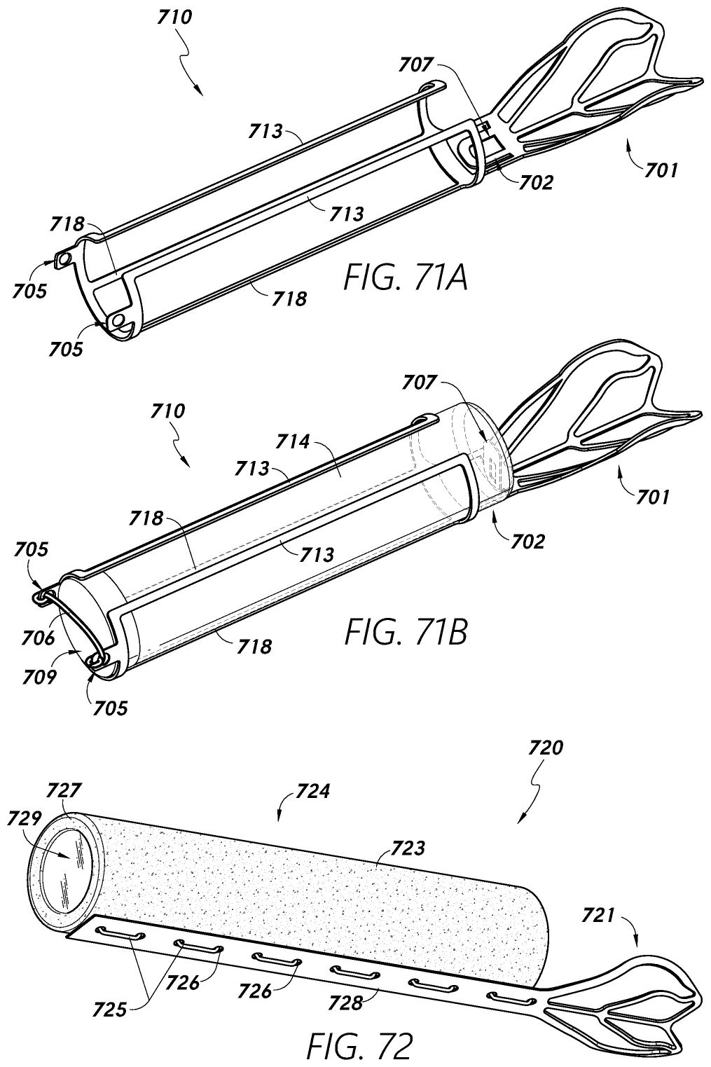

FIGS. 71A and 71B illustrate perspective views of a sensor-retention structure 710 associated with a medical implant device in accordance with one or more embodiments of the present disclosure. The sensor-retention structure 710 includes a cage structure configured to hold a sensor device 704 (see FIG. 71B), such as a cylindrical sensor device in accordance with aspects of the present disclosure. The structure 710 can include a plurality of longitudinal sensor-support struts 718, 713 and one or more distal 705 and/or proximal 702 stopper features. For example, the distal stopper features 705 can include a plurality of projections and/or apertures 705, which may have a suture 706 or other structure/form disposed at least partly therethrough, wherein the suture (or other structure/form) can cross over at least a portion of a diameter of a sensor device 704 disposed at least partially within the cage structure 710 to thereby at least partially interfere with or prevent the sensor device 704 from sliding distally the distal end of the cage structure 710, the stopper features 705, and/or the suture 706.

The structure 710 can further include one or more proximal stopper tabs 702, which may be configured to be folded or bent inward, as shown, to prevent proximal movement of the sensor device 704 contained within the cage structure 710 beyond the axial position of the tab/stopper-feature(s) 702. The term “suture” is used herein according to its plain and ordinary meaning and may refer to any elongate cord strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord” and “suture” may be used substantially interchangeably. In addition, use of the singular form of any of the suture-related terms listed above, including the terms “suture” and “cord,” may be used to refer to a single suture/cord, or to a portion thereof.

It should be understood that distal (and/or proximal) suture-engagement tabs 705 like those shown in FIGS. 71A and 71B may be incorporated in any of the sensor-retention structures disclosed herein. The suture tabs 705 and/or suture(s) 706 may be configured to hold the sensor 714 in a substantially stationary axial position, with little or no axial movement/wiggle when implanted in the target environment. Such stationary maintenance of the sensor 714 can reduce the effects and/or occurrences of the suture 706 and sensor element 709 coming into forcible contact with one another.

FIG. 72 illustrates a perspective view of a sensor-support strut 728, which may be attached to and/or associated with a medical implant device via an arm structure 721, as discussed in connection with various embodiments of the present disclosure. The sensor-support strut 728 may be configured to support a sensor device 724 in some manner. For example, as shown in FIG. 72, the sensor device 724 may have a cloth or other type of covering 723 disposed around at least a portion thereof. For example, the cloth 723 may substantially envelop the sensor device 724. The covering 723 may wrap around the side portion(s) of the sensor device 724, while allowing an opening therein on a distal and/or proximal side of the sensor device 724 to allow for exposure of a sensor element 729. For example, a distal end of the sensor device 724 may be exposed, as shown in FIG. 72. In some embodiments, a ridge or other features 727 may be associated with the sensor device 724, over which the cloth/covering 723 may be at least partially wrapped or covered. In some embodiments, the sensor-support strut 728 includes a plurality of apertures 726 through which sutures 725, wires, or other elongate material(s)/device(s) may be passed, wherein such sutures 725 or other material(s)/device(s) are sewn and/or passed at least partially through various portions of the cover 723 to thereby secure the sensor device 724 to the sensor-support struts 728. Although a plurality of suture stitches 725 are shown, any number of stitches may be used, including a single stitch.

In some embodiments, a drawstring-type feature may be associated with the distal wrapped portion 727 of the cloth/covering 723, which may allow for cinching/tightening of the cloth/covering 723 around the perimeter of the distal surface of the sensor element 729, thereby constraining axial movement thereof.

### Sensor-Retention Encasements/Housings

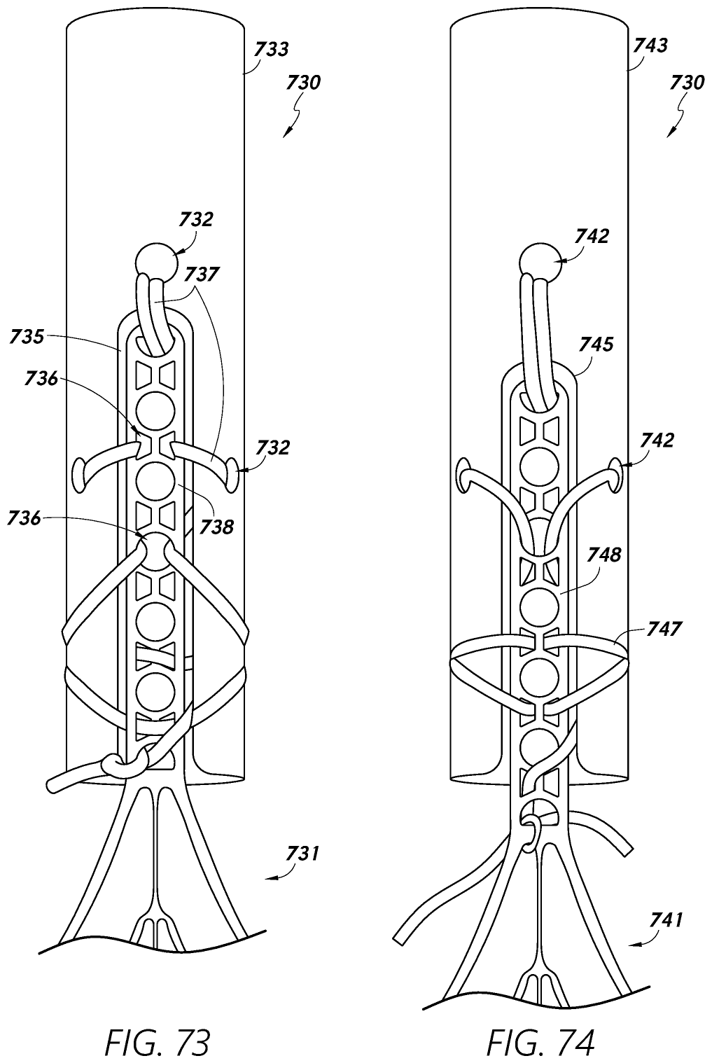

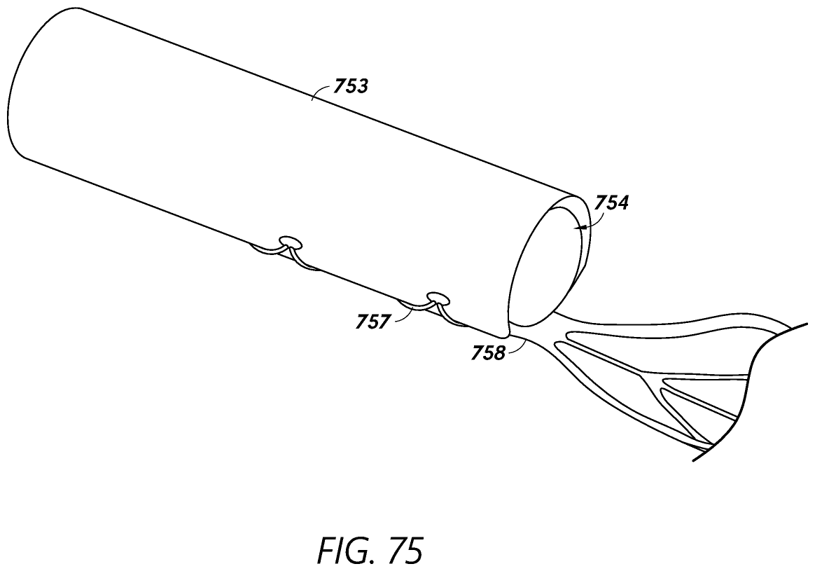

FIGS. 73-75 illustrate perspective views of embodiments of sensor-retention structures 730, 740 including a sensor-support strut 738, 748 and an encasement 733, 743 configured to be secured in some manner to the sensor-support strut 738, 748 and house or enclose at least a portion of a sensor device therein. The encasements 733, 743 can comprise any suitable or desirable material, such as polyether ether ketone (PEEK) or other type of thermoplastic polymer (e.g., other type of polyaryletherketone (PAEK)). With reference to FIG. 73, the encasement 733 can include a cutout 735 configured to fit the sensor-support strut 738, as shown in the illustrated diagram of FIG. 73. The embodiment of FIG. 74 is similar in certain respects to the embodiment shown in FIG. 73 in certain respects. However, in comparison to the encasement 733 FIG. 73, the encasement 743 of FIG. 74 may include a cutout 745 that is shorter in length than the cutout 735 in FIG. 73. Furthermore, the encasement 743 may be secured to the sensor-support arm 741 in a manner such that the encasement 743 is projected, disposed, and/or secured axially farther out relative to the sensor-support strut 738. With respect to both FIGS. 73 and 74, the encasement 733, 743 may include one or more apertures 732, 742, through which sutures, wires, and/or other materials/devices may be sewn/passed, wherein such sutures/materials may further be passed/sewn through apertures/features in the sensor-support strut 738, 748, as shown, to thereby at least partially secure the encasement 733, 743 to the sensor-support strut 738. The encasement 733, 743, 753 may have a sensor device disposed therein at the time of suturing the encasement to the respective sensor-support strut 738, 748, 758.

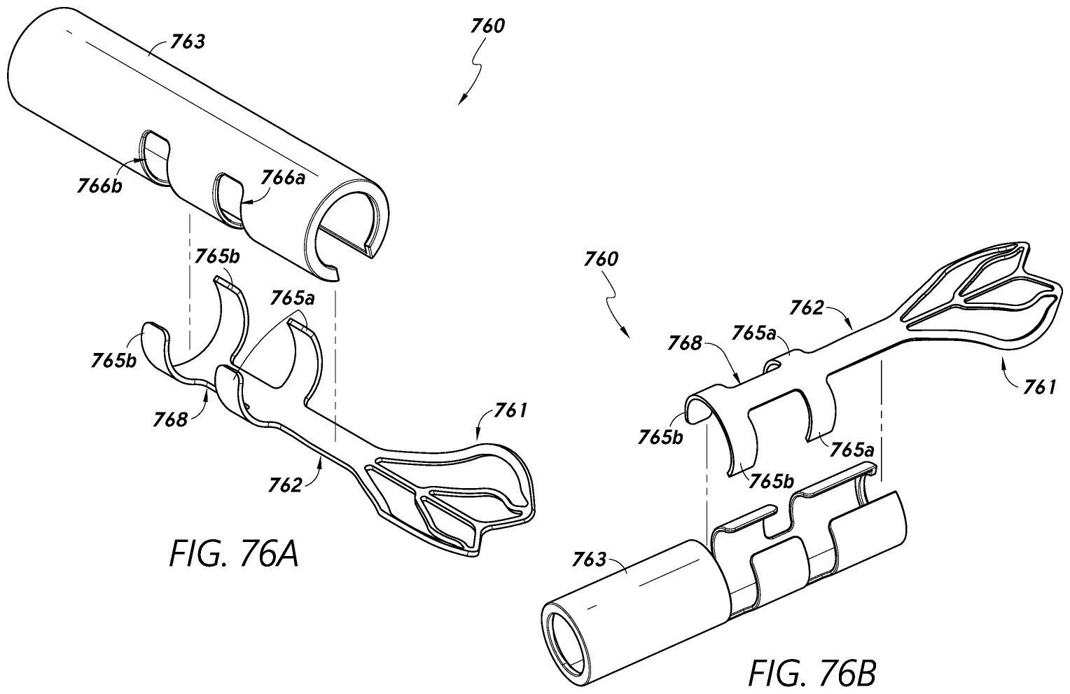

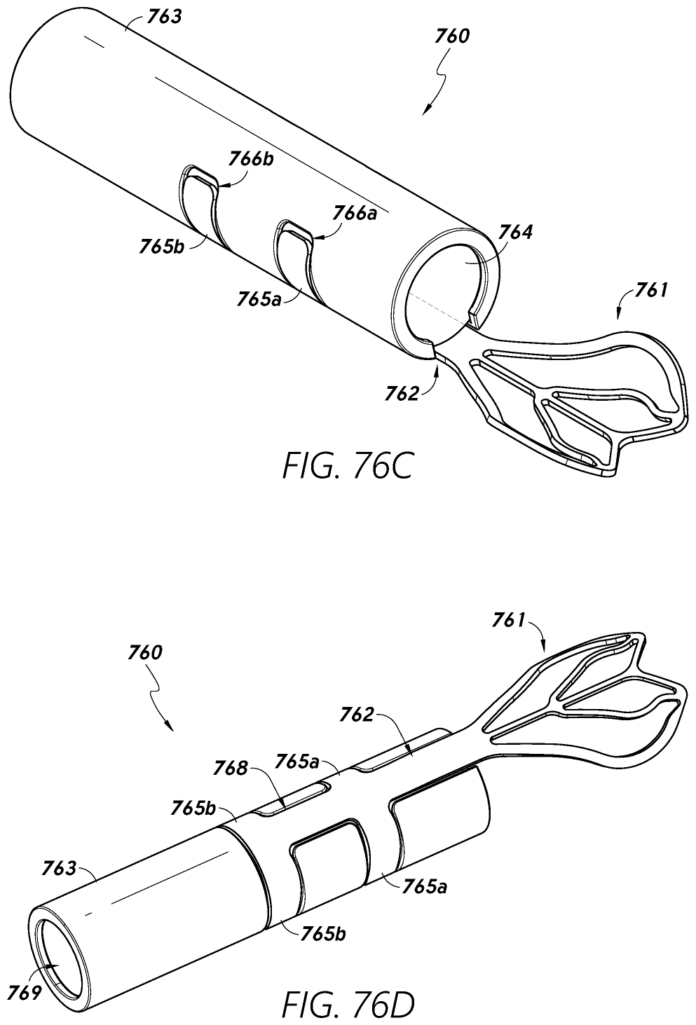

FIGS. 76A through 76D illustrate exploded and perspective views, respectively, of a sensor-retention structure 760 including a sensor-support arm 768 and an encasement 763 configured to fit or engage with the sensor-support arm 768 in some manner and house or enclose at least a portion of a sensor device therein in accordance with one or more embodiments of the present disclosure. The sensor-support arm 768 can include a plurality of sets of opposing fingers 765. For example, as illustrated, the sensor-support arm 768 may include proximal 765a and distal 765b sets of sensor-retention fingers, wherein the encasement 763 includes corresponding cut-outs 766 configured to fit or receive the fingers 765 when the encasement 763 is disposed/placed on the sensor-support arm 768, as shown in FIGS. 76C and 76D. The encasements 763 can comprise any suitable or desirable material, such as polyether ether ketone (PEEK) or other type of thermoplastic polymer (e.g., other type of polyaryletherketone (PAEK)). The cut-outs 766 advantageously can provide axial retention of the encasement 763, and therefore of the sensor 764 disposed therein.

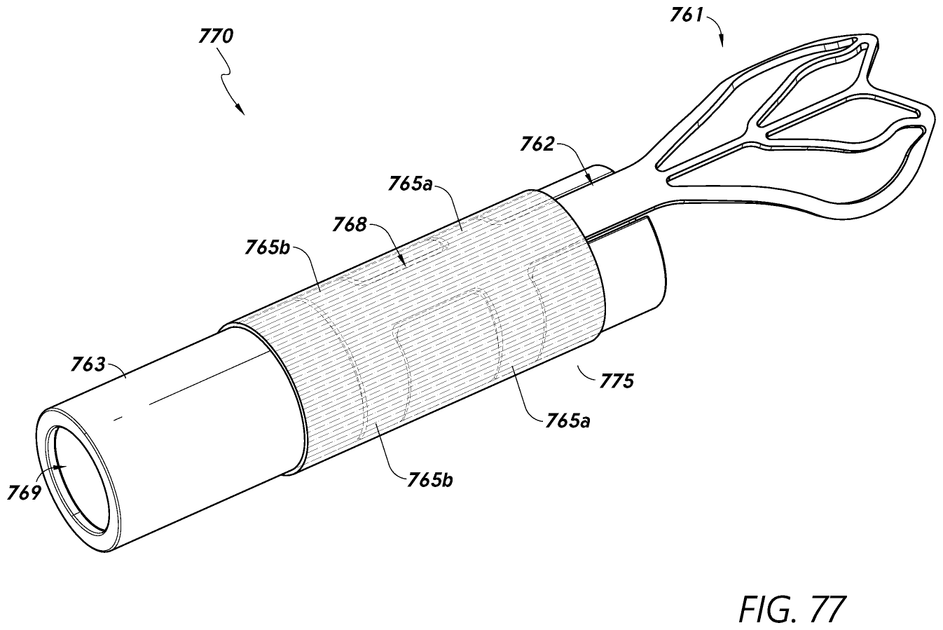

FIG. 77 illustrates a perspective view of the sensor-retention structure 760 of FIGS. 76A through 76D with a sensor device 769 retained therein, wherein a sleeve 775 of any suitable or desirable material, such as thermoplastic polyurethane (TPU), is disposed at least partially around the support arm 768, the finger(s) 765, and/or the encasement 763. The sleeve material 775 may be applied to the encasement 763, finger(s) 765, and/or support arm 768 and may or may not be re-flowed, as described herein, after application thereof. The sleeve 775 can advantageously prevent or constrain the fingers 765 from expanding outwardly, thereby further securing the fingers 765 with the encasement 763 and/or sensor 769.

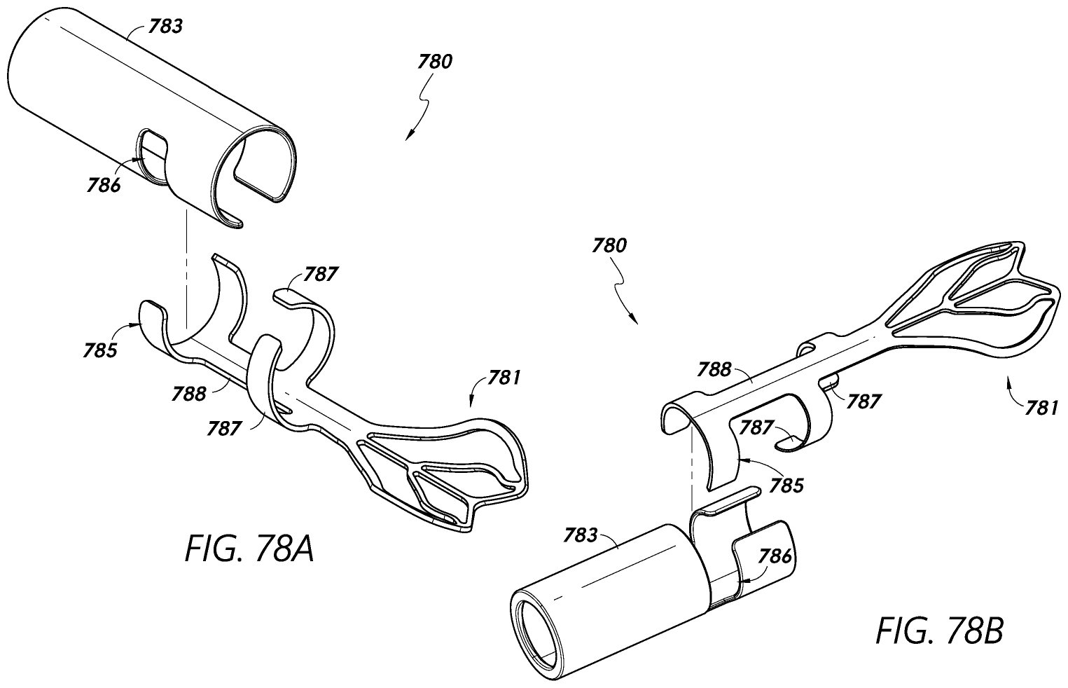

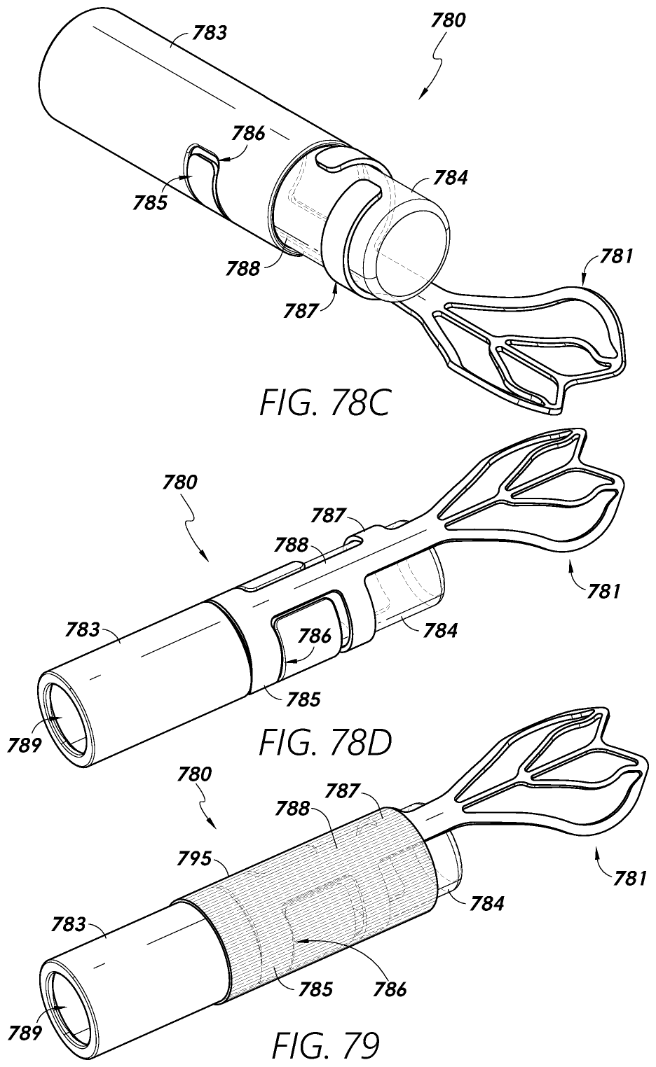

FIGS. 78A through 78D illustrate exploded and perspective views, respectively, of a sensor-retention structures 780 including a sensor-support arm 788 and an encasement 783 configured to fit or engage with the sensor-support arm 788 in some manner and house or enclose at least a portion of a sensor device 784 (see FIGS. 78C and 78D) therein in accordance with one or more embodiments of the present disclosure. The sensor-support arm 788 can include one or more sets of opposing fingers 785 and/or one or more axially-offset retention fingers 787. In some embodiments, the offset fingers 787 are longer than the opposing fingers 785 and are configured to wrap at least partially around/over the opposite half of the cylindrical sensor 784 with respect to the sensor arm 788 when the sensor 784 is retained/disposed therein/thereon as shown in FIGS. 78C and 78D. The encasement 783 can include cutouts 786 configured to fit or receive the fingers 785 when the encasement 783 is disposed/placed on the sensor-support arm 788, as shown in FIGS. 78C and 78D. The encasements 783 can comprise any suitable or desirable material, such as polyether ether ketone (PEEK) or other type of thermoplastic polymer (e.g., other type of polyaryletherketone (PAEK)). The encasement 783 may be shorter than the encasement associated with the embodiments of FIGS. 76A through 76D. Therefore, in some embodiments, the encasement 783 may not cover the entire blanks of the sensor 784. The cut-outs 786 advantageously can provide axial retention of the encasement 783, and therefore of the sensor 784 disposed therein.

FIG. 79 illustrates a perspective view of the sensor-retention structure 780 of FIGS. 78A through 78D with a sensor device 784 retained therein, wherein a sleeve 785 of any suitable or desirable material, such as thermoplastic polyurethane (TPU), is disposed at least partially around the support arm 788, the finger(s) 785, the finger(s) 787, the encasement 783, and/or the sensor device 784. The sleeve material 795 may be applied to the encasement 783, finger(s) 785, 787, and/or support arm 788 and may or may not be re-flowed, as described herein, after application thereof. The sleeve 795 can advantageously prevent or constrain the fingers 785, 787 from expanding outwardly, thereby further securing the fingers 785, 787 with the encasement 783 and/or sensor 784.

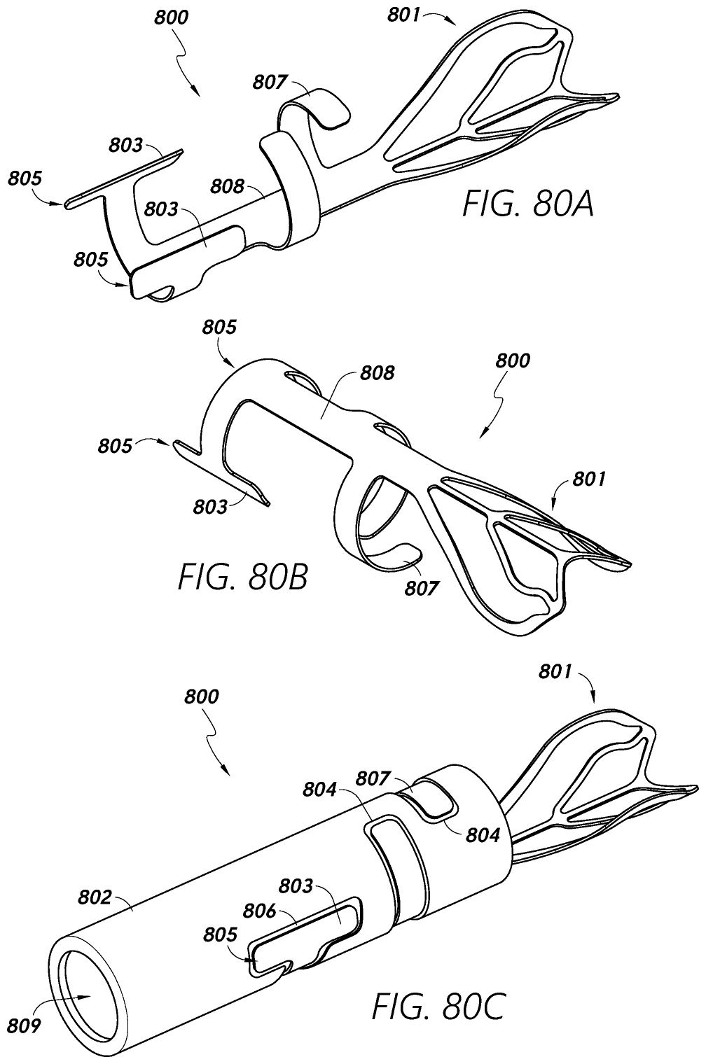

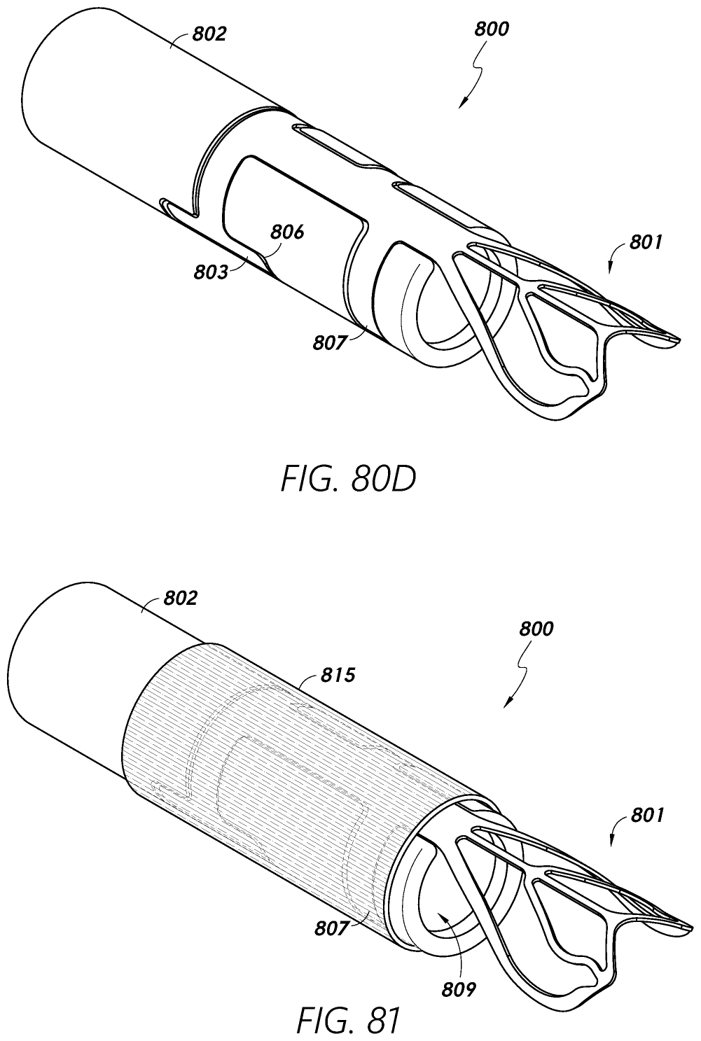

FIGS. 80A through 80D illustrate exploded and perspective views, respectively, of a sensor-retention structures 800 including a sensor-support arm 808 and an encasement 802 configured to fit or engage with the sensor-support arm 808 in some manner and house or enclose at least a portion of a sensor device 809 (see FIGS. 80C and 80D) therein in accordance with one or more embodiments of the present disclosure. The sensor-support arm 808 can include one or more sets of opposing fingers 805 and/or one or more axially-offset retention fingers 807. The fingers 805 can each have associated therewith a respective end crosspiece 803. In some embodiments, the offset retention fingers 807 are longer than the opposing fingers 805 and are configured to wrap at least partially around/over the opposite half of the cylindrical sensor 804 with respect to the sensor arm 808 when the sensor 809 is retained/disposed therein/thereon as shown in FIGS. 80C and 80D. The encasement 802 can include cutouts 806 configured to fit or receive the fingers 805, including the crosspieces 803, when the encasement 802 is disposed/placed on the sensor-support arm 808, as shown in FIGS. 80C and 80D. The encasement 802 can further include cutouts 804 configured to fit or receive the fingers 807 when the encasement 802 is disposed/placed on the sensor-support arm 808. The encasements 802 can comprise any suitable or desirable material, such as polyether ether ketone (PEEK) or other type of thermoplastic polymer (e.g., other type of polyaryletherketone (PAEK)).

FIG. 81 illustrates a perspective view of the sensor-retention structure 800 of FIGS. 80A through 80D with a sensor device 809 retained therein, wherein a sleeve 815 of any suitable or desirable material, such as thermoplastic polyurethane (TPU), is disposed at least partially around the support arm 808, the finger(s) 815, the finger(s) 807, the encasement 802, and/or the sensor device 804. The crosspieces 803 can serve to increase the contact between the fingers 805 and the encasement 802, the sleeve 815 and sensor 809.

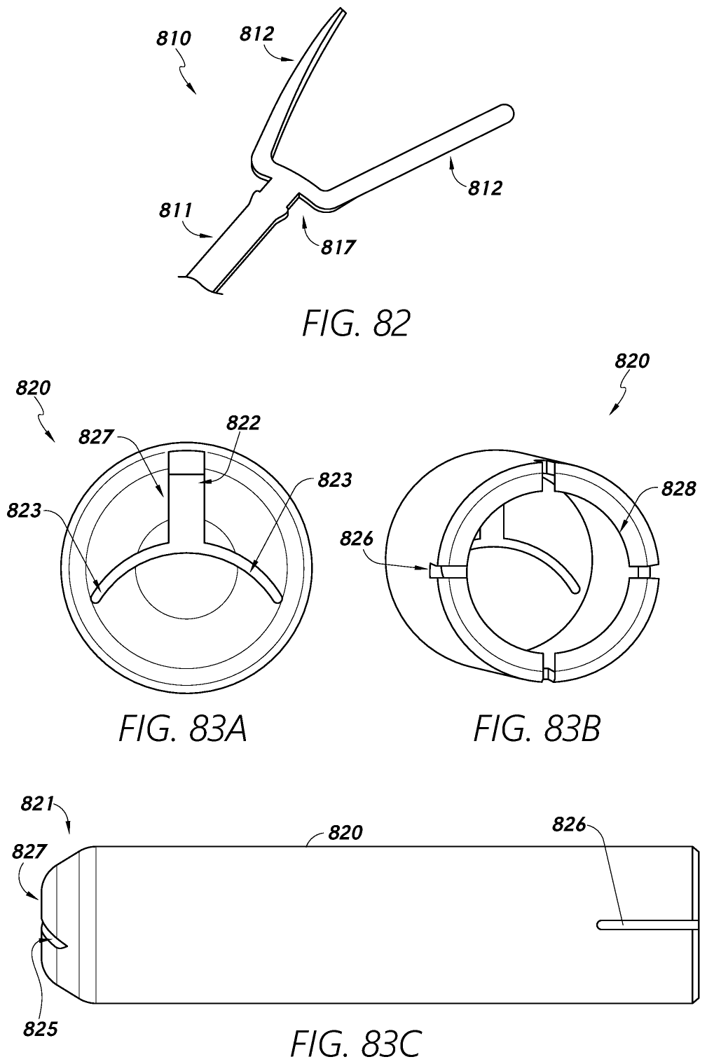

FIG. 82 illustrates a perspective view of a locking retention arm 810 in accordance with one or more embodiments of the present disclosure. The locking retention arm 810 can comprise a plurality of fingers/prongs 812, which may be configured to be locked and/or otherwise secured to and/or in a sensor-retention structure 820, such as an encasement-type structure or the like. FIGS. 83A and 83B illustrate views of a sensor-retention structure 820 that is configured to be secured in some manner to a retention arm structure, such as to the locking sensor-retention arm 810 of FIG. 82. The retention structure 820 advantageously includes certain features 827 configured to engage at least in part and/or in some manner with features of the locking retention arm 810. Such features 827 may advantageously be associated with a proximal end of the retention structure 820 in some embodiments. FIG. 83C shows the retention structure 820 in a side view in accordance with one or more embodiments.

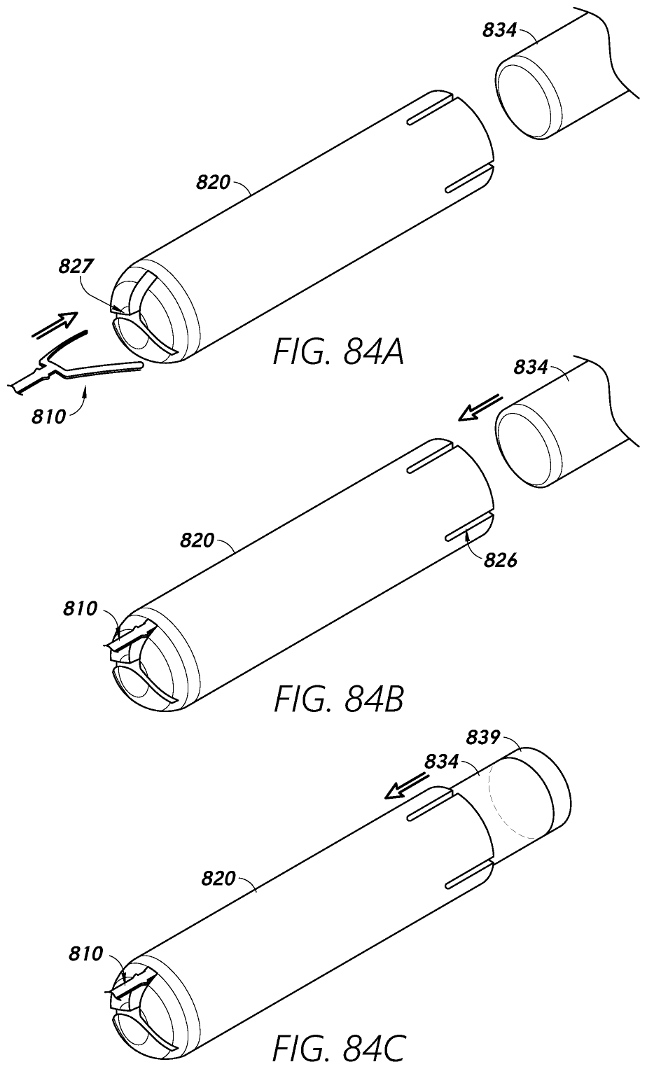

FIGS. 84A through 84C show perspective views of at least a portion of the locking retention arm 810, the retention structure 820, and/or a sensor device 834 that is configured to be disposed at least partially within the retention structure 820 and/or otherwise secured/attached in some manner thereto. The images of FIGS. 84A through 84C respectively corresponds to different stages of a sensor-securing process in accordance with one or more embodiments of the present disclosure.

FIG. 84A shows the locking retention arm 810, the 460 retention structure 820, and the sensor device 834 in a substantially detached or disassociated configuration prior to securing the respective components to one another. As shown in FIG. 84A, the retention structure 820 may have a generally-cylindrical shape or form with dimensions configured to allow for insertion at least partially therein of a generally-cylindrical sensor device 834.

FIG. 84B shows the locking retention arm 810 advanced/inserted into proximal locking features 827 of the retention structure 820 that are configured and/or dimensions to have the prongs 812 of the locking retention arm 810 inserted at least partially therethrough. In some implementations, the locking features 827 of the retention structure 820 include first and second channels 823 dimensioned and configured to receive the prongs 812, wherein the channels 823 join at a common vertical channel 822, such that a trunk feature 817 of the locking retention arm 810 that abuts the prongs 812 can slide therein with the prongs 812 being disposed at least in part within the retention structure 820. In some embodiments, the retention structure 820 may be considered and/or provide a housing or encasement for housing/encasing the sensor device 834, at least in part.

FIG. 84C shows the sensor device 834 being inserted into the retention structure 820. For example, the retention structure 820 may have one or more slots 826 associated with a distal end thereof, wherein such slots(s) allow for diametrical expansion (e.g., outward radial expansion) of the distal end of the retention structure 820 to thereby facilitate insertion of the proximal end of the sensor device 834 into the retention structure 820. In some embodiments, the retention structure 820 includes certain ridge or other lip-type locking/stopping features 828, which may project to some degree radially inward at the distal end/edge of the retention structure 820. Such features 828 may advantageously serve to prevent the sensor device 834 from sliding distally past the stopper features 828.

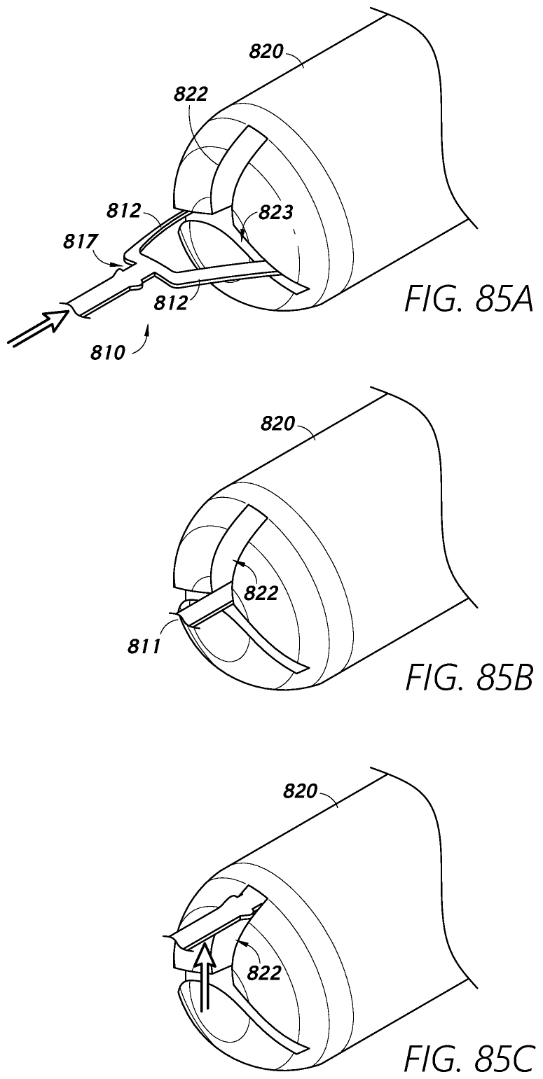

According to some implementations, FIGS. 85A through 85C represent a process for inserting the locking retention arm 810 into a proximal end portion of the retention structure 820 and locking or otherwise securing the arm 810 thereto, as shown. As shown in FIG. 85A, the locking retention arm 810 may be inserted into the proximal receiving channels 823, which may be dimensioned and/or configured to receive the prongs-type extensions of the locking retention arm 810 therein. In FIG. 85B, the single form trunk/base 817 it is advanced into the channels at a point where the side channels 823 and the vertical channel 822 join. As shown in FIG. 85C, the base/trunk 817 of the locking retention arm 810 be drawn upward through the vertical channel 822, thereby causing the prongs 812 to likewise be drawn upward within the retention structure 820. In some implementations, the prongs 812, after sliding the locking retention arm 810 up the vertical channel 822, rest against or near the inside wall of the retention structure 820. In some embodiments, the prongs 812 have certain curvature features that correspond to the curvature and/or surface of the inside of the retention structure 820, such that the prongs 812 may assume a relatively low/small profile against the inside surface of the retention structure 820 when the locking retention arm 810 is engaged with the retention structure 820 as shown in FIG. 85C.

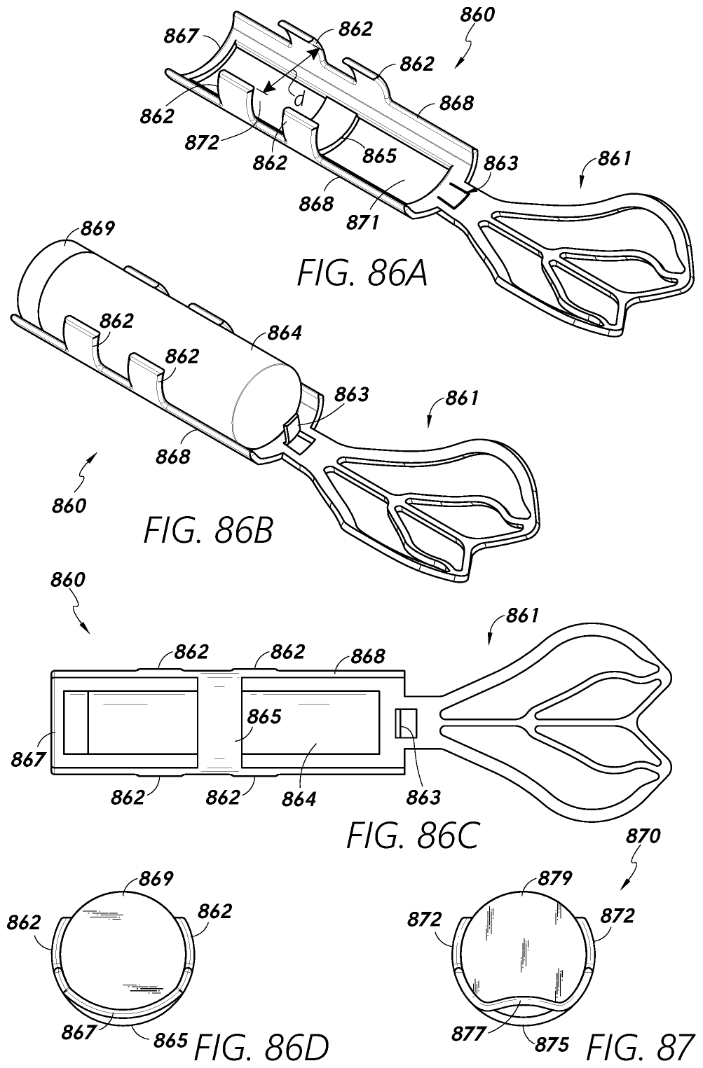

FIGS. 86A-86D illustrate perspective views of a sensor-retention structure 860 configured to hold a sensor device 864 in accordance with one or more embodiments of the present disclosure. The sensor-retention structure 860 may comprise side supports 868 having a certain curvature designed to hold or cradle the cylindrical sensor device 864 therein. For example, the curvature of the side support structures 868 may be similar to or match the curvature of the sensor cylinder 864, such that when the sensor 864 is placed on the support structures 868, the contact area between the sensor-retention structure 860 and the sensor cylinder 864 is distributed over an area of the support structure 860 that faces the sensor cylinder 864. For example, support for holding the sensor device 864 may be provided by the side support structures 868, as well as a medial cross support 865, which may likewise have a certain curvature corresponding to a curvature of the sensor cylinder 864.

The sensor-retention structure 860 may further comprise one or more sensor-retention fingers 862, which may be configured to clamp and/or hold around side portions of the sensor cylinder 864. The sensor-retention fingers 862 may likewise have a certain curvature corresponding to the curvature of the sensor cylinder 864. References herein to curvature of certain structural components of a sensor-retention structure and/or sensor device may be understood to refer to a radius of curvature of such components with respect to an axis, such as the central axis of the sensor cylinder 864 when the cylinder 864 is placed/held in the sensor-retention structure 860. In some embodiments, opposite-facing sensor-retention fingers 862 may be separated at distal ends thereof by a distance d that is less than the diameter of the sensor cylinder 864. In such embodiments, the gap between the opposite-facing sensor-retention fingers 862 may need to be expanded to some degree to allow placement of the sensor cylinder 864 therein. For example, the sensor-retention fingers 862 may be configured to allow the sensor cylinder 864 to be snapped into place between the sensor at retention fingers 862, thereby displacing the sensor at retention fingers 862 radially by some amount when snapping the sensor device 864 into place. The sensor-retention fingers 862 can have shape memory characteristics causing them to return to their non-expanded positions around the sensor cylinder 864 when not presently being displaced thereby. Alternatively, in some implementations, the sensor cylinder 864 may be slid into place within the sensor at retention fingers 862 from a distal or proximal direction.

The sensor-retention structure 860 may further comprise one or more axial stopper features, such as a distal stopper 867 and/or a proximal stop 863. With respect to the distal stopper feature 867, such feature may comprise a crossbar having a radius of curvature that is less than that of the sensor cylinder 864, sensor-retention fingers 862, side support structures 868, and/or medial cross support 865. The distal stopper 867 may lie axially beyond the distal face 869 of the sensor element/membrane of the sensor device 864 when the sensor 864 is placed in the sensor-retention structure 860. Due to the relative flatness of the distal stopper 867 compared to the curvature of the sensor cylinder 864 and/or components of the sensor-retention structure 860, the distal stopper bar 867 may overlap the distal face 869 of the sensor radially by some amount, as shown in FIG. 86D, such that the stopper 867 contacts the distal end/face 869 of the sensor, thereby impeding axial movement/sliding thereof beyond the contact between the stopper bar 867 and the sensor end/face 869. In some embodiments, the distal stopper bar 867 advantageously covers only an outer perimeter portion of the distal end/face 869 of the sensor to thereby reduce the impact of the contact with the stopper bar 867 on the function of the sensor element. For example, in some embodiments, the outer perimeter of the sensor element 869 may comprise, or be associated with, a seal ring component, wherein an inner membrane, such as may operate according to a piezoresistive or capacitive pressure sensor device as a diaphragm thereof, is not covered by the distal stopper bar 867.

With respect to the proximal stopper feature 863, such feature may comprise a tab-type structure or form configured to project radially inward with respect to the curvature of the sensor-retention structure 860, thereby encroaching into the radial space overlapping the proximal surface/face of the sensor 864 and impeding or preventing proximal sliding/movement of the sensor 864 past the proximal stopper tab 863. The stopper tab 863 may be laser-cut or otherwise formed in the form of the sensor-retention structure 860, and may be bent inward/up through shape memory action/movement, or may be manually/mechanically bent or folded into the stopper configuration shown in FIG. 86B using certain tool(s) or through manual manipulation.

FIG. 87 shows an alternative implementation of a distal stopper bar. The distal stopper bar 877 of FIG. 87 may be implemented in connection with any of the embodiments of sensor-retention structures disclosed herein. The distal stopper bar 877 may be formed of a distal circumferential band of a sensor-retention structure (e.g., the sensor-retention structure 860 shown in FIGS. 86A-86D), wherein the stopper bar 877 can be bent at or near a central portion thereof radially inward with respect to an axis of a sensor device 879 disposed in the sensor-retention structure 870 and/or with respect to an axis of curvature of the sensor-retention structure 870, including, for example, certain sensor-retention fingers 872 and/or support bands/forms 875 of the sensor-retention structure 870. Bending the central portion of the stopper band 877 radially inward, as shown in FIG. 87, can cause the stopper bar 877 to radially overlap with the face of the sensor device 879, thereby impeding axial sliding/movement of the sensor device 879. The bending of the distal stopper bar 877 may occur substantially automatically in response to shape-memory characteristics/action of the bar 877, or may be manually bent (e.g., using certain tool(s)) in vivo or prior to introducing the sensor implant device into the body of the patient.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A sensor-retention structure comprising:
a sensor-support arm configured to have disposed thereon an at least partially cylindrical sensor device; and
one or more sensor-retention fingers projecting from the sensor-support arm and configured to be secured to the sensor device.

2. The sensor-retention structure of claim 1, wherein the one or more sensor-retention fingers are configured to be wrapped at least partially around a sensor device disposed on the sensor-support arm.

3. The sensor-retention structure of claim 1, wherein at least one of the one or more sensor-retention fingers comprises a strap form and at least one of the one or more sensor-retention fingers comprises a buckle form that extends from an opposing side of the sensor-support arm such that the strap form can be inserted through a portion of the buckle form.

4. The sensor-retention structure of claim 1, wherein at least one of the one or more sensor-retention fingers has an aperture therein dimensioned to allow for disposition of adhesive therein to secure the at least one of the one or more sensor-retention fingers to the sensor device.

5. The sensor-retention structure of claim 1, wherein the one or more sensor-retention fingers are positioned in one or sets of aligned opposing fingers.

6. The sensor-retention structure of claim 1, wherein the one or more sensor-retention fingers project distally from the sensor-support arm.

7. The sensor-retention structure of claim 6, wherein the one or more sensor-retention fingers include respective distal cross pieces.

8. The sensor-retention structure of claim 1, wherein at least two of the one or more sensor-retention fingers are configured to be locked together at distal ends thereof.

9. The sensor-retention structure of claim 1, and further comprising a distal stopper associated with a distal end portion of the sensor-support arm.

10. The sensor-retention structure of claim 1, and further comprising an encasement form configured to be disposed at least partially over the sensor device when the sensor device is disposed on the sensor-support arm.

11. The sensor-retention structure of claim 10, wherein the encasement form includes one or more cutouts configured to fit at least one of the one or more sensor-retention fingers.

12. The sensor-retention structure of claim 1, wherein the one or more sensor-retention fingers are part of a removable partial ring form.

13. The sensor-retention structure of claim 1, wherein the one or more sensor-retention fingers have associated therewith respective tabs that are configured to be projected radially inward.

14. A sensor-retention structure comprising:
a sensor-support arm configured to have disposed thereon an at least partially cylindrical sensor device; and
a cage structure associated with the sensor-support arm, wherein the cage structure is configured to wrap at least partially around a circumferential surface of the sensor device.

15. The sensor-retention structure of claim 14, wherein the sensor-support arm is attached to a proximal portion of a shunt arm structure, and the sensor-support arm is configured to be bent away from the shunt arm structure to thereby project at least partially radially away from a longitudinal axis of the shunt arm structure.

16. The sensor-retention structure of claim 14, wherein the cage structure includes one or more distal stopper tabs.

17. The sensor-retention structure of any of claim 14, wherein the cage structure comprises a plurality of longitudinal struts.

18. The sensor-retention structure of claim 17, wherein the cage structure comprises a plurality of lateral struts connected between two or more of the plurality of longitudinal struts.

19. The sensor-retention structure of claim 14, and further comprising a sleeve disposed around at least part of the cage structure, wherein the cage structure is in an at least partially wrapped sensor-retention configuration.

20. The sensor-retention structure of claim 14, and further comprising a plurality of suture-attachment tabs associated with a distal end of the cage structure.

21. A sensor-retention structure comprising:
a sensor-support strut; and
a means for securing a sensor device to the sensor-support strut.

22. The sensor-retention structure of claim 21, wherein the means for securing the sensor device to the sensor support strut comprises one or more strap features associated with the sensor-support strut.

23. The sensor-retention structure of claim 21, wherein the means for securing the sensor device to the sensor support strut comprises a cloth wrapped around at least a portion of the sensor device and the sensor-support strut.

24. The sensor-retention structure of claim 21, wherein the means for securing the sensor device to the sensor support strut comprises a polymer film disposed around at least a portion of the sensor device and the sensor-support strut.

25. The sensor-retention structure of claim 21, wherein the means for securing the sensor device to the sensor support strut comprises a sensor mold associated with the sensor-support strut, wherein the sensor mold is configured to have the sensor device inserted therein.

26. The sensor-retention structure of claim 21, wherein the means for securing a sensor device to the sensor-support strut includes a proximal sensor-attachment structure that projects at least partially orthogonally from the sensor-support strut.

27. The sensor-retention structure of claim 26, wherein the proximal sensor-attachment structure includes an aperture therein dimensioned to allow for disposition of adhesive therein to secure the proximal sensor-attachment structure to the sensor device.

28. The sensor-retention structure of claim 26, wherein the proximal sensor-attachment structure comprises a suction cup associated with a distal side thereof.

29. The sensor-retention structure of claim 26, wherein the proximal sensor-attachment structure comprises a distally-angled arm having a hook feature associated with a distal end thereof.

30. The sensor-retention structure of claim 21, wherein the means for securing the sensor device to the sensor support strut comprises:
a housing-attachment flange; and
a sensor housing configured to be attached at a proximal end thereof to the housing-attachment flange and to house the sensor device.

31. The sensor-retention structure of claim 21, wherein the means for securing the sensor device to the sensor support strut comprises one or more trapdoor flaps.

32. The sensor-retention structure of claim 21, wherein the means for securing the sensor device to the sensor support strut comprises a sheet configured to be wrapped around at least a portion of the sensor device.

33. The sensor-retention structure of claim 21, wherein the means for securing the sensor device to the sensor support strut comprises one or more hoop forms configured to be bent away from the sensor-support strut and have sensor device disposed at least partially through openings of the one or more hoop forms.

34. The sensor-retention structure of claim 21, wherein the means for securing the sensor device to the sensor support strut comprises a locking retention arm including a plurality of prongs, and a sensor housing including a plurality of channels associated with a proximal end thereof and configured to receive one or more of the plurality of prongs and a distal end portion of the sensor-support strut.
